(19) United States
(12) Patent Application Publication
(10) Pub. No.: US 2003/0190617 A1
(43) Pub. Date: Oct. 9, 2003

(54) Optineurin nucleic acid molecules and uses thereof

(75) Inventors: Vincent Raymond, Sainte-Foy (CA); Jean Morissette, Sainte-Foy (CA); Erwin Si, Alameda, CA (US)

Correspondence Address: ARNOLD & PORTER, IP DOCKETING DEPARTMENT; RM 1126(b), 555 12TH STREET, N.W., WASHINGTON, DC, 20004-1206, US

(73) Assignee: Erwin Si, Vincent Raymond and Jean Morissette

(21) Appl. No.: 10/091,281

(22) Filed: Mar. 6, 2002

(51) Int. Cl. C12Q 1/68; C12N 5/08; C07H 21/04; C12N 1/21

(52) U.S. Cl. 435/6; 435/325; 435/368; 435/252.3; 536/23.2

(57) ABSTRACT

Promoter sequences of the optineurin gene can be used to diagnose, prognose, and treat glaucoma and related disorders. Methods, kits, and nucleic acids capable of detecting or containing polymorphisms located within the promoter region of the optineurin gene are also provided. The promoter sequences can also be used to generate cells, vectors, and nucleic acids useful in a variety of diagnostic and prognostic methods and kits as well as therapeutic compounds, compositions and methods.

### FIELD OF THE INVENTION

Promoter sequences of the optineurin gene can be used to diagnose, prognose, and treat glaucoma and related disorders. Methods, kits, and nucleic acids capable of detecting or containing polymorphisms located within the promoter region of the optineurin gene are also provided. The promoter sequences can also be used to generate cells, vectors, and nucleic acids useful in a variety of diagnostic and prognostic methods and kits as well as therapeutic compounds, compositions and methods.

### BACKGROUND OF THE INVENTION

The glaucomas are a group of debilitating eye diseases which represent the leading cause of preventable blindness in the United States and other developed nations. Approximately 2.47 million people in the United States and over 67 million people world-wide are estimated to be affected with glaucoma, and over 100,000 Americans are expected to develop this condition every year. Quigley and Vitale, Invest. Ophthalmol. Vis. Sci. 38:83 (1997); Quigley, Br. J. Ophthalmol. 80:389 (1996). Glaucoma is a progressive optic neuropathy characterized by a particular pattern of visual field loss and optic nerve head damage resulting from a number of different disorders that affect the eye. In general, glaucomas are characterized by degeneration of the optic nerve.

Primary Open Angle Glaucoma (POAG), the most common form of glaucoma, is characterized by cupping of the optic nerve head, an altered visual field, and an open iridocorneal angle. Approximately one-half of patients with POAG have high-tension glaucoma, i.e., they exhibit an intraocular pressure (IOP) greater than the normal IOP of about 22 mm Hg. The increased IOP is caused in part by an alteration of the trabecular meshwork (TM), which leads to an obstruction of the normal ability of aqueous humor to leave its chamber surrounding the iris. Elevated IOP can result in progressive visual loss and blindness if not treated appropriately and in a timely fashion.

Because increased IOP is a readily measurable characteristic of glaucoma, the diagnosis of the disease is largely screened for by measuring intraocular pressure (tonometry). Strong, Ophthal. Physiol. Opt. 12:3-7 (1992); Greve et al., Can. J. Ophthamol. 28:201-206 (1993). Unfortunately, because glaucomatous and normal pressure ranges overlap, such methods are of limited value unless multiple readings are obtained. Hitchings, Br. J. Ophthamol. 77:326 (1993); Tuck et al., Ophthal. Physiol. Opt. 13:227-232 (1993); Vaughan et al., In: General Ophthamology, Appleton & Lange, Norwalk, Conn., pp. 213-230 (1992); Vernon, Eye 7:134-137 (1993). Patients may also have a differential sensitivity to optic nerve damage at a given IOP. For these reasons, additional methods, such as direct examination of the optic disk and determination of the extent of a patient's visual field loss are often conducted to improve the accuracy of diagnosis. Greve et al., Can. J. Ophthamol. 28:201-206 (1993). Moreover, these techniques are of limited prognostic value.

Approximately one-third to one-half of patients with POAG consistently have IOP within the statistically normal range of less than 22 mmHg, however. Tielsch et al., JAMA 266:269 (1991); Hitchings, Br. J. Ophthalmol. 76:494 (1992); Grosskreutz and Netland, Int. Ophthalmol. Clin. 34:173 (1994). These patients have been considered to have normal-tension glaucoma (NTG) (also known as low-tension glaucoma (LTG)) and exhibit typical glaucomatous cupping of the optic nerve head and visual field loss. Hitchings and Anderton, Br. J. Ophthalmol. 67:818 (1983). See also Werner, Normal-Tension Glaucoma, in Rich et al., eds. The Glaucomas (2nd ed. 1996): 769-797. NTG has been associated with a disproportionately large amount of cupping, larger than average optic disks, and higher incidences of acquired pit of the optic nerve and optic disk hemorrhage, as compared to high-tension glaucoma patients. Id. at page 774. Because IOP is not elevated in NTG, tonometric techniques are of limited diagnostic and prognostic value, and the disease is often difficult to diagnose until the visual field is significantly impaired.

The present invention relates to a gene known as “optineurin” (for optic neuropathy inducing protein), which is also known variously as: tumor necrosis factor-alpha (TNF-alpha) inducible protein (Li et al., Mol. Cell. Biol. 18:1601 (1998)); FIP-2 (for adenovirus E3-15.7K interacting protein 2); Huntingtin interacting protein L (Faber et al., Hum. Mol. Genet. 7:1463 (1998)), NEMO-related protein (Schwambom et al., J. Biol. Chem. 275:22780 (2000)); transcription factor IRA (TFIIIA) interacting protein (Moreland et al., Nucleic Acids Res. 28:1986 (2000)); and RAB8-interacting protein (Hattula and Peranen, Curr. Bio. 10:1603 (2000)).

Optineurin has been reported as being associated with adult-onset POAG, and mutations in the coding region have been reported as correlated with adult-onset NTG/POAG and an increased risk of glaucoma. Rezaie et al., “Adult-Onset Primary Open Angle Glaucoma Caused by Mutations in OPTN”, Science 295:1077-1079 (2002). Direct interaction of optineurin with E3-14.7K protein has been reported and it has also been reported that such interaction utilizes TNF-alpha or FAS-Ligand pathways to mediate apoptosis, inflammation or vasoconstriction. Li et al., Mol. Cell. Biol. 18:1601 (1998); Wold, J. Cell. Biochem. 53:329 (1993). Optineurin also is reported to function through interactions with other proteins in cellular morphogenesis and membrane trafficking (RAB 8), vesicle trafficking (Huntingtin), transcription activation (TFIIIA), and assembly or activation of two kinases. Li et al., Mol. Cell. Biol. 18:1601 (1998); Hattula and Peranen, Curr. Bio. 10:1603 (2000); Moritz et al., Mol. Biol. Cell 12:2341 (2001); Moreland et al., Nucleic Acids Res. 28:1986 (2000); Schwamborn et al., J. Biol. Chem. 275:22780 (2000).

### SUMMARY OF THE INVENTION

The present invention includes and provides an isolated nucleic acid molecule that comprises at least 20 consecutive nucleotides but not more than 1500 consecutive nucleotides of the sequence of SEQ ID NO: 1. The present invention also includes and provides an isolated nucleic acid molecule comprising a promoter which comprises at least 20 consecutive nucleotides but not more than 1500 consecutive nucleotides of the sequence of SEQ ID NO: 1, the promoter being operably linked to a heterologous nucleic acid sequence. Such heterologous nucleic acid sequences may include, without limitation, coding sequences, toxins, and reporter genes, and also may be capable of being transcribed as an antisense RNA.

The present invention includes a nucleic acid molecule capable of detecting a single nucleotide polymorphism selected from table 1 and a nucleic acid molecule capable of detecting a single nucleotide polymorphism in an optineurin promoter by specifically detecting said single nucleotide polymorphism in the optineurin promoter, where the nucleic acid molecule does not specifically hybridize to a nucleic acid molecule consisting of SEQ ID NO: 1.

Host cells comprising such nucleic acid molecules are also provided by the present invention, including, without limitation, host cells selected from the group consisting of non-human mammalian cells, bacterial cells, and isolated human cells.

The present invention also provides and includes methods for diagnosing glaucoma in a sample obtained from a cell or a bodily fluid by detecting a polymorphism in a promoter region of the optineurin gene, comprising the steps of: (A) incubating under conditions permitting nucleic acid hybridization, a marker nucleic acid molecule, the marker nucleic acid molecule having a nucleic acid sequence that specifically hybridizes to a sequence selected from the group consisting of SEQ ID NO: 1 and a complement thereof, and a complementary nucleic acid molecule obtained from a sample, wherein nucleic acid hybridization between the marker nucleic acid molecule and the complementary nucleic acid molecule permits the detection of said polymorphism; (B) permitting hybridization between the marker nucleic acid molecule and the complementary nucleic acid molecule; and (C) detecting the presence of the polymorphism, wherein the detection of the polymorphism is diagnostic of glaucoma.

Also provided by the present invention are methods for prognosing glaucoma in a sample obtained from a cell or a bodily fluid by detecting a polymorphism in a promoter region of the optineurin gene, comprising the steps of: (A) incubating under conditions permitting nucleic acid hybridization, a marker nucleic acid molecule, the marker nucleic acid molecule having a nucleic acid sequence that specifically hybridizes to a sequence selected from the group consisting of SEQ ID NO: 1 and complement thereof, and a complementary nucleic acid molecule obtained from a sample, where nucleic acid hybridization between the marker nucleic acid molecule and the complementary nucleic acid molecule permits the detection of the polymorphism; (B) permitting hybridization between the marker nucleic acid molecule and the complementary nucleic acid molecule; and (C) detecting the presence of the polymorphism, where the detection of the polymorphism is prognostic of glaucoma.

Further provided by the present invention are methods for diagnosing or prognosing glaucoma in a sample obtained from a cell or a bodily fluid by detecting a polymorphism in a promoter region of the optineurin gene, comprising the steps of: (A) incubating under conditions permitting nucleic acid hybridization, a marker nucleic acid molecule, the marker nucleic acid molecule having a nucleic acid sequence that specifically hybridizes to a optineurin promoter sequence or its complement, and a complementary nucleic acid molecule obtained from a sample, where nucleic acid hybridization between the marker nucleic acid molecule and the complementary nucleic acid molecule permits the detection of the polymorphism; (B) permitting hybridization between the marker nucleic acid molecule and the complementary nucleic acid molecule; and (C) detecting the presence of the polymorphism, where the detection of the polymorphism is diagnostic or prognostic of glaucoma.

The methods of the present invention may be used to detect a single nucleotide polymorphism, and may further comprise a second marker nucleic acid molecule.

The present invention further provides methods for detecting the presence or absence of a SNP sequence variation in a sample containing DNA, comprising contacting a labeled nucleic acid capable of detecting a single nucleotide polymorphism selected from table 1 with the DNA of the sample under hybridization conditions and determining the presence of hybrid nucleic acid molecules comprising the labeled nucleic acid.

The present invention additionally includes and provides methods for detecting the presence or absence of an optineurin promoter sequence variation, for determining the presence of increased susceptibility to a glaucoma, or to a progressive ocular hypertensive disorder resulting in loss of visual field in a patient, or the severity or progression of glaucoma in a patient, and methods for detecting a polymorphism comprising: obtaining a sample containing human genomic DNA, by providing a nucleic acid molecule capable of detecting a single nucleotide polymorphism located with an optineurin promoter, and detecting the presence or absence of said polymorphism.

Further, the present invention provides kits containing agents of the present invention or kits capable of carrying out a method of the present invention including, without limitation, kits for determining the presence of increased susceptibility to a glaucoma, or to a progressive ocular hypertensive disorder resulting in loss of visual field, or the severity or progression of glaucoma in a patient, comprising a labeled nucleic acid capable of detecting a single nucleotide polymorphism selected from table 1 and a means for detecting hybridization with the labeled nucleic acid, and instructions for using a kit and kits for determining the presence of increased susceptibility to a glaucoma, or to a progressive ocular hypertensive disorder resulting in loss of visual field in a patient, or the severity or progression of glaucoma in a patient, comprising amplification reaction primers that direct amplification of a selected nucleic acid region containing the characteristic nucleotide substitution of an optineurin promoter SNP sequence variant and an enzyme for amplifying the region containing the characteristic nucleotide substitution.

### BRIEF DESCRIPTION OF THE DRAWINGS

FIG. 1 depicts the genomic structure of optineurin, including regions which interact with other known proteins, putative functional domains, sizes of exons, and position and types of mutations observed.

FIG. 2 depicts an interaction of optineurin with other proteins and its potential involvement in alternative pathways of FAS-Ligand (left) and TNF-alpha (right). Interactions are depicted with solid arrows; downstream effects are depicted with open arrows; and a blocking effect of one protein on another is depicted with arrows ending in a circle.

FIG. 3 provides a diagrammatic representation of the location of single nucleotide polymorphisms (depicted as an “n” above the polymorphic nucleotide) and DNA motifs (cis elements) and putative regulatory regions (depicted by labeled lines beneath the nucleotides of the motif or regulatory region) and repeat elements (depicted by dotted lines above the nucleotides of the repeat element) in the optineurin promoter sequence (SEQ ID NO: 1).

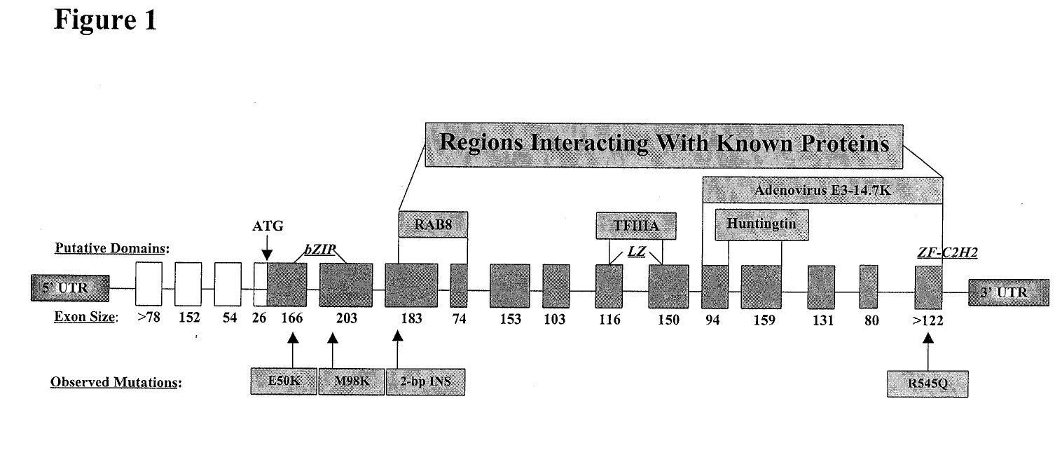

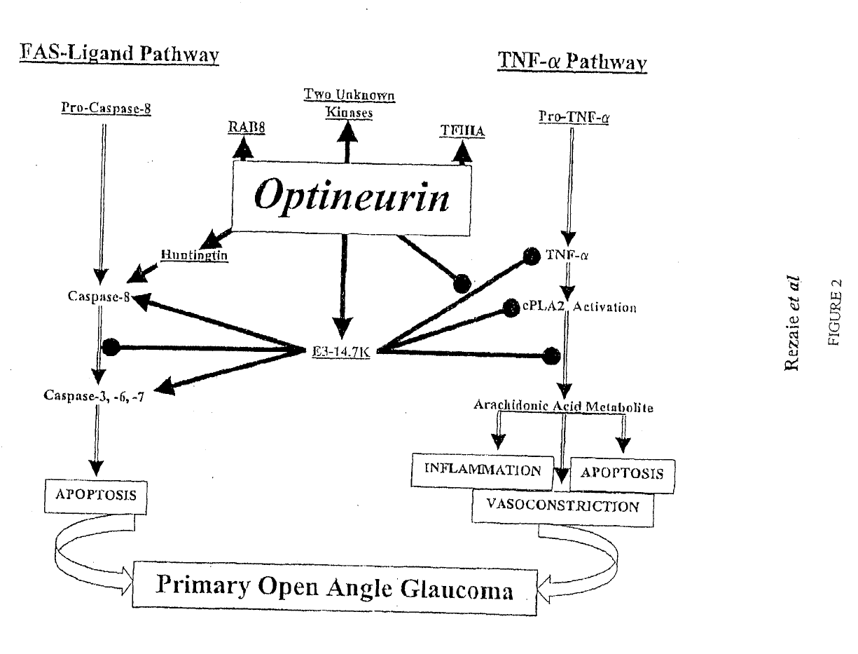

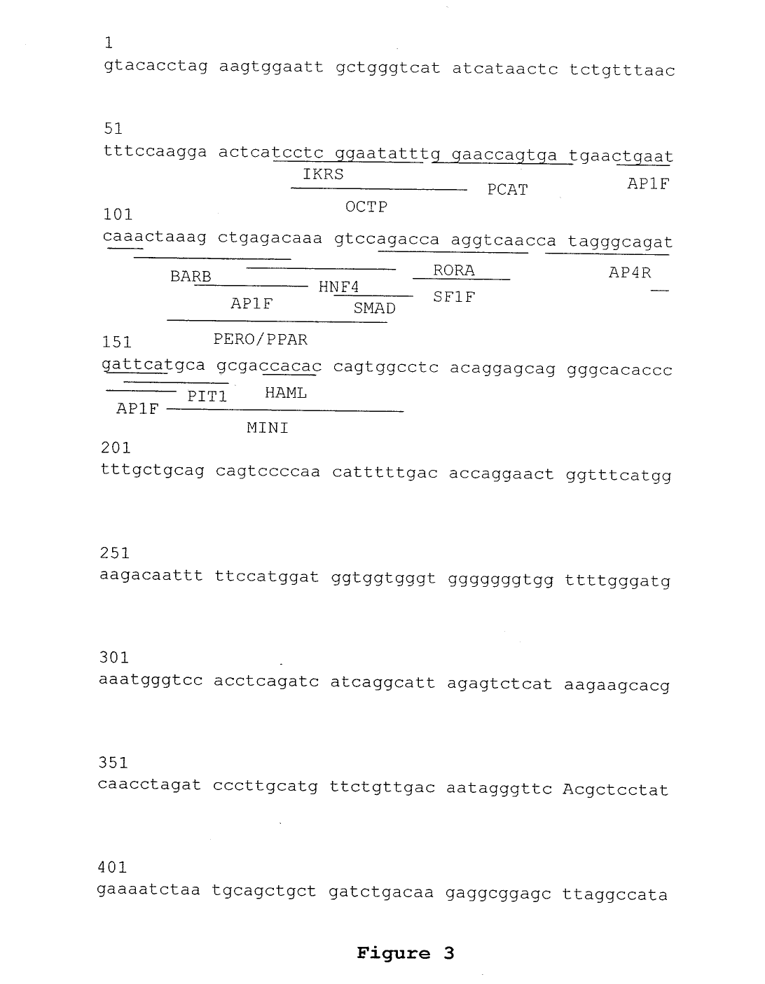

### DESCRIPTION OF THE NUCLEIC AND AMINO ACID SEQUENCES

SEQ ID NO: 1 is a Homo sapiens nucleotide sequence of optineurin promoter.

SEQ ID NO: 2 is a Homo sapiens nucleotide sequence of the optineurin promoter and the optineurin coding region.

SEQ ID NOs: 3 through 463 are Homo sapiens nucleotide sequences of DNA motifs, repeat elements, and putative regulatory regions identified in the human optineurin promoter.

### DEFINITIONS

The following definitions are provided as an aid to understanding the detailed description of the present invention.

The abbreviation “EP” refers to patent applications and patents published by the European Patent Office, and the term “WO” refers to patent applications published by the World Intellectual Property Organization. “PNAS” refers to Proc. Natl. Acad. Sci. (U.S.A.).

“Amino acid” and “amino acids” refer to all naturally occurring L-amino acids. This definition is meant to include norleucine, norvaline, ornithine, homocysteine, and homoserine.

“Chromosome walking” means a process of extending a genetic map by successive hybridization steps.

The phrases “coding sequence,” “structural sequence,” and “structural nucleic acid sequence” refer to a physical structure comprising an orderly arrangement of nucleic acids. The coding sequence, structural sequence, and structural nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

A nucleic acid molecule is said to be the “complement” of another nucleic acid molecule if they exhibit complete complementarity, i.e., every nucleotide of one of the molecules is complementary to a nucleotide of the other. Two molecules are “minimally complementary” if they can hybridize to one another with sufficient stability to remain annealed to one another under at least conventional “low-stringency” conditions. Similarly, the molecules are “complementary” if they can hybridize to one another with sufficient stability to remain annealed to one another under conventional “high-stringency” conditions. Conventional stringency conditions are described by Sambrook et al., Molecular Cloning: A Laboratory Manual, Second Edition, Cold Spring Harbor Laboratory Press, Cold Spring Harbor, N.Y. (1989); Haymes et al., Nucleic Acid Hybridization, A Practical Approach, IRL Press, Washington, D.C. (1985).

The phrases “DNA sequence,” “nucleic acid sequence,” and “nucleic acid molecule” refer to a physical structure comprising an orderly arrangement of nucleic acids. The DNA sequence or nucleic acid sequence may be contained within a larger nucleic acid molecule, vector, or the like. In addition, the orderly arrangement of nucleic acids in these sequences may be depicted in the form of a sequence listing, figure, table, electronic medium, or the like. “Nucleic acid” refers to deoxyribonucleic acid (DNA) and ribonucleic acid (RNA).

“Exogenous genetic material” is any genetic material, whether naturally occurring or otherwise, from any source that is capable of being inserted into any organism.

The term “expression” refers to the transcription of a gene to produce the corresponding mRNA and translation of this mRNA to produce the corresponding gene product (i.e., a peptide, polypeptide, or protein). The term “expression of antisense RNA” refers to the transcription of a DNA to produce a first RNA molecule capable of hybridizing to a second RNA molecule.

As used herein, the term “glaucoma” has its art recognized meaning, and includes primary glaucomas, secondary glaucomas, juvenile glaucomas, congenital glaucomas, and familial glaucomas, including, without limitation, pigmentary glaucoma, high tension glaucoma, low tension glaucoma, normal tension glaucoma, and their related diseases. A disease or condition is said to be related to glaucoma if it possesses or exhibits a symptom of glaucoma, for example, and increased intraocular pressure resulting from aqueous outflow resistance.

“Homology” refers to the level of similarity between two or more nucleic acid or amino acid sequences in terms of percent of positional identity (i.e., sequence similarity or identity).

As used herein, a “homolog protein” molecule or fragment thereof is a counterpart protein molecule or fragment thereof in a second species (e.g., human optineurin is a homolog of mouse optineurin). A homolog can also be generated by molecular evolution or DNA shuffling techniques, so that the molecule retains at least one functional or structure characteristic of the original protein (see, e.g., U.S. Pat. No. 5,811,238).

The phrase “heterologous” refers to the relationship between two or more nucleic acid or protein sequences that are derived from different sources. For example, a promoter is heterologous with respect to a coding sequence if such a combination is not normally found in nature. In addition, a particular sequence may be “heterologous” with respect to a cell or organism into which it is inserted (i.e. does not naturally occur in that particular cell or organism).

“Hybridization” refers to the ability of a strand of nucleic acid to join with a complementary strand via base pairing. Hybridization occurs when complementary nucleic acid sequences in the two nucleic acid strands contact one another under appropriate conditions.

“Isolated” refers to a molecule separated from substantially all other molecules normally associated with it in its native state. More preferably an isolated molecule is the predominant species present in a preparation. A isolated molecule may be greater than 60% free, preferably 75% free, more preferably 90% free, and most preferably 95% free from the other molecules (exclusive of solvent) present in the natural mixture. The term “isolated” is not intended to encompass molecules present in their native state.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence.

“Polyadenylation signal” or “polyA signal” refers to a nucleic acid sequence located 3′ to a coding region that promotes the addition of adenylate nucleotides to the 3′ end of the mRNA transcribed from the coding region.

The term “promoter” or “promoter region” refers to a nucleic acid sequence, usually found upstream (5′) to a coding sequence, that is capable of directing transcription of a nucleic acid sequence into mRNA. The promoter or promoter region typically provide a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed.

The term “protein” “polypeptide” or “peptide molecule” includes any molecule that comprises five or more amino acids. Typically, peptide molecules are shorter than 50 amino acids. It is well known in the art that proteins may undergo modification, including post-translational modifications, such as, but not limited to, disulfide bond formation, glycosylation, phosphorylation, or oligomerization. Thus, as used herein, the term “protein”, “polypeptide” or “peptide molecule” includes any protein that is modified by any biological or non-biological process.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein.

“Recombinant vector” refers to any agent such as a plasmid, cosmid, virus, autonomously replicating sequence, phage, or linear single-stranded, circular single-stranded, linear double-stranded, or circular double-stranded DNA or RNA nucleotide sequence. The recombinant vector may be derived from any source and is capable of genomic integration or autonomous replication.

“Regulatory sequence” refers to a nucleotide sequence located upstream (5′), within, or downstream (3′) to a coding sequence. Transcription and expression of the coding sequence is typically impacted by the presence or absence of the regulatory sequence.

An antibody or peptide is said to “specifically bind” to a protein, polypeptide, or peptide molecule of the invention if such binding is not competitively inhibited by the presence of non-related molecules.

“Substantially homologous” refers to two sequences which are at least 90% identical in sequence, as measured by the BestFit program described herein (Version 10; Genetics Computer Group, Inc., University of Wisconsin Biotechnology Center, Madison, Wis.), using default parameters.

“Transcription” refers to the process of producing an RNA copy from a DNA template.

“Transfection” refers to the introduction of exogenous DNA into a recipient host.

“Transformation” refers a process by which the genetic material carried by a recipient host is altered by stable incorporation of exogenous DNA. The term “host” refers to cells or organisms.

“Transgenic” refers to organisms into which exogenous nucleic acid sequences are integrated.

### DETAILED DESCRIPTION OF THE PREFERRED EMBODIMENTS

One skilled in the art may refer to general reference texts for detailed descriptions of known techniques discussed herein or equivalent techniques. These texts include Ausubel et al., Current Protocols in Molecular Biology, John Wiley and Sons, Inc. (1995); Sambrook et al., Molecular Cloning, A Laboratory Manual (2d ed.), Cold Spring Harbor Press, Cold Spring Harbor, N.Y. (1989); Birren et al., Genome Analysis: A Laboratory Manual, volumes 1 through 4, Cold Spring Harbor Press, Cold Spring Harbor, N.Y. (1997-1999); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990); and Albert and Jakobiec, Principles and Practice of Ophthalmology, W. B. Saunders Company (1994). These texts can, of course, also be referred to in making or using an aspect of the invention.

A. Human optineurin

In the present invention, a human optineurin promoter has been identified. The transcription start site of the optineurin coding sequence was determined, and a 5 kb fragment of genomic sequence upstream of it was cloned. This fragment was found to contain a promoter responsible for the transcription of optineurin (SEQ ID NO: 1).

The present invention provides a number of agents, for example, nucleic acid molecules comprising the optineurin promoter, and nucleic acid molecules comprising key regulatory regions of the optineurin promoter, and provides uses of such agents. The agents of the invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic and thus involve the capacity of the agent to mediate a chemical reaction or response. The agents will preferably be isolated. The agents of the invention may also be recombinant.

It is understood that any of the agents of the invention can be isolated and/or be biologically active and/or recombinant. It is also understood that the agents of the invention may be labeled with reagents that facilitate detection of the agent, e.g., fluorescent labels, chemical labels, modified bases, and the like. The agents may be used as diagnostic or therapeutic compositions useful in the detection, prevention, and treatment of glaucoma.

In one aspect, the invention relates to nucleic acids comprising non-coding regions or promoter regions associated with the optineurin gene of mammals. These nucleic acids can be used in identifying polymorphisms in the genomes of mammals and humans that predict a susceptibility to glaucomas or diseases related to alterations in IOP. A number of diagnostic or prognostic methods and kits can be designed from these nucleic acids including, without limitation those set forth herein.

In one embodiment, the nucleic acids can be used to identify or detect a single base polymorphism in a genome. In other embodiments, two or more single base polymorphisms or multiple base polymorphisms can be identified or detected. The detection of a known polymorphism can be the basis for diagnostic and prognostic methods and kits of the invention. Various methods of detecting nucleic acids can be used in these methods and with the kits, including, but not limited to, solution hybridization, hybridization to microarrays containing immobilized nucleic acids or other immobilized nucleic acids, amplification-based methods such as PCR and the like, and an appropriate biosensor apparatus comprising a nucleic acid or nucleic acid binding reagent.

In another aspect, the invention relates to specific sequences and variants or mutants from the promoter or 5′ regulatory region of the human optineurin gene and nucleic acids incorporating these sequences, variants or mutants. The nucleic acids can be incorporated into the methods and kits of the invention, or used in expression systems, vectors, and cells to produce a protein or polypeptide of interest, or used in methods to identify or detect regulatory proteins or proteins that specifically bind to promoter or regulatory regions of the optineurin gene.

In one embodiment of this aspect of the invention, for example, nucleic acids have an optineurin promoter SNP sequence variant, represented by characteristic nucleotides, as shown in Table 1 below. A nucleic acid incorporating such a characteristic nucleotide can be used to identify and determine individuals at risk for developing glaucoma or a progression from an ocular hypertensive state, and may be associated with therapeutic responsiveness. For example, a SNP in the MYOC gene promter has been reported to modify therapeutic response and be correlated with resistance to treatment. Colomb et al., Clin. Genet. 60:220-225 (2001). The identification of changes in IOP can be done by any known means, however, the “Armaly” criteria is preferred (see Armaly, Arch. Ophthalinol. 70:492 (1963); Armaly, Arch. Ophthalmol. 75:32-35 (1966); Kitazawa et al., Arch. Ophthalmol. 99:819-823 (1981); Lewis et al., Amer. J. Ophthalmol. 106:607-612 (1988); Becker et al., Amer. J. Ophthalmol. 57:543 (1967)).

TABLE 1
Single Nucleotide Polymorphisms (SNPs) in the Optineurin Promoter

| Location in SEQ ID NO:1 | Characteristic Nucleotides |
|---|---|
| 391 | a/g |
| 691 | a/g |
| 709 | a/g |
| 887 | t/a |
| 894 | a/t |
| 987 | a/c |
| 1112 | t/c |
| 1505 | c/cc |
| 1606 | g/a |
| 2405 | g/t |
| 2606 | a/g |
| 3313 | g/a |
| 3555 | t/tt |
| 3625 | a/g |
| 3629 | c/t |
| 3882 | t/tt |
| 3988 | c/t |
| 4452 | g/a |

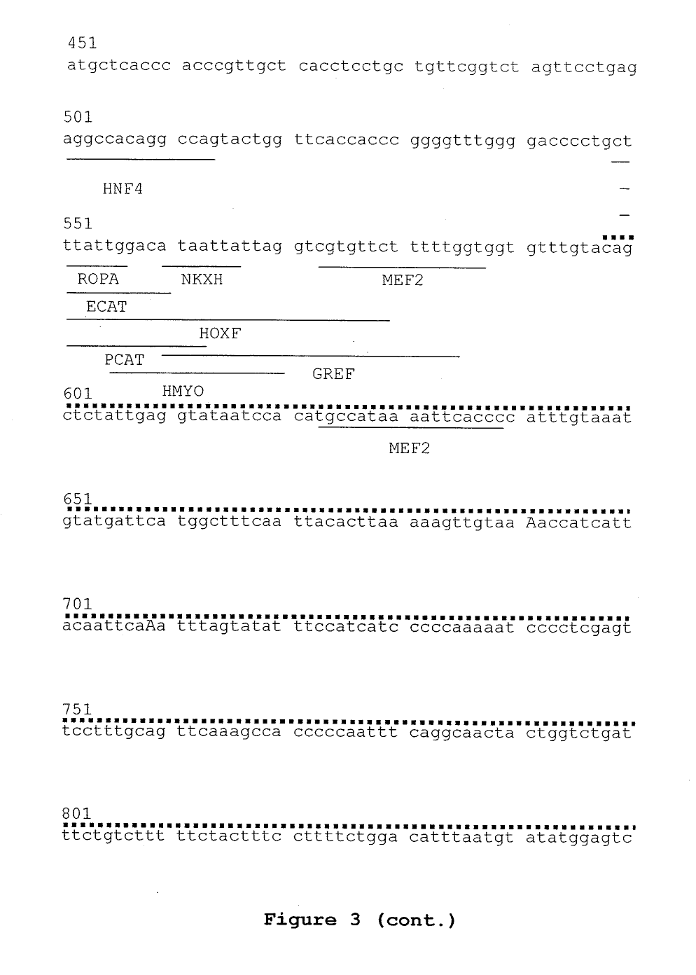

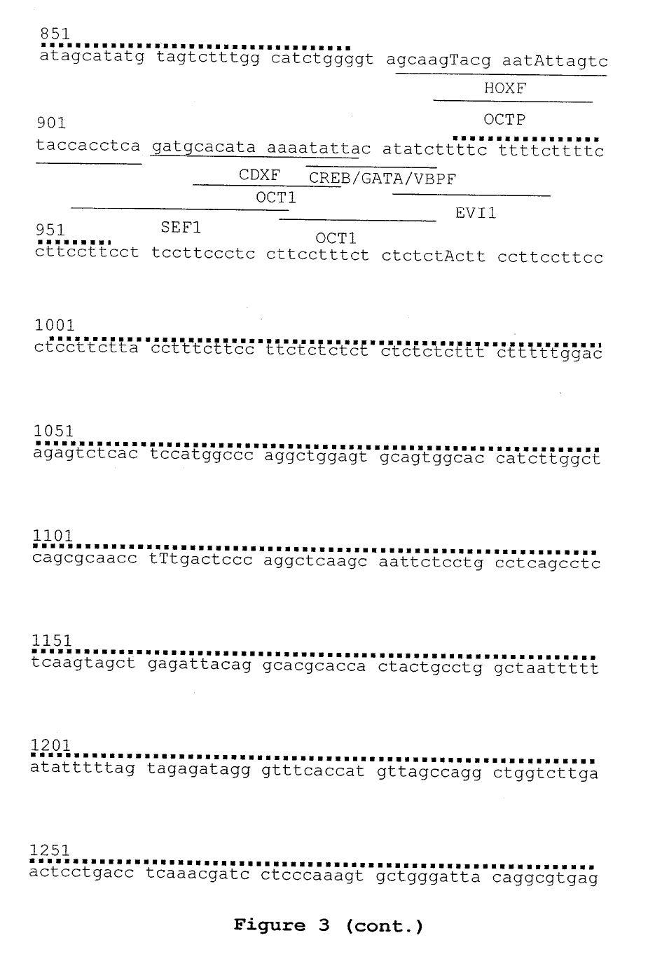

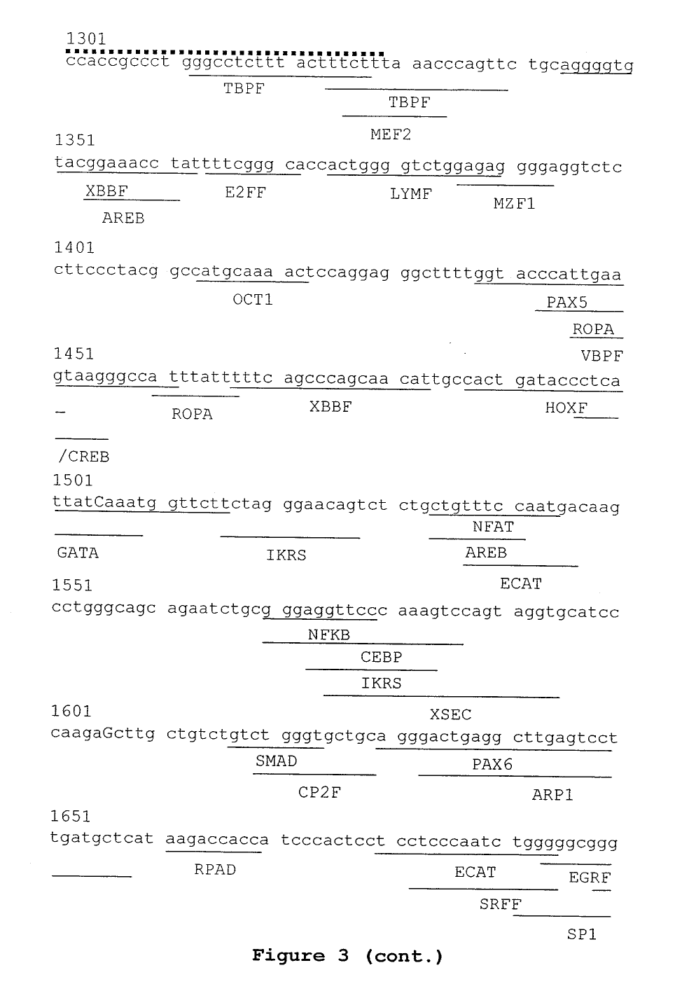

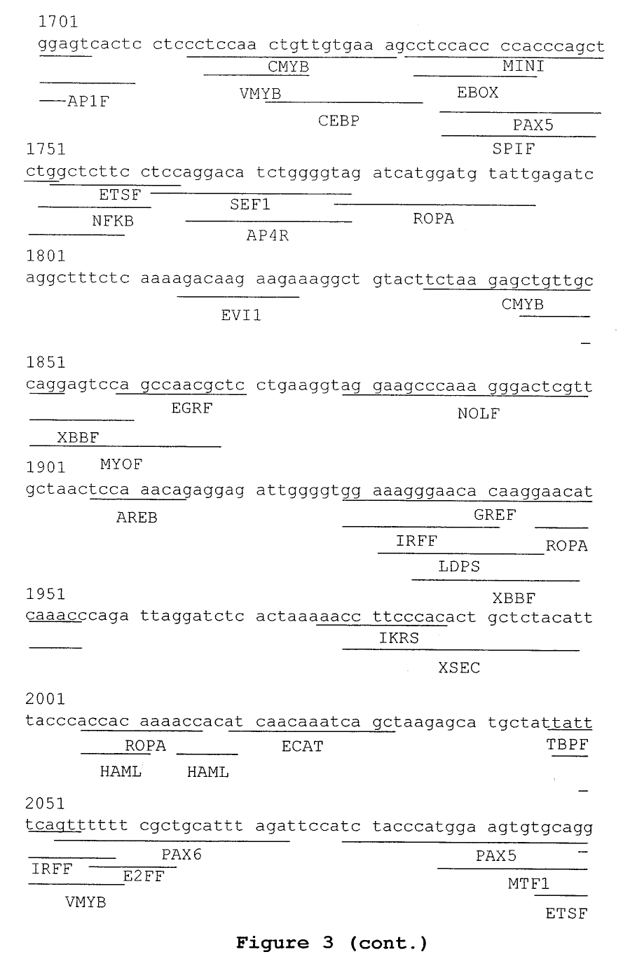

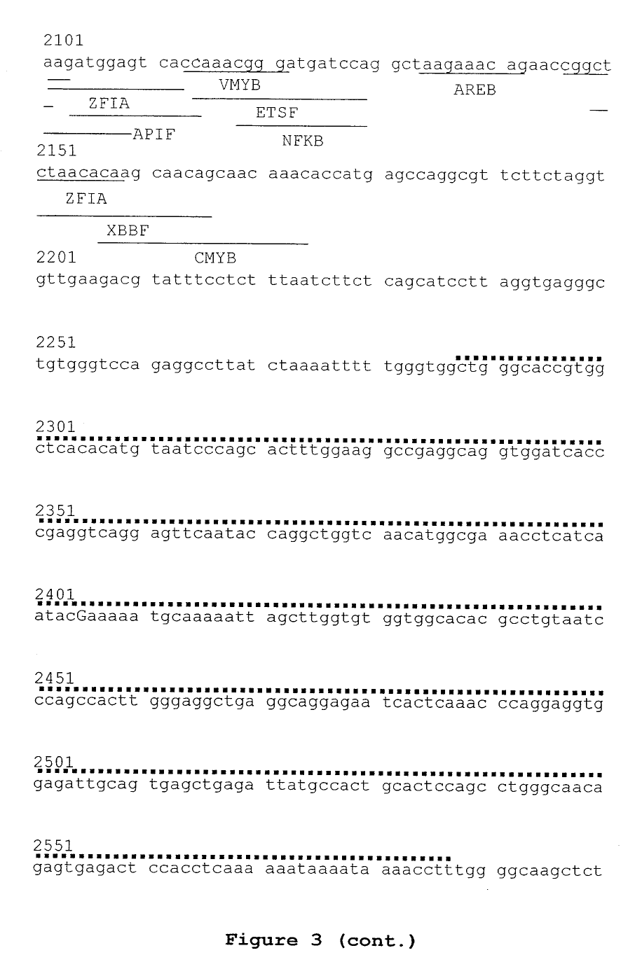

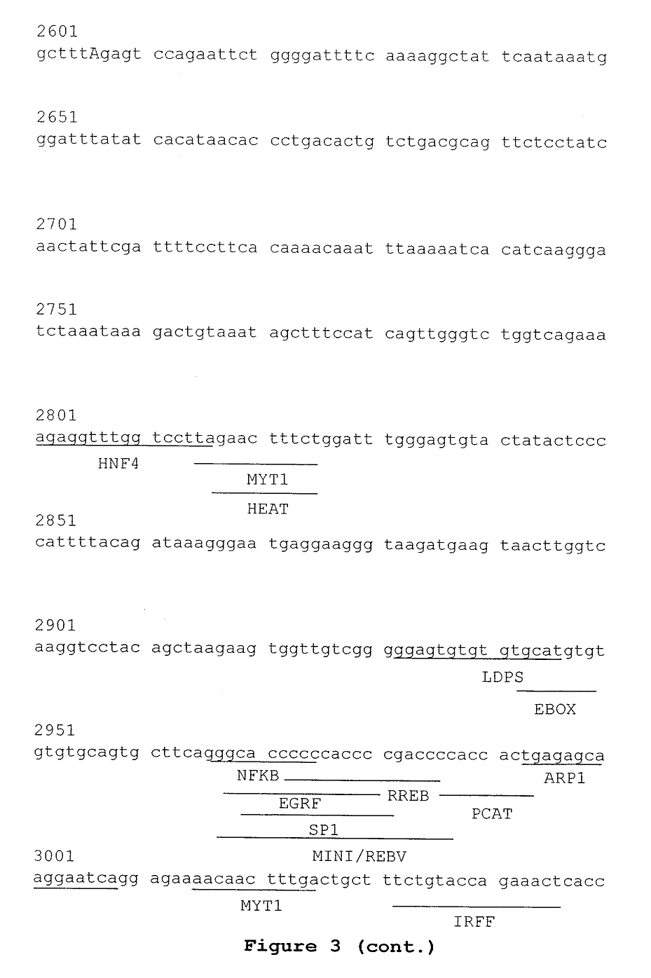

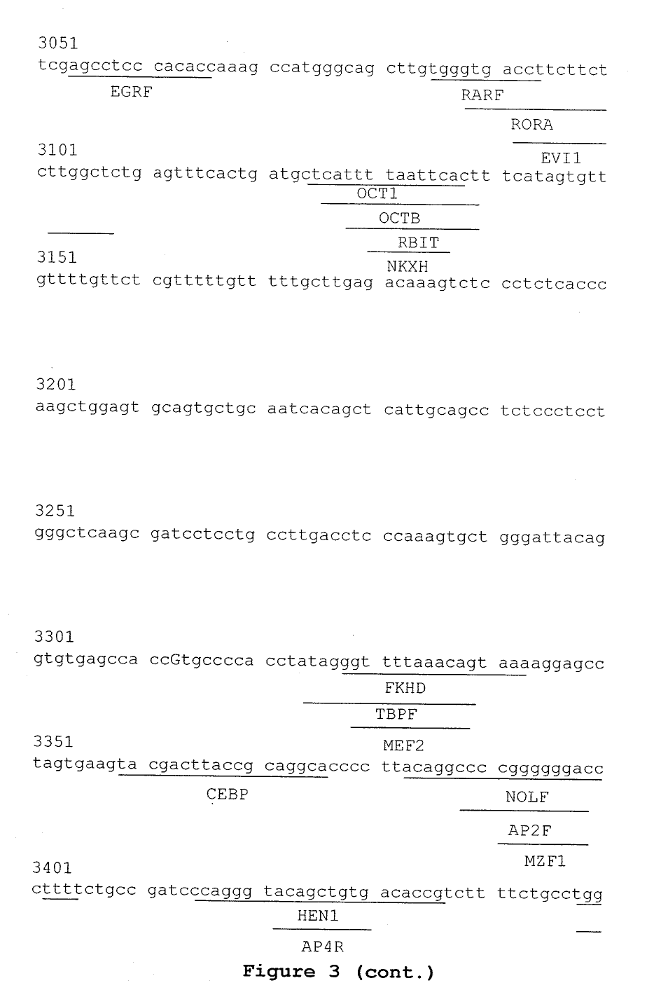

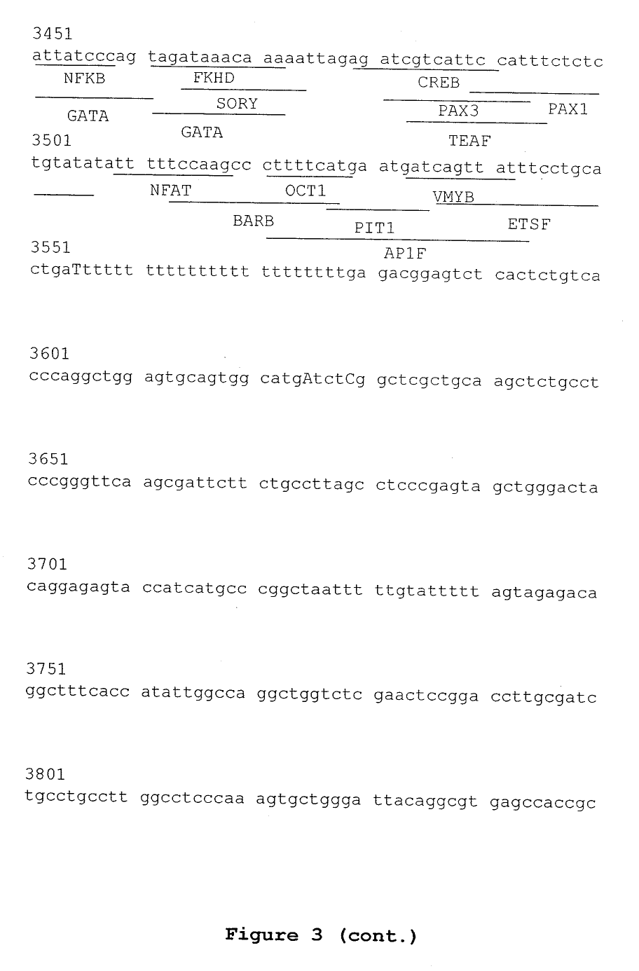

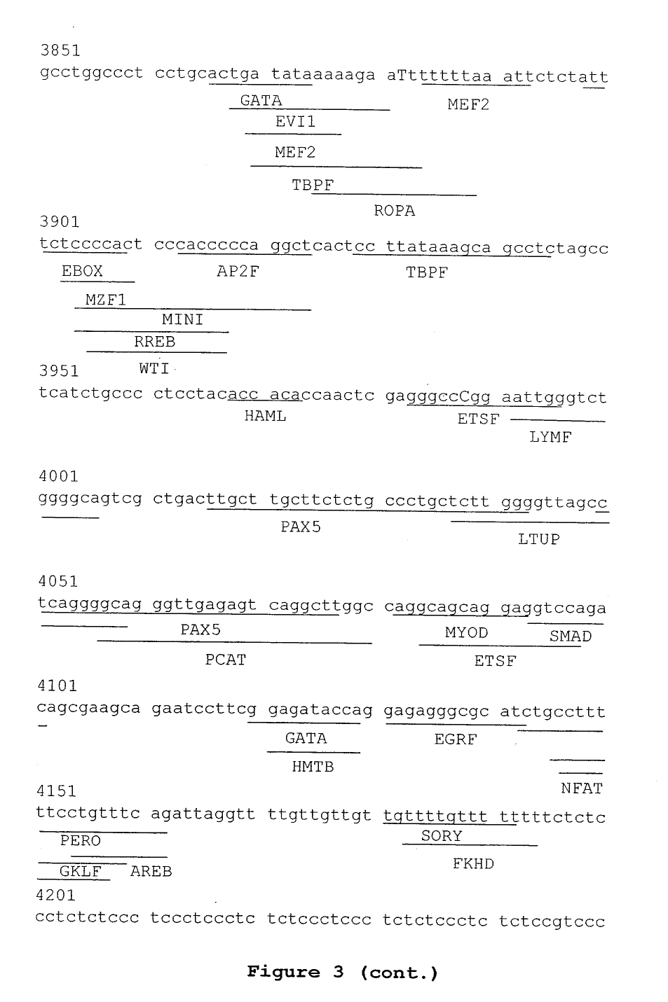

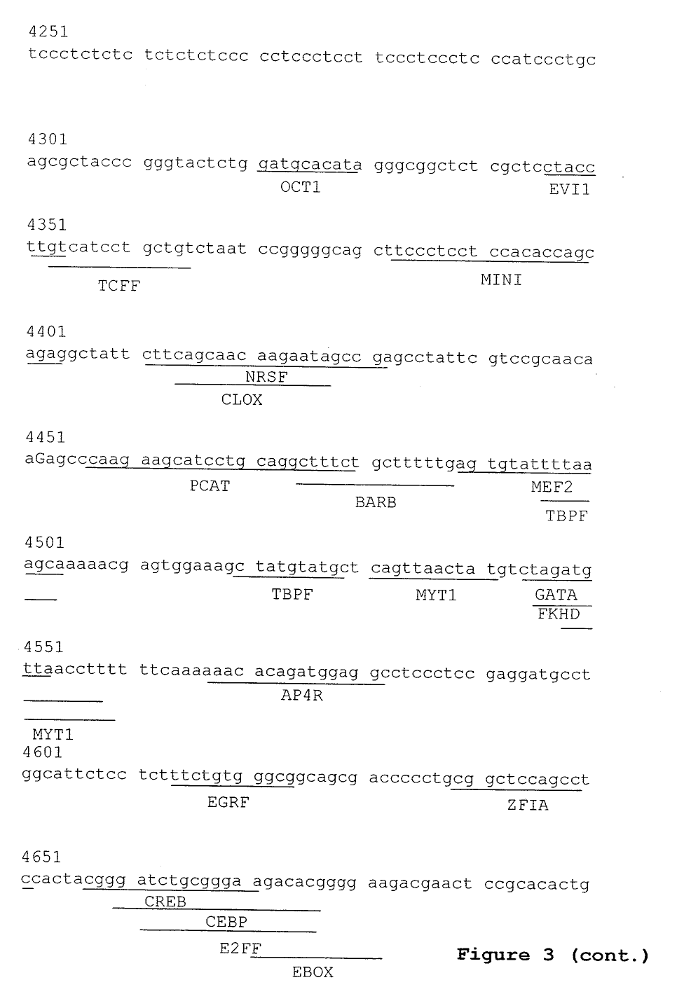

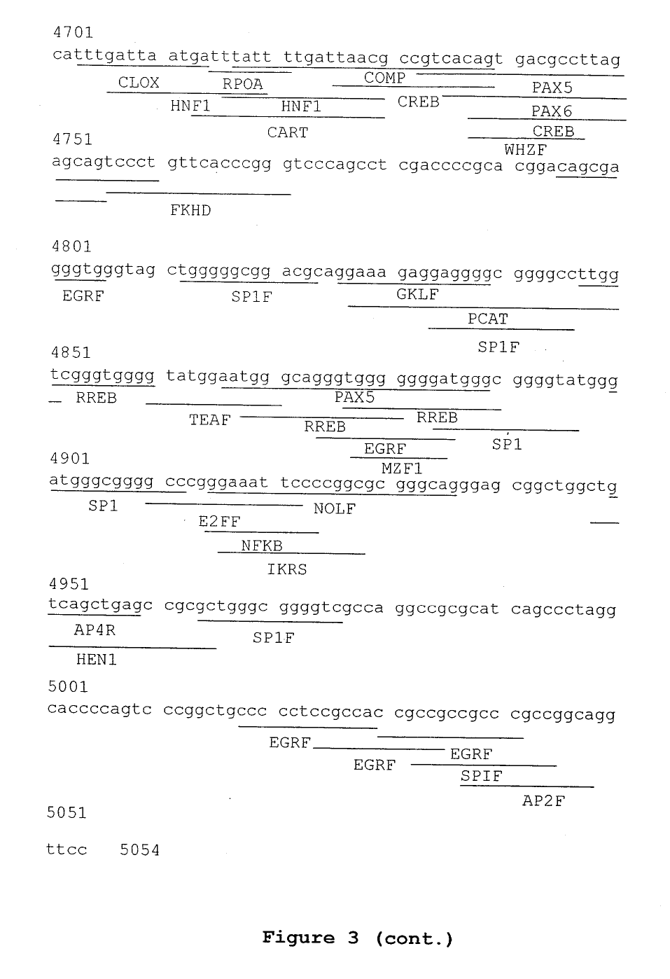

Sequence comparisons of the optineurin promoter region identify a number of DNA motifs (cis elements) and regulatory regions, which are listed below in Table 2. Selected motifs, regulatory regions, and SNPs are shown in FIG. 3. Table 2 contains data obtained by analyzing the optineurin promoter sequence (SEQ ID NO: 1) with MatInspector, which is a software tool that locates transcription factor binding sites in DNA sequences (Quandt et al., Nucleic Acid Research 23: 4878 (1995)). MatInspector itself, and a full description of the terminology used in Table 2 (e.g., family, matrix, core similarity, matrix similarity) may be obtained from Genomatrix Software GmbH (München, Germany or www.genomatix.de).

TABLE 2

| NAME OF FAMILY/MATRIX | FURTHER INFORMATION | POSITION | STRAND | CORE SIM. | MATRIX SIM. | SEQUENCE | SEQ. ID NO: |
|---|---|---|---|---|---|---|---|
| OCTB/TST1.01 | POU-factor Tst-1/Oct-6 | 10-24 | (−) | 1.000 | 0.877 | cagcAATTccacttc | 3 |
| AP1F/TCF11MAFG.01 | TCF11/MafG heterodimers, | 14-35 | (−) | 1.000 | 0.936 | atgataTGACccagcaattcca | 4 |
| | binding to sublcass of | | | | | | |
| | AP1 sites | | | | | | |
| GATA/GATA.01 | GATA binding site | 24-34 | (−) | 0.868 | 0.944 | tGATATgaccc | 5 |
| | (consensus) | | | | | | |
| EV11/EV11.05 | ectopic viral integration | 29-39 | (−) | 1.000 | 0.830 | agttatGATAt | 6 |
| | site 1 encoded factor | | | | | | |
| FKHD/FREAC2.01 | Fork head RElated | 39-54 | (−) | 1.000 | 0.891 | gaaagtTAAAcagaga | 7 |
| | Activator-2 | | | | | | |
| IRFF/IRF1.01 | interferon regulatory | 43-55 | (−) | 0.765 | 0.852 | ggaaagtTAAAca | 8 |
| | factor 1 | | | | | | |
| MYT1/MYT1.02 | MyT1 zinc finger tran- | 45-55 | (−) | 1.000 | 0.881 | ggaAAGTtaaa | 9 |
| | scription factor | | | | | | |
| | involved in primary | | | | | | |
| | neurogenesis | | | | | | |
| XBBF/M1F1.01 | M1BP-1/RFX1 complex | 47-64 | (−) | 0.850 | 0.768 | gagttccttgGAAAgtta | 10 |
| NFAT/NFAT.01 | Nuclear factor of | 48-59 | (−) | 1.000 | 0.951 | ccttgGAAAgtt | 11 |
| | activated T-cells | | | | | | |
| IKRS/IK3.01 | Ikaros 3, potential | 66-78 | (+) | 1.000 | 0.847 | tcctcGGAAtatt | 12 |
| | regulator of | | | | | | |
| | lymphocyte | | | | | | |
| | differentiation | | | | | | |
| OCTP/OCT1P.01 | octamer-binding factor | 67-81 | (−) | 0.980 | 0.895 | ccaaatATTCcgagg | 13 |
| | 1, POU-specific domain | | | | | | |
| PCAT/CAAT.01 | cellular and viral CCAAT | 79-90 | (+) | 0.847 | 0.904 | tggaaCCAGtga | 14 |
| | box | | | | | | |
| AP1F/AP1.01 | AP1 binding site | 95-103 | (−) | 0.917 | 0.955 | tTGATTCAg | 15 |
| BARB/BARBIE.01 | barbiturate-inducible | 103-117 | (+) | 1.000 | 0.873 | aactAAAGctgagac | 16 |
| | element | | | | | | |
| PERO/PPARA.01 | PPAR/RXR heterodimers | 106-125 | (+) | 1.000 | 0.713 | taaagctgagacAAAGtcca | 17 |
| AP1F/NFE2.01 | NF-E2p45 | 109-119 | (−) | 1.000 | 0.865 | ttgtcTCAGct | 18 |
| HNF4/HNF4.01 | Hepatic nuclear factor | 113-126 | (+) | 1.000 | 0.861 | gagaCAAAgtccag | 19 |
| | 4 | | | | | | |
| SMAD/SMAD3/01 | Smad 3 transcription | 121-128 | (−) | 1.000 | 0.996 | GTCTggac | 20 |
| | factor involved in | | | | | | |
| | TGF-beta signaling | | | | | | |
| RORA/RORA1.01 | RAR-related orphan | 125-137 | (+) | 1.000 | 0.945 | agaccaaGGTCaa | 21 |
| | receptor alpha 1 | | | | | | |
| SF1F/SF1.01 | SF1 steroidogenic factor | 128-136 | (+) | 1.000 | 0.988 | ccAAGGtca | 22 |
| | 1 | | | | | | |
| AP4R/TAL1ALPHAE47.01 | Tal-1alpha/E47 heterodimer | 141-156 | (+) | 1.000 | 0.888 | tagggCAGAtgattca | 23 |
| AP1F/AP1.01 | AP1 binding site | 149-157 | (−) | 0.934 | 0.960 | aTGAATCAt | 24 |
| PIT1/PIT1.01 | Pit1, GHF-1 pituitary | 152-161 | (+) | 0.871 | 0.872 | attcATGCag | 25 |
| | specific pou domain | | | | | | |
| | transcription factor | | | | | | |
| MINI/MUSCLE_IN1.03 | Muscle Initiator Sequence | 157-177 | (+) | 0.862 | 0.887 | tgcagcgacCACAccagtggc | 26 |
| HAML/AML1.01 | runt-factor AML-1 | 164-169 | (−) | 1.000 | 1.000 | tgTGGT | 27 |
| OZAZG/ROAZ.01 | Rat C2H2 Zn finger protein | 195-210 | (−) | 0.750 | 0.813 | ctgCAGCaaagggtgt | 28 |
| | involved in olfactory | | | | | | |
| | neuronal differentiation | | | | | | |
| MZF1/MZF1.01 | MZF1 | 214-221 | (−) | 1.000 | 0.971 | gttGGGGa | 29 |
| ETSF/ETS1.01 | c-Ets-1 binding site | 232-246 | (+) | 1.000 | 0.928 | ccaGGAActggtttc | 30 |
| RPOA/DTYPEPA.01 | PolyA signal of D-type | 242-251 | (−) | 1.000 | 0.834 | tCCATgaaac | 31 |
| | LTRs | | | | | | |
| STAT/STAT.01 | signal transducers and | 244-252 | (+) | 1.000 | 0.912 | ttcatGGAA | 32 |
| | activators of tran- | | | | | | |
| | scription | | | | | | |
| MYT1/MYT1.01 | MyT1 zinc finger tran- | 251-262 | (−) | 0.750 | 0.756 | aaAAATtgtctt | 33 |
| | scription factor involved | | | | | | |
| | in primary neurogenesis | | | | | | |
| NFAT/NFAT.01 | Nuclear factor of | 257-268 | (−) | 1.000 | 0.978 | ccatgGAAAaat | 34 |
| | activated T-cells | | | | | | |
| SRFF/SRF.03 | serum responsive factor | 259-273 | (−) | 0.819 | 0.842 | aCCATCcatggaaaa | 35 |
| CLOX/CDPCR3HD.01 | cut-like homeodomain | 264-273 | (+) | 0.929 | 0.936 | catgGATGgt | 36 |
| | protein | | | | | | |
| MINI/MUSCLEIINI.03 | Muscle Initiator Sequence | 270-290 | (−) | 1.000 | 0.862 | ccaccccccCACCcaccacca | 37 |
| R.REB/RREB1.01 | Ras-responsive element | 271-284 | (−) | 1.000 | 0.813 | cCCCAcccaccacc | 38 |
| | binding protein 1 | | | | | | |
| SP1F/SP1.01 | stimulating protein 1 SP1, | 274-286 | (+) | 0.819 | 0.890 | ggtgGGTGggggg | 39 |
| | ubiquitous zinc finger | | | | | | |
| | transcription factor | | | | | | |
| EGRF/WT1.01 | Wilms Tumor Suppressor | 277-289 | (+) | 1.000 | 0.937 | gggTGGGggggtg | 40 |
| RREB/RREB1.01 | Ras-responsive element | 285-298 | (−) | 1.000 | 0.851 | tCCCAaaaccaccc | 41 |
| | binding protein 1 | | | | | | |
| SEF1/SEF1.01 | SEF1 binding site | 310-328 | (−) | 0.809 | 0.686 | tgcctgatgaTCTGAggtg | 42 |
| PAX6/PAX6.01 | Pax-6 paired domain | 317-337 | (+) | 0.754 | 0.752 | gatcatcAGGCattagagtct | 43 |
| | protein | | | | | | |
| PDX1/PDX1.01 | Pdx1 (IDX1/IPFI) | 322-340 | (−) | 1.000 | 0.784 | atgagactcTAATgcctga | 44 |
| | pancreatic and intestinal | | | | | | |
| | homeodomain TF | | | | | | |
| AHRR/AHRARNT.01 | aryl hydrocarbon | 344-359 | (−) | 1.000 | 0.937 | tctaggttgCGTGctt | 45 |
| | receptor/Arnt | | | | | | |
| | heterodimers | | | | | | |
| FKHD/XFD3.01 | Xenopus fork head domain | 370-383 | (−) | 1.000 | 0.852 | attgtcAACAgaac | 46 |
| | factor 3 | | | | | | |
| SORY/SOX9.01 | SOX (SRY-related HMG box) | 374-387 | (+) | 1.000 | 0.906 | tgttgaCAAlaggg | 47 |
| CREB/TAXCREB.01 | Tax/CREB complex | 383-397 | (+) | 0.784 | 0.838 | tagggtTCACgctcc | 48 |
| PAX6/PAX6.01 | Pax-6 paired domain | 384-404 | (+) | 1.000 | 0.766 | agggttcACGCtcctatgaaa | 49 |
| | protein | | | | | | |
| E2FF/E2F.03 | E2F, involved in cell | 384-396 | (−) | 0.774 | 0.773 | gagCGTGaaccct | 50 |
| | cycle regulation, | | | | | | |
| | interacts with Rb 107 | | | | | | |
| | protein | | | | | | |
| AHRR/AHRARNT.01 | aryl hydrocarbon | 387-402 | (−) | 1.000 | 0.900 | tcataggagCGTGaac | 51 |
| | receptor/Amt | | | | | | |
| | heterodimers | | | | | | |
| OCT1/OCT1.05 | octamer-binding factor 1 | 402-415 | (−) | 0.888 | 0.903 | ctgcattagATTTt | 52 |
| AP4R/AP4.03 | activator protein 4 | 408-425 | (+) | 1.000 | 0.831 | taatgCAGCtgctgatct | 53 |
| MYOD/MYF5.01 | Myf5 myogenic bHLH protein | 410-421 | (+) | 1.000 | 0.948 | atgCAGCtgctg | 54 |
| SP1F/GC.01 | GC box elements | 429-442 | (+) | 1.000 | 0.903 | aagaGGCGgagctt | 55 |
| EGRF/WT1.01 | Wilms Tumor Suppressor | 452-464 | (−) | 1.000 | 0.892 | gggTGGGtgagca | 56 |
| VMYB/VMYB.02 | v-Myb | 462-470 | (−) | 1.000 | 0.951 | agcAACGgg | 57 |
| PERO/PPARA.01 | PPAR/RXR heterodimers | 494-513 | (+) | 0.807 | 0.695 | tcctgagaggccACAGgcca | 58 |
| HNF4/HNF4.01 | Hepatic nuclear factor 4 | 501-514 | (+) | 0.750 | 0.848 | aggcCACAggccag | 59 |
| B2TF/E2.01 | BPV bovine papilloma virus | 522-537 | (−) | 0.852 | 0.878 | aaaccccgggTGGTga | 60 |
| | regulator E2 | | | | | | |
| RREB/RREB1.01 | Ras-responsive element | 528-541 | (−) | 1.000 | 0.827 | cCCCAaaccccggg | 61 |
| | binding protein 1 | | | | | | |
| GKLF/GKLF.01 | gut-enriched Krueppel-like | 543-556 | (−) | 0.950 | 0.916 | caataaagcaGGGG | 62 |
| | factor | | | | | | |
| CLOX/CDP.01 | cut-like homeodomain | 546-557 | (−) | 1.000 | 0.780 | ccAATAaagcag | 63 |
| | protein | | | | | | |
| RPOA/LPOLYA.01 | Lentiviral Poly A signal | 549-556 | (−) | 1.000 | 1.000 | cAATAAAg | 64 |
| HOXF/HOX1-30.1 | Hox-1.3, vertebrate | 550-579 | (+) | 1.000 | 0.748 | tttattggacataATTAttaggtcgtgttc | 65 |
| | homeobox protein | | | | | | |
| ECAT/NFY.02 | nuclear factor Y | 550-560 | (−) | 1.000 | 0.914 | tgtCCAAtaaa | 66 |
| | (Y-box binding factor) | | | | | | |
| PCAT/CAAT.01 | cellular and viral CCAAT | 551-562 | (−) | 1.000 | 0.916 | tatgtCCAAtaa | 67 |
| | box | | | | | | |
| HMYO/S8.01 | S8 | 555-570 | (+) | 1.000 | 0.970 | tggacataATTAttag | 68 |
| NKXH/NKX25.02 | homeo domain factor | 559-566 | (+) | 0.944 | 0.950 | cATAAtta | 69 |
| | Nkx-2.5/Csx, tinman | | | | | | |
| | homolog low affinity sites | | | | | | |
| GREF/PRE.01 | Progesterone receptor | 560-586 | (+) | 1.000 | 0.881 | atattattaggtcgTGTTctttttgg | 70 |
| MEF2/MEF2.01 | myogenic enhancer factor 2 | 573-588 | (−) | 0.750 | 0.742 | cacCAAAaagaacacg | 71 |
| EBOX/USF.02 | upstream stimulating | 618-625 | (+) | 0.875 | 0.938 | cCACATgc | 72 |
| | factor | | | | | | |
| CDXF/CD2.01 | Cdx-2 mammalian caudal | 620-638 | (−) | 1.000 | 0.900 | ggtgaatTTTAtggcatgt | 73 |
| | related intestinal | | | | | | |
| | transcr. factor | | | | | | |
| MEF2/AMEF2.01 | myocyte eithancer factor | 623-640 | (+) | 1.000 | 0.817 | tgccaTAAAattcacccc | 74 |
| RPOA/DTYPEPA.01 | PolyA signal of D-type | 624-633 | (+) | 1.000 | 0.816 | gCCATaaaat | 75 |
| | LTRs | | | | | | |
| TBPF/TATA.02 | Mammalian C-type LTR TATA | 624-633 | (+) | 0.925 | 0.941 | gcCATAAAAt | 76 |
| | box | | | | | | |
| EBOX/SREBP1.02 | sterol regulatory element- | 632-642 | (+) | 1.000 | 0.832 | atTCACcccat | 77 |
| | binding protein 1 | | | | | | |
| PIT1/PIT1.01 | Pit1, GHF-1 pituitary | 649-658 | (−) | 0.820 | 0.905 | aatcATACat | 78 |
| | specific pou domain | | | | | | |
| | transcription factor | | | | | | |
| AP1F/AP1.01 | AP1 binding site | 653-661 | (−) | 0.934 | 0.960 | aTGAATCAt | 79 |
| HMYO/S8.01 | S8 | 662-677 | (+) | 1.000 | 0.969 | ggctttcaATTAcact | 80 |
| OCTB/TST1.01 | POU-factor Tst-1/Oct-6 | 665-679 | (+) | 1.000 | 0.902 | tttcAATTacactta | 81 |
| NKXH/NKX31.01 | prostate-specific | 670-682 | (−) | 1.000 | 0.892 | ttttAAGTgtaat | 82 |
| | homeodomain protein NKX3.1 | | | | | | |
| TBPF/ATATA.01 | Avian C-type LTR TATA box | 675-684 | (−) | 0.812 | 0.833 | cTTTTTAagt | 83 |
| MYT1/MYT1.01 | MyT1 zinc finger tran- | 679-689 | (+) | 1.000 | 0.899 | aaaAAGTtgta | 84 |
| | scription factor involved | | | | | | |
| | in primary neurogenesis | | | | | | |
| CDXF/CDX2.01 | Cdx-2 mammalian caudal | 680-698 | (−) | 1.000 | 0.835 | tgatggtTTTAcaactttt | 85 |
| | related intestinal | | | | | | |
| | transcr. factor | | | | | | |
| HOXF/HOX1-3.01 | Hox-1.3, vertebrate | 685-714 | (+) | 1.000 | 0.773 | ttgtaaaaccatcATTAcaattcaaattta | 86 |
| | homeobox protein | | | | | | |
| PDX1/PDX1.01 | Pdx1 (IDX1/IPF1) | 687-705 | (+) | 0.782 | 0.805 | gtaaaaccaTCATtacaat | 87 |
| | pancreatic and intestinal | | | | | | |
| | homeodomain TF | | | | | | |
| SORY/SOX5.01 | Sox-5 | 698-705 | (+) | 1.000 | 0.862 | attaCAATtc | 88 |
| RPOA/APOLYA.01 | Avian C-type LTR PolyA | 702-716 | (−) | 0.853 | 0.713 | ACTAAAtttgaattg | 89 |
| | signal | | | | | | |
| MYT1/MYT1.01 | MyT1 zinc finger tran- | 703-714 | (−) | 0.750 | 0.756 | taAATTtgaatt | 90 |
| | scription factor involved | | | | | | |
| | in primary neurogenesis | | | | | | |
| OCT1/OCT1.02 | octamer-binding factor 1 | 718-727 | (−) | 0.755 | 0.864 | gATGGaaata | 91 |
| RREB/RREB1.01 | Ras-responsive element | 731-744 | (+) | 1.000 | 0.898 | cCCCAaaaatcccc | 92 |
| | binding protein 1 | | | | | | |
| MZF1/MZF1.01 | MZF1 | 740-747 | (−) | 1.000 | 0.975 | cgaGGGGa | 93 |
| PCAT/ACAAT.01 | Avian C-type LTR CCAAT | 771-779 | (+) | 0.825 | 0.879 | ccCCCAAtt | 94 |
| | box | | | | | | |
| STAT/STAT3.01 | signal transducer and | 773-793 (+) | 0.750 | 0.735 | cccaatTTCAggcaactactg | 96 | |
| | activator of transcription | | | | | | |
| | 3 | | | | | | |
| GF11/GF11.01 | growth factor independence | 786-809 | (−) | 1.000 | 0.938 | aagacagaAAtcagaccagtagtt | 96 |
| | 1 zinic finger protein | | | | | | |
| | acts as transcriptional | | | | | | |
| 1RFF/1SRE.01 | interferon-stimulated | 814-828 | (−) | 1.000 | 0.825 | cagaaaagGAAAgta | 97 |
| | response element | | | | | | |
| NFAT/NFAT.01 | Nuclear factor of | 814-825 | (−) | 1.000 | 0.953 | aaaagGAAAgta | 98 |
| | activated T-cells | | | | | | |
| SRFF/SRF.02 | serum response factor | 818-831 | (−) | 0.847 | 0.895 | gtCCAGaaaaggaa | 99 |
| RPOA/DTYPEPA.01 | PolyA signal of D-type | 832-841 | (−) | 0.750 | 0.797 | tACATtaaat | 100 |
| | LTRs | | | | | | |
| OCTP/OCT1P.01 | octamer-binding factor 1, | 834-848 | (−) | 0.849 | 0.863 | ctccatATACattaa | 101 |
| | POU-specific domain | | | | | | |
| XSEC/STAF.01 | Se-Cys tRNA gene tran- | 862-883 | (−) | 0.778 | 0.765 | gctaCCCCagatgccaaagact | 102 |
| | scription activating | | | | | | |
| | factor | | | | | | |
| LYMF/TH1E47.01 | Thing 1/E47 heterodimer, | 866-881 | (+) | 1.000 | 0.914 | tttggcatCTGGggta | 103 |
| | TH1 bHLH member specific | | | | | | |
| | expression in a variety of | | | | | | |
| | embryonic tissues | | | | | | |
| HOXF/HOX1-3.01 | Hox-1.3, vertebrate | 881-910 | (+) | 1.000 | 0.783 | agcaagtacgaatATTAgtctaccacctca | 104 |
| | homeobox protein | | | | | | |
| OCTP/OCT1P.01 | octamer-binding factor 1, | 885-899 | (−) | 0.980 | 0.909 | actaatATTCgtact | 105 |
| | POU-specific domain | | | | | | |
| SEF1/SEF1.01 | SEF1 binding site | 904-922 | (−) | 0.809 | 0.684 | tttatgtgcaTCTGAggtg | 106 |
| CDXF/CDX2.01 | Cdx-2 mammalian caudal | 911-929 | (−) | 1.000 | 0.863 | taatattTTTAtgtgcatc | 107 |
| | related intestinal | | | | | | |
| | transcr. factor | | | | | | |
| OCT1/OCT1.05 | Octamer-binding factor 1 | 915-928 | (−) | 1.000 | 0.891 | aatatttttATGTg | 108 |
| OCT1/OCT1.05 | Octamer-binding factor 1 | 922-935 | (+) | 0.944 | 0.894 | aaatattacATATc | 109 |
| CREB/E4BP4.01 | E4P4, bZIP domain, tran- | 925-936 | (−) | 1.000 | 0.878 | agatatGTAAta | 110 |
| | scriptional repressor | | | | | | |
| GATA/GATA.01 | GATA binding site | 926-936 | (−) | 0.868 | 0.942 | agatatGTAAtaat | 111 |
| | (consensus) | | | | | | |
| VBPF/VBP.01 | PAR-type chicken | 926-935 | (+) | 1.000 | 0.889 | aTTACatatc | 112 |
| | vitellogenin | | | | | | |
| | promotor-binding protein | | | | | | |
| EV11/EV11.03 | ectopic viral integration | 932-946 | (−) | 0.800 | 0.927 | aGAAAagaaaagata | 113 |
| | site 1 encoded factor | | | | | | |
| NFAT/NFAT.01 | Nuclear factor of | 944-955 | (−) | 1.000 | 0.951 | ggaagGAAAaga | 114 |
| | activated T-cells | | | | | | |
| ETSF/ETS1.01 | c-Ets-1 binding site | 981-995 | (−) | 1.000 | 0.909 | gaaGGAAgtagagag | 115 |
| YY1F/YY1.01 | Yin and Yang 1 | 1084-1103 | (+) | 1.000 | 0.871 | gtggcaCCATcttggctcag | 116 |
| MYOF/NF1.01 | nuclear factor 1 | 1093-1110 | (+) | 1.000 | 0.940 | tctTGGCtcagcgcaacc | 117 |
| XBBF/RFX1.01 | X-box binding protein RFX1 | 1095-1111 | (+) | 1.000 | 0.880 | ttggctcagcGCAAcct | 118 |
| AP1F/NFE2.01 | NF-E2 p45 | 1095-1105 | (+) | 1.000 | 0.865 | ttggcTCAGcg | 119 |
| BRAC/BRACH.01 | Brachyury | 1145-1168 | (+) | 0.750 | 0.693 | agcctctcaagtAGCTgagattac | 120 |
| TTFF/TTF1.01 | Thyroid transcription | 1147-1160 | (+) | 1.000 | 0.942 | cctctCAAGtagct | 121 |
| | factor-1 (TTF1) binding | | | | | | |
| | site | | | | | | |
| AP1F/BEL1.01 | Bel-1 similar region | 1153-1180 | (−) | 0.919 | 0.810 | tggtgcgtgcctgtaatCTCAGctactt | 122 |
| GATA/GATA3.01 | GATA binding factor 3 | 1160-1169 | (+) | 0.824 | 0.906 | tgaGATTaca | 123 |
| AHRR/AHRARNT.01 | aryl hydrocarbon | 1169-1184 | (−) | 1.000 | 0.937 | gtagtggtgCGTGcct | 124 |
| | receptor/Amt heterodimers | | | | | | |
| MEF2/HMEF2.01 | myocyte enhancer factor | 1189-1204 | (−) | 1.000 | 0.762 | atataaAAATtagcca | 125 |
| HNF1/HNF1.02 | Hepatic nuclear factor 1 | 1190-1206 | (+) | 0.859 | 0.755 | gGCTAatttttatattt | 126 |
| TBPF/TATA.01 | cellular and viral TATA | 1190-1204 | (−) | 1.000 | 0.951 | ataTAAAaattagcc | 127 |
| | box elements | | | | | | |
| FKHD/XFD2.01 | Xenopus fork head domain | 1192-1205 | (−) | 1.000 | 0.905 | aataTAAAaattag | 128 |
| | factor 2 | | | | | | |
| OCT1/OCT1.05 | octamer-binding factor 1 | 1192-1205 | (+) | 0.944 | 0.917 | ctaatttttATATt | 129 |
| MEF2/RSRFC4.02 | related to serum response | 1197-1213 | (−) | 1.000 | 0.885 | ctactaaaAATAtaaaa | 130 |
| | factor, C4 | | | | | | |
| GATA/LMO2COM.02 | complex of Lmo2 bound to | 1213-1221 | (+) | 1.000 | 0.992 | gaGATAggg | 131 |
| | Tal-1, E2A proteins; and | | | | | | |
| | GATA-1, half-site 2 | | | | | | |
| AREB/AREB6.04 | AREB6 (Atplal regulatory | 1219-1227 | (+) | 1.000 | 0.970 | ggGTTTcac | 132 |
| | element binding factor 6) | | | | | | |
| CREB/HLF.01 | hepatic leukemia factor | 1221-1230 | (+) | 0.770 | 0.832 | GTTTcaccat | 133 |
| ARP1/ARP1.01 | apolipoprotein AI | 1248-1263 | (+) | 0.826 | 0.842 | tgaactCCTGacctca | 134 |
| | regulatory protein 1 | | | | | | |
| T3RH/T3R.01 | vErbA, viral homolog of | 1251-1266 | (−) | 1.000 | 0.924 | gtttgaggtcaggagt | 135 |
| | thyroid hormone receptor | | | | | | |
| | alpha 1 | | | | | | |
| RARF/RAR.01 | Retinoic acid receptor, | 1252-1261 | (−) | 0.897 | 0.961 | aggTCAGgag | 136 |
| | member of nuclear | | | | | | |
| | receptors | | | | | | |
| RORA/RORA1.01 | RAR-related orphan | 1255-1267 | (−) | 1.000 | 0.933 | cgtttgaGGTCag | 137 |
| | receptor alpha 1 | | | | | | |
| CREB/CREBP1CJUN.01 | CRE-binding protein | 1256-1263 | (+) | 0.769 | 0.885 | tgACCTca | 138 |
| | 1/c-Jun heterodimer | | | | | | |
| LYMF/LYF1.01 | LyE-1, enriched in B and T | 1270-1278 | (−) | 1.000 | 0.988 | tttGGGAgg | 139 |
| | lymphocytes | | | | | | |
| HOBO/HOGNESS.01 | Imperfect Hogness/Goldberg | 1277-1308 | (−) | 0.764 | 0.922 | ggcggtggctcacgccTGlAatcccagcactt | 140 |
| | Box | | | | | | |
| IKRS/JK2.01 | Ikaros 2, potential | 1280-1291 | (+) | 1.000 | 0.960 | tgctGGGAttac | 141 |
| | regulator of lymphocyte | | | | | | |
| | differentiation | | | | | | |
| CREB/TAXCREB.01 | Tax/CREB complex | 1291-1305 | (−) | 0.784 | 0.806 | ggtggcTCACgcctg | 142 |
| SP1F/SP1.01 | stimulating protein 1 SP1, | 1300-1312 | (−) | 1.000 | 0.881 | ccagGGCGgtggc | 143 |
| | ubiquitous zinc finger | | | | | | |
| | transcription factor | | | | | | |
| FKHD/FREAC2.01 | Fork head Related | 1312-1327 | (−) | 1.000 | 0.841 | agaaagTAAAgaggcc | 144 |
| | Activator-2 | | | | | | |
| TBPF/MTATA.01 | Muscle TATA box | 1324-1340 | (+) | 1.000 | 0.855 | ttcttTAAAcccagttc | 145 |
| MEF2/MEF2.05 | MEF2 | 1325-1334 | (−) | 1.000 | 0.984 | ggttTAAAga | 146 |
| XBBF/MIF1.01 | MIBP-1/RFX1 complex | 1345-1362 | (+) | 0.850 | 0.764 | ggggtgtacgGAAAccta | 147 |
| AREB/AREB6.04 | AREB6 (Atpial regulatory | 1353-1361 | (−) | 1.000 | 0.974 | agGTTTccg | 148 |
| | element binding factor 6) | | | | | | |
| E2FF/E2F.02 | E2F, involved in cell | 1364-1371 | (−) | 1.000 | 0.849 | gcccGAAA | 149 |
| | cycle regulation, | | | | | | |
| | interacts with Rb p107 | | | | | | |
| | protein | | | | | | |
| LYMF/TH1E47.01 | Thing 1/E47 heterodimer, | 1375-1390 | (+) | 1.000 | 0.928 | actggggtCTGGagag | 150 |
| | TH1 Bhlh member specific | | | | | | |
| | expression in a variety of | | | | | | |
| | embryonic tissues | | | | | | |
| MZF1/MFZF1.01 | MZF1 | 1387-1394 | (+) | 1.000 | 0.986 | agaGGGGa | 151 |
| OCT1/OCT1.02 | octamer-binding factor 1 | 1413-1422 | (+) | 1.000 | 0.943 | cATGCaaaac | 152 |
| PAX5/PAX9.01 | zebrafish PAX9 binding | 1438-1461 | (+) | 0.933 | 0.774 | ggtaCCCAttgaagtaagggccat | 153 |
| | sites | | | | | | |
| RPOA/DTYPEPA.01 | PolyA signal of D-type | 1442-1451 | (+) | 1.000 | 0.779 | cCCATtgaag | 154 |
| | LTRs | | | | | | |
| VBPF/VBP.01 | PAR-type chicken | 1446-1455 | (−) | 1.000 | 0.862 | cTTACttcaa | 155 |
| | vitellogenin promoter- | | | | | | |
| | binding protein | | | | | | |
| CREB/CREBP1.01 | cAMP-responsive element | 1447-1454 | (−) | 0.766 | 0.820 | ttACTTca | 156 |
| | binding protein 1 | | | | | | |
| RPOA/LPOLYA.01 | Lentiviral Poly A signal | 1460-1467 | (−) | 1.000 | 0.963 | aAATAAAt | 157 |
| XBBF/RFX1.01 | X-box binding protein RFX1 | 1467-1483 | (+) | 1.000 | 0.883 | tttcagcccaGCAAcat | 158 |
| HOXF/HOX1-3.01 | Hox-1.3, vertebrate | 1487-1516 | (+) | 1.000 | 0.787 | cactgataccctcATTAtcaaatggttctt | 159 |
| | homeobox protein | | | | | | |
| GATA/GATA1.03 | GATA-binding factor 1 | 1497-1509 | (−) | 1.000 | 0.943 | atttGATAatgag | 160 |
| IKRS/IK3.01 | Ikaros 3, potential | 1516-1528 | (+) | 1.000 | 0.840 | tctagGGAAcagt | 161 |
| | regulator of lymphocyte | | | | | | |
| | differentiation | | | | | | |
| NFAT/NFAT.01 | Nuclear factor of | 1534-1545 | (−) | 1.000 | 0.970 | cattgGAAAcag | 162 |
| | activated T-cells | | | | | | |
| AREB/AREB6.04 | AREB6 (Atplal regulatory | 1534-1542 | (+) | 1.000 | 0.991 | ctGTTTcca | 163 |
| | element binding factor 6) | | | | | | |
| ECAT/NFY.02 | Nuclear factor Y | 1537-1547 | (+) | 1.000 | 0.917 | tttCCAAtgac | 164 |
| | (Y-box binding factor) | | | | | | |
| CBBP/CEBP.02 | C/EBP binding site | 1570-1587 | (−) | 0.769 | 0.854 | ggactttgGGAACctccc | 165 |
| NFKB/CREL.01 | c-Rel | 1570-1579 | (+) | 1.000 | 0.940 | gggaggTTCC | 166 |
| IKRS/1K2.01 | Ikaros 2, potential | 1573-1584 | (−) | 1.000 | 0.966 | ctttGGGAacct | 167 |
| | regulator of lymphocyte | | | | | | |
| | differentiation | | | | | | |
| XSEC/STAF.01 | Se-Cys tRNA gene tran- | 1574-1595 | (+) | 1.000 | 0.781 | ggttCCCAaagtccagtaggtg | 168 |
| | scription activating | | | | | | |
| | factor | | | | | | |
| SMAD/SMAD3.01 | Smad3 transcription factor | 1617-1624 | (+) | 1.000 | 0.997 | GTCTgggt | 169 |
| | involved in TGF-beta | | | | | | |
| | signaling | | | | | | |
| CP2F/CP2.01 | CP2 | 1619-1629 | (−) | 1.000 | 0.915 | gcagcacCCAG | 170 |
| PAX6/PAX6.01 | Pax-6 paired domain | 1630-1650 | (−) | 0.773 | 0.753 | aggactcAAGCctcagtccct | 171 |
| | protein | | | | | | |
| ARP1/ARP1.01 | Apolipoprotein AI | 1643-1658 | (+) | 1.000 | 0.829 | tgagtcCTTGatgctc | 172 |
| | regulatory protein 1 | | | | | | |
| RPAD/PADS.01 | Mammalian C-type LTR Poly | 1661-1669 | (−) | 1.000 | 0.936 | gGTGGTctt | 173 |
| | A downstream element | | | | | | |
| ECAT/NFY.01 | Nuclear factor Y | 1680-1695 | (+) | 1.000 | 0.899 | tcctcCCAAtctgggg | 174 |
| | (Y-box binding factor) | | | | | | |
| SRFF/SRF.02 | Serum response factor | 1682-1695 | (−) | 0.847 | 0.868 | ccCCAGatrgggag | 175 |
| SP1F/SP1.01 | Stimulating protein 1 SP1, | 1691-1703 | (+) | 1.000 | 0.967 | tgggGGCGgggga | 176 |
| | ubiquitous zinc finger | | | | | | |
| | transcription factor | | | | | | |
| EGRE/EGR1.01 | Egr-1/Kirox-24/NGFI-A | 1694-1705 | (+) | 0.830 | 0.813 | gggcgggGGAGt | 177 |
| | intermediate-early gene | | | | | | |
| | product | | | | | | |
| AP1F/AP1.03 | Activator protein 1 | 1699-1709 | (−) | 1.000 | 0.935 | agTGACtcccc | 178 |
| CMYB/CMYB.01 | c-Myb, important in | 1714-1731 | (−) | 1.000 | 0.942 | tttcacaacaGTTGgagg | 179 |
| | hematopoesis, cellular | | | | | | |
| | equivalent to avian myo- | | | | | | |
| | blastosis virus oncogene | | | | | | |
| | v-myb | | | | | | |
| VMYB/VMYB.02 | v-Myb | 1716-1724 | (+) | 0.819 | 0.895 | tccAACTgt | 180 |
| CEBP/CEBPB.01 | CCAAT/enhancer binding | 1721-1734 | (+) | 0.985 | 0.942 | ctgttgtGAAAgcc | 181 |
| | protein beta | | | | | | |
| MINI/MUSCLE_INI.02 | Muscle Initiator Sequence | 1733-1753 | (+) | 1.000 | 0.853 | cctccaccCCACccagctctg | 182 |
| EBOX/SREBP1.02 | Sterol regulatory element- | 1734-1744 | (+) | 0.750 | 0.838 | ctCCACcccac | 183 |
| | binding protein 1 | | | | | | |
| PAX5/PAX9.01 | Zebrafish PAX9 binding | 1736-1759 | (−) | 0.800 | 0.862 | aagaGCCAgagctgggtggggtgg | 184 |
| | sites | | | | | | |
| SP1F/GC.01 | GC box elements | 1736-1749 | (−) | 0.872 | 0.884 | gctgGGTGgggtgg | 185 |
| NFKB/CREL.01 | c-Rel | 1752-1761 | (+) | 1.000 | 0.909 | tggctcTTCC | 186 |
| ETSF/GABP.01 | GABP: GA binding protein | 1753-1764 | (−) | 1.000 | 0.872 | ggaGGAAgagcc | 187 |
| SEF1/SEF1.01 | SEF1 binding site | 1761-1779 | (+) | 0.809 | 0.777 | ctccaggacaTCTGGggta | 188 |
| AP4R/TALIALPHAE47.01 | Tal-1alpha/E47 heterodimer | 1764-1779 | (−) | 1.000 | 0.867 | tacccCAGAtgtcctg | 189 |
| REOA/POLYA.01 | Mammalian C-type LTR Poly | 1778-1795 | (−) | 0.822 | 0.823 | cAATACAtccatgatcta | 190 |
| | A signal | | | | | | |
| EVI1/EVI1.02 | Ectopic viral integration | 1814-1824 | (+) | 1.000 | 0.837 | agacAAGAaga | 191 |
| | site 1 encoded factor | | | | | | |
| CMYB/CMYB.01 | c-Myb, important in | 1836-1853 | (+) | 1.000 | 0.936 | tctaagagctGTTGccag | 192 |
| | hematopoesis, cellular | | | | | | |
| | equivalent to avian myo- | | | | | | |
| | blastosis virus oncogene | | | | | | |
| | v-myb | | | | | | |
| XBBF/RFX1.01 | X-box binding protein RFX1 | 1844-1860 | (−) | 1.000 | 0.922 | tggactcctgGCAAcag | 193 |
| MYOF/NF1.01 | Nuclear factor 1 | 1850-1867 | (−) | 1.000 | 0.959 | cgtTGGCtggactcctgg | 194 |
| EGRF/EGR3.01 | Early growth response gene | 1859-1870 | (−) | 1.000 | 0.795 | gaGCGTtggctg | 195 |
| | 3 product | | | | | | |
| NOLF/OLF1.01 | olfactory neuron-specific | 1879-1900 | (−) | 1.000 | 0.825 | aacgagTCCCtttgggcttcct | 196 |
| | factor | | | | | | |
| AREB/AREB6.04 | AREB6 (Atpla1 regulatory | 1907-1915 | (−) | 1.000 | 0.970 | ctGTTTgga | 197 |
| | element binding factor 6) | | | | | | |
| GREF/ARE.01 | Androgene receptor binding | 1929-1955 | (−) | 1.000 | 0.796 | gtttgatgttccttgTGTTccctttcc | 198 |
| | site | | | | | | |
| IRFF/IRF2.01 | Interferon regulatory | 1929-1941 | (+) | 0.750 | 0.803 | ggaaaggGAACac | 199 |
| | factor 2 | | | | | | |
| LDPS/LDSPOLYA.01 | Lentiviral Ply A down- | 1931-1946 | (−) | 0.862 | 0.923 | tccTTGTgttcccttt | 200 |
| | stream element | | | | | | |
| XBBF/RFX1.02 | X-box binding protein RFX1 | 1933-1950 | (+) | 0.881 | 0.904 | agggaacacaaGGAAcat | 201 |
| RPOA/DTYPEPA.01 | Poly A signal of D-type | 1946-1955 | (+) | 0.750 | 0.777 | aACATcaaac | 202 |
| | LTRs | | | | | | |
| IKRS/IK1.01 | Ikaros 1, potential | 977-1989 | (−) | 1.000 | 0.918 | gtgtGGGAaggtt | 203 |
| | regulator of lymphocyte | | | | | | |
| | differentiation | | | | | | |
| XSEC//STAF.02 | Se-Cys tRNA gene tran- | 979-1999 | (+) | 1.000 | 0.864 | ccttCCCAcactgctctacat | 204 |
| | scription activating | | | | | | |
| | factor | | | | | | |
| RPOA/DTYPEPA.01 | Poly A signal of D-type | 2006-2015 | (+) | 0.75 | 0.777 | aCCACaaaac | 205 |
| | LTRs | | | | | | |
| HAML/AML1.01 | runt-factor AML-1 | 2006-2011 | (−) | 1.000 | 1.000 | tgTGGT | 206 |
| HAML/AML1.01 | runt-factor AML-1 | 2014-2019 | (−) | 1.000 | 1.000 | tgTGGT | 207 |
| ECAT/NFY.03 | Nuclear factor Y | 2019-2032 | (+) | 0.777 | 0.847 | atcaACAAAtcagc | 208 |
| | (Y-box binding factor) | | | | | | |
| TBPF/ATATA.01 | Avian C-type LTR TATA BOX | 2046-2055 | (+) | 0.812 | 0.824 | tTATTTCagt | 209 |
| IRFF/IRF1.01 | interferon regulatory | 2047-2059 | (−) | 1.000 | 0.879 | aaaaactGAAAta | 210 |
| | factor 1 | | | | | | |
| VMYB/VMYB.01 | v-Myb | 2050-2059 | (−) | 0.876 | 0.910 | aaaAACTgaa | 211 |
| PAX6/PAX6.01 | Pax-6 paired domain | 2053-2073 | (+) | 0.754 | 0.751 | agtttttTCGCtgcatttaga | 212 |
| | protein | | | | | | |
| E2FF/E2F,02 | E2F involved in cell cycle | 2056-2063 | (−) | 0.857 | 0.866 | gcgaAAAA | 213 |
| | regulation, interacts with | | | | | | |
| | Rb p107 protein | | | | | | |
| PAX5/PAX9.01 | zebrafish PAX9 binding | 2079-2102 | (+) | 0.933 | 0.793 | tctaCCCAtggaagtgtcaggaa | 214 |
| | sites | | | | | | |
| MTF1/MTF-1.01 | Metal transcripton factor | 2087-2101 | (−) | 1.000 | 0.873 | tcctGCACacttcca | 215 |
| | 1, MRE | | | | | | |
| ETSF/ETS2.01 | c-Ets-2 binding site | 2095-2108 | (+) | 1.000 | 0.863 | tgcaGGAAgatgga | 216 |
| ZFIA/ZID.01 | zinc finger with inter- | 2100-2112 | (−) | 0.777 | 0.865 | tgACTCcatcttc | 217 |
| | action domain | | | | | | |
| AP1F/AP1F1.01 | activator protein 1 | 2104-2114 | (−) | 1.000 | 0.979 | ggTGACtccat | 218 |
| VMYB/VMYB.02 | v-Myb | 2113-2121 | (+) | 1.000 | 0.912 | ccaAACGgg | 219 |
| ETSF/ELK1.01 | Elk-1 | 2114-2129 | (+) | 0.866 | 0.83 | caaacgGGATgatcca | 220 |
| NFKB/NFKAPPAB.02 | NF-kappaB | 2118-2129 | (+) | 0.929 | 0.815 | cGGGATgatcca | 221 |
| AREB/AREB6.04 | AREB6 (Atplal Regulatory | 2134-2142 | (−) | 1 | 0.997 | ctGTTTctt | 222 |
| | element binding factor 6) | | | | | | |
| ZFI1A/ZID.01 | zinc finger with inter- | 2146-2158 | (+) | 1 | 0.889 | cgGCTCtaacaca | 223 |
| | action domain | | | | | | |
| XBBF/RFX1.02 | X-box binding protein REX1 | 2149-2166 | (+) | 1 | 0.899 | ctctaacacaaGCAAcag | 224 |
| CMYB/CMYB.01 | c-Myb, important in hema- | 2157-2174 | (−) | 1 | 0.916 | gtttgttgctGTTGcttg | 225 |
| | topoesis, cellular | | | | | | |
| | equivalent to avian myo- | | | | | | |
| | blastosis virus oncogene | | | | | | |
| | v.-myb | | | | | | |
| CREB/TAXCREB.02 | Tax/CREB complex | 2205-2219 | (−) | 0.750 | 0.741 | gaggaaaTACGtctt | 226 |
| ETSF/ETS2.01 | c-Ets-2 binding site | 2208-2121 | (−) | 1.000 | 0.907 | aagaGGAAatacgt | 227 |
| NFAT/NFAT.01 | Nuclear factor of | 2210-2221 | (−) | 1.000 | 0.962 | aagagGAAAtac | 228 |
| | activated T-cells | | | | | | |
| EVI1/EVI1.02 | ectopic viral integration | 2222-2232 | (−) | 1.000 | 0/854 | tgagAAGAtta | 229 |
| | site 1 encoded factor | | | | | | |
| OAZF/ROAZ.01 | Rat C2H2 Zn finger protein | 2231-2246 | (+) | 0.750 | 0.789 | cagCATCcttggtga | 230 |
| | involved in olfactory | | | | | | |
| | neuronal differentiation | | | | | | |
| EBOR/DELTAEF1.01 | deltaEF1 | 2238-2248 | (−) | 1.000 | 0.985 | cctcACCTaag | 231 |
| CREB/CREBP1.01 | cAMP-responsive element | 2239-2246 | (−) | 0.766 | 0.801 | tcACCTaa | 232 |
| | binding protein 1 | | | | | | |
| HNF4/HNF4.02 | Hepatic nuclear factor 4 | 2253-2267 | (+) | 0.750 | 0.776 | tgggtccAGAGgcct | 233 |
| GATA/GATA.01 | GATA binding site | 2262-2272 | (−) | 1.000 | 1.000 | aGATAAggcct | 234 |
| | (consensus) | | | | | | |
| CREB/E4BP4.01 | E4BP4, bZIP domain, | 2265-2276 | (+) | 0.758 | 0.840 | ccttatCTAAaa | 235 |
| | transcriptional repressor | | | | | | |
| TBPF/ATATA.01 | Avian C-type LTR TATA box | 2265-2274 | (−) | 0.834 | 0.850 | tTAGATAagg | 236 |
| XBBF/MIF1.01 | MIBP-1/RFX1 complex | 2281-2298 | (−) | 0.800 | 0.774 | acggtgcccaGCCAccca | 237 |
| EBOX/USF.02 | upstream stimulating | 2304-2311 | (+) | 0.875 | 0.931 | aCACATgt | 238 |
| | factor | | | | | | |
| VBPF/VBP.01 | PAR-type chicken | 2305-2314 | (−) | 1.000 | 0.863 | aTTACatgtg | 239 |
| | vitellogenin promoter- | | | | | | |
| | binding protein | | | | | | |
| IKRS/IK2.01 | Ikaros 2, potential | 2310-2321 | (−) | 1.000 | 0.960 | tgctGGGAttac | 240 |
| | regulator of lymphocyte | | | | | | |
| | differentiation | | | | | | |
| NRSF/NRSF.01 | neuron-restrictive | 2315-2335 | (+) | 1.000 | 0.685 | cccAGCActttggaaggccga | 241 |
| | silencer factor | | | | | | |
| TANT/TANTIGEN.01 | Major T-antigen binding | 2326-2344 | (+) | 0.759 | 0.872 | ggaaggcCGAGgcaggtgg | 242 |
| | site | | | | | | |
| AREB/AREB6.01 | AREB6 (Atplal regulatory | 2335-2347 | (−) | 1.000 | 0.921 | gtccACCTgcct | 243 |
| | element_binding factor 6) | | | | | | |
| MYOD/MYOD.02 | myoblast determining | 2336-2345 | (−) | 1.000 | 0.992 | tcCACCtgcc | 244 |
| | factor | | | | | | |
| EBOX/SREBP1.02 | sterol regulatory element- | 2344-2354 | (+) | 1.000 | 0.791 | gaTCACccgag | 245 |
| | binding protein 1 | | | | | | |
| RARF/RAR.01 | Retinoie acid receptor, | 2353-2362 | (+) | 0.897 | 0.961 | aggTCAGgag | 246 |
| | member of nuclear | | | | | | |
| | receptors | | | | | | |
| CREB/HLF.01 | hepatic leukemia factor | 2384-2393 | (−) | 0.770 | 0.857 | GTTTcgccat | 247 |
| CLOX/CDPCR3HD.01 | cut-like homeodomain | 2394-2403 | (−) | 0.929 | 0,941 | tattGATGag | 248 |
| | protein | | | | | | |
| OCT1/OCT1.02 | octamer-binding factor | 2409-2418 | (+) | 1.000 | 0.941 | aATGCaaaaa | 249 |
| MYT1/MYT1.01 | MyT1 zinc finger tran- | 2414-2425 | (+) | 0.750 | 0.775 | aaAAATtagctt | 250 |
| | scription factor involved | | | | | | |
| | in primary neurogenesis | | | | | | |
| HAML/AML1.01 | runt-factor AML-1 | 2428-2433 | (+) | 1.000 | 1.000 | tgTGGT | 251 |
| IKRS/IK2.01 | Ikaros 2, potential | 2445-2456 | (−) | 1.000 | 0.967 | ggctGGGAttac | 252 |
| | regulator of lymphocyte | | | | | | |
| | differentiation | | | | | | |
| AHRR/AHRARNT.02 | aryl hydrocarbon/Arnt | 24875-2493 | (−) | 0.750 | 0.772 | tgggtttGAGTgttctcc | 253 |
| | heterodimers, fixed core | | | | | | |
| CHOP/CHOP.01 | heterodimers of CHOP and | 2500-2512 | (−) | 1.000 | 0.943 | cacTGCAatctcc | 254 |
| | C/EBPalpha | | | | | | |
| OCT1/OCT1.01 | octamer-binding factor 1 | 2517-2535 | (+) | 1.000 | 0.802 | gagatTATGccactgcact | 255 |
| MEF2/MEF2.01 | myogenic enhancer factor 2 | 2565-2580 | (+) | 0.750 | 0.752 | ctcAAAAaataaaata | 256 |
| CDXF/CDX2.01 | Cdx-2 mammalian caudal | 2571-2589 | (−) | 1.000 | 0.835 | caaaggtTTTAttttattt | 257 |
| | related intestinal | | | | | | |
| | transcr. Factor | | | | | | |
| EVI1/EVI1.03 | ectopic viral integration | 2571-2581 | (+) | 0.750 | 0.788 | aaataAAATaa | 258 |
| | site 1 encoded factor | | | | | | |
| RPOA/POLYA.01 | Mammalian C-Type LTR Poly | 2576-2593 | (+) | 1.000 | 0.806 | aAATAAAacctttggggc | 259 |
| | A signal | | | | | | |
| E2FF/E2F.02 | E2F, involved in cell | 2586-2593 | (−) | 1.000 | 0.849 | gcccCAAA | 260 |
| | cycle regulation, | | | | | | |
| | interacts with Rb p107 | | | | | | |
| | protein | | | | | | |
| XSEC/STAF.01 | Se-Cys tRNA gene tran- | 2606-2627 | (−) | 1.000 | 0.812 | aatcCCCAgaattctggactct | 261 |
| | scription activating | | | | | | |
| | factor | | | | | | |
| NFKB/NFKAPPAB.02 | NF-kappaB | 2621-2632 | (+) | 0.929 | 0.877 | gGGGATtttcaa | 262 |
| HNF1/HNF1.02 | Hepatic nuclear factor 1 | 2635-265 | (+) | 0.859 | 0.778 | gGCTAttcaataaatgg | 263 |
| RPOA/LPOLYA.01 | Lentiviral Poly A signal | 2642-2649 | (+) | 1.000 | 0.971 | cAATAAAt | 264 |
| TBPF/TATA.01 | cellular and viral TATA | 2646-2660 | (−) | 1.000 | 0.925 | ataTAAAtcccattt | 265 |
| | box elements | | | | | | |
| HMTB/MTBF.01 | muscle-specific Mt binding | 2649-2657 | (+) | 1.000 | 0.901 | tgggATTTa | 266 |
| | site | | | | | | |
| CREB/HLF.01 | hepatic leukemia factor | 2659-2668 | (−) | 1.000 | 0.869 | GTTAtgtgat | 267 |
| VBPF/VBP.01 | PAR-type chicken | 2659-2668 | (−) | 0.830 | 0.886 | gTTATgtgat | 268 |
| | vitellogenin promoter- | | | | | | |
| | binding protein | | | | | | |
| CREB/CREB.03 | cAMP-responsive element | 2681-2692 | (+) | 1.000 | 0.915 | tcTGACgcagtt | 260 |
| | binding protein | | | | | | |
| GATA/GATA1.01 | GATA binding factor 1 | 2692-2705 | (−) | 1.000 | 0.963 | tagttGATAggaga | 270 |
| CLOX/CLOX.01 | Clox | 2700-2714 | (−) | 1.000 | 0.823 | aaaATCGaatagttg | 271 |
| NFAT/NFAT.01 | Nuclear factor of | 2709-2720 | (−) | 1.000 | 0.972 | tgaagGAAAatc | 272 |
| | activated T-cells | | | | | | |
| GFI1/GFI1.01 | growth factor independence | 2728-2751 | (+) | 1.000 | 0.943 | aatttaaaAATCacatcaagggat | 273 |
| | 1 zinc finger protein acts | | | | | | |
| | as transcriptional | | | | | | |
| | repressor | | | | | | |
| MEF2/MEF2.05 | MEF2 | 2728-2737 | (+) | 1.000 | 0.969 | aattTAAAaa | 274 |
| GATA/GATA3.02 | GATA-binding factor 3 | 2746-2755 | (+) | 0.812 | 0.904 | agGGATctaa | 275 |
| FKHD/FREAC3.01 | Fork head Related | 2747-2762 | (+) | 0.750 | 0.849 | gggatCTAAataaaga | 276 |
| | Activator-3 | | | | | | |
| MEF2/MEF2.05 | MEF2 | 2749-2758 | (+) | 1.000 | 0.960 | gatcTAAAta | 277 |
| RPOA/LPOLYA.01 | Lentiviral Poly A signal | 2754-2761 | (+) | 1.000 | 0.992 | aAATAAAg | 278 |
| HMTB/MTBF.01 | muscle-specific Mt binding | 2766-2774 | (−) | 1.000 | 0.911 | agctATTTa | 279 |
| | site | | | | | | |
| VMYB/VMYB.02 | v-Myb | 2780-2788 | (−) | 0.819 | 0.892 | cccAACTga | 280 |
| SMAD/SMAD3.01 | Smad3 transcription factor | 2788-2795 | (+) | 1.000 | 0.993 | GTCTggtc | 281 |
| | involved in TGF beta | | | | | | |
| | signaling | | | | | | |
| HNF4/HNF4.02 | Hepatic nuclear factor 4 | 2801-2815 | (−) | 0.750 | 0.778 | aaggaccAAACctct | 282 |
| MYT1/MYT1.02 | MyT1 zinc finger tran- | 2815-2825 | (−) | 1.000 | 0.897 | agaAAGTtcta | 283 |
| | scription factor involved | | | | | | |
| | in primary neurogenesis | | | | | | |
| HEAT/HSF1.01 | heat shock factor 1 | 2816-2825 | (−) | 1.000 | 0.98 | AGAAagttct | 284 |
| MZF1/MZF1.01 | MZF1 | 2847-2854 | (−) | 1.000 | 0.978 | aatGGGGa | 285 |
| TBPF/TATA.02 | Mammalian C-Type LTR TATA | 2852-2861 | (−) | 0.885 | 0.914 | tcTGTAAAAT | 286 |
| | box | | | | | | |
| GATA/GATA1.03 | GATA-binding factor 1 | 2856-2868 | (+) | 1.000 | 0.981 | tacaGATAaaggg | 287 |
| ETSF/PU1.01 | Pu. 1 (Pul20) Ets-like | 2868-2883 | (+) | 1.000 | 0.870 | gaatgaGGAAgggtaa | 288 |
| | transcription factor | | | | | | |
| | identified in lymphoid B | | | | | | |
| | cells | | | | | | |
| CREB/HLF.01 | hepatic leukemia factor | 2885-2894 | (−) | 1.000 | 0.892 | GTTActtcat | 289 |
| VBPF/VBP.01 | PAR-type chicken | 2885-2894 | (−) | 1.000 | 0.913 | gTTACttcat | 290 |
| | vitellogenin promoter- | | | | | | |
| | binding protein | | | | | | |
| RORA/RORA2.01 | RAR-related orphan | 2890-2902 | (+) | 1.000 | 0.928 | gtaacttGGTCaa | 291 |
| | receptor alpha 2 | | | | | | |
| LDPS/LDSPOLYA.01 | Lentiviral Poly A down- | 2932-2947 | (+) | 1.000 | 0.900 | ggaGTGTgtgtgcatg | 292 |
| | stream element | | | | | | |
| EBOX/USF.02 | upstream stimulating | 2943-2950 | (−) | 0.875 | 0.933 | aCACATgc | 293 |
| | factor | | | | | | |
| NFKB/NFKAPPAB.01 | NF-kappaB (p50) | 2966-2975 | (−) | 1.000 | 0.885 | GGGGgtgccc | 294 |
| MINI/MUSCLE_INI.03 | Muscle Initiator Sequence | 2967-2987 | (+) | 1.000 | 0.879 | ggcacccccCACCccgacccc | 295 |
| REBV/EBVR.01 | Epstein-Barr virus tran- | 2967-2987 | (−) | 1.000 | 0.828 | ggggtcggggtggggGGTGcc | 296 |
| | scription factor R | | | | | | |
| EGRF/WT1.01 | Wilms Tumor Suppressor | 2968-2980 | (−) | 1.000 | 0.909 | gggTGGGgggtgc | 297 |
| SP1F/GC.01 | GC box elements | 2970-2983 | (−) | 0.872 | 0.897 | tcggGGTGgggggt | 298 |
| RREB/RREB1.01 | Ras-responsive element | 2973-2986 | (+) | 1.000 | 0.826 | cCCCAccccgaccc | 299 |
| | binding protein 1 | | | | | | |
| PCAT/ACAAT.01 | Avian C-type LTR CCAAT box | 2986-2994 | (+) | 0.793 | 0.866 | ccACCACtg | 300 |
| ARP1/ARP1.01 | apolipoprotein AI | 2993-3008 | (−) | 1.000 | 0.861 | tgattcCTTGctctca | 301 |
| | regulatory protein 1 | | | | | | |
| MYT1/MYT1.02 | MyT1 zinc finger tran- | 3015-3025 | (−) | 1.000 | 0.893 | tcaAAGTtgtt | 302 |
| | scription factor involved | | | | | | |
| | in primary neurogenesis | | | | | | |
| IRFF/ISRE.01 | interferon-stimulated | 3033-3047 | (+) | 1.000 | 0.800 | ctgtaccaGAAActc | 303 |
| | response element | | | | | | |
| EGRF/WT1.01 | Wilms Tumor Suppressor | 3053-3065 | (−) | 1.000 | 0.900 | gtgTGGGaggctc | 304 |
| RARF/RAR.01 | Retinoic acid receptor, | 3085-3094 | (−) | 1.000 | 0.987 | aggTCACcca | 305 |
| | member of nuclear | | | | | | |
| | receptors | | | | | | |
| RORA/RORA1.01 | RAR-related orphan | 3088-3100 | (−) | 1.000 | 0.956 | agaagaaGGTCac | 306 |
| | receptor alpha 1 ectopic | | | | | | |
| | viral integration site 1 | | | | | | |
| EVI1/EVI1.01 | encoded factor | 3092-3107 | (−) | 1.000 | 0.728 | agccAAGAgaagaagg | 307 |
| OCT1/OCT1.05 | octamer-binding factor 1 | 3124-3137 | (+) | 0.888 | 0.911 | ctcattttaATTCa | 308 |
| OCTB/TST1.01 | POU-factor Tst-1/Oct-6 | 3125-3139 | (−) | 1.000 | 0.961 | agtgAATTaaaatga | 309 |
| RBIT/BRIGHT.01 | Bright, B B326 cell | 3127-3139 | (−) | 1.000 | 0.959 | agtgaATTAaaat | 310 |
| | regulator of IgH tran- | | | | | | |
| | scription | | | | | | |
| NKXH/NKX25.02 | homeo domain factor | 3129-3136 | (+) | 1.000 | 0.874 | tTTAAttc | 311 |
| | Nkx-2.5/Csx, tinman | | | | | | |
| | homolog low affinity sites | | | | | | |
| GREF/PRE.01 | Progesterone receptor | 3140-3166 | (+) | 1.000 | 0.847 | ttcatagtgttgtttTGTTctcgtttt | 312 |
| | binding site | | | | | | |
| RPOA/POLYA.01 | Mammalian C-type LTR Poly | 3142-3159 | (−) | 0.822 | 0.711 | gAACAAAacaacactatg | 313 |
| | A signal | | | | | | |
| AHRR/AHR.01 | aryl hydrocarbon/dioxin | 3193-3210 | (−) | 0.750 | 0.840 | actccagcttGGGTgaga | 314 |
| | receptor | | | | | | |
| GFI1/GFI1.01 | growthfactor independence | 3213-3236 | (+) | 1.000 | 0.953 | agtgctgcAATCacagctcattgc | 315 |
| | 1 zinc finger protein acts | | | | | | |
| | as transcriptional | | | | | | |
| | repressor | | | | | | |
| LYMF/LYF1.01 | LyF-1, enriched in B and T | 3277-3285 | (−) | 1.000 | 0.988 | tttGGGAgg | 316 |
| | lymphocytes | | | | | | |
| HOBO/HOGNESS.01 | Imperfect Hogness/Goldberg | 3284-3315 | (−) | 0.764 | 0.917 | cacggtggctcacaccTGTAatcccagcactt | 317 |
| | Box | | | | | | |
| IKRS/1K2.01 | Ikaros 2, potential | 3287-3298 | (+) | 1.000 | 0.960 | tgctGGGAttac | 318 |
| | regulator of lymphocyte | | | | | | |
| | differentiation | | | | | | |
| MYOD/E47.02 | TAL1/E47 dimers | 3293-3308 | (+) | 1.000 | 0.932 | gattaCAGGtgtgagc | 319 |
| AREB/AREB6.02 | AREB6 (Atpla1 regulatory | 3295-3306 | (−) | 1.000 | 0.979 | tcaCACCtgtaa | 320 |
| | element binding factor 6) | | | | | | |
| BRAC/TBX5.01 | T-Box factor 5 site | 3297-3308 | (+) | 1.000 | 0.991 | acaGGTGtgagc | 331 |
| | (TBX5), mutations related | | | | | | |
| | to Holt-Oram syndrome | | | | | | |
| TBPF/MTATA.01 | Muscle TATA box | 3323-3339 | (−) | 1.000 | 0.888 | ctgttTAAAaccctata | 322 |
| FKHD/FREAC2.01 | Fork head Related | 3327-3342 | (+) | 1.000 | 0.854 | gggtttTAAAcagtaa | 323 |
| | Activator-2 | | | | | | |
| MEF2/MEF2.05 | MEF2 | 3329-3338 | (+) | 1.000 | 0.986 | gtttTAAAca | 324 |
| CEBP/CEBP.02 | C/EBP binding site | 3359-3376 | (−) | 0.957 | 0.857 | tgcctgcgGTAAGtcgta | 325 |
| NOLF/OLF1.01 | olfactory neuron-specific | 3383-3404 | (−) | 1.000 | 0.822 | aaagggTCCCcccggggcctgt | 326 |
| | factor | | | | | | |
| AP2F/AP2.01 | activator protein 2 | 3388-3399 | (−) | 0.976 | 0.895 | gtCCCCccgggg | 327 |
| MZFl/MZF1.01 | MZF1 | 3391-3398 | (+) | 1.000 | 0.980 | cggGGGGa | 328 |
| HEN1/HEN1.01 | HEN1 | 3415-3436 | (+) | 1.000 | 0.873 | ccagggtaCAGCtgtgacaccg | 329 |
| AP4R/AP4.01 | activator protein 4 | 3421-3430 | (−) | 1.000 | 0.974 | caCAGCtgta | 330 |
| GATA/GATA1.02 | GATA-binding factor 1 | 3448-3461 | (−) | 1.000 | 0.934 | actggGATAatcca | 331 |
| NFKB/NFKAPPAB.02 | NF-kappaB | 3448-3459 | (−) | 0.929 | 0.822 | tGGGATaatcca | 1332 |
| FKHD/HFH8.01 | HNF-3/Fkh Homolog-8 | 3461-3473 | (+) | 1.000 | 0.970 | tagatAAACaaaa | 333 |
| GATA/GATA.01 | GATA binding site | 3462-3472 | (+) | 1.000 | 0.949 | aGTAAAacaaa | 334 |
| | (consensus) | | | | | | |
| SORY/SRY.01 | sex-determining region Y | 3464-3475 | (+) | 1.000 | 0.946 | ataaACAAaaat | 335 |
| | gene product | | | | | | |
| CREB/CREB.02 | cAMP-responsive element | 3480-3491 | (−) | 1.000 | 0.87 | ggaaTGACgatc | 336 |
| | binding protein | | | | | | |
| PAX3/PAX3.01 | Pax-3 paired domain | 3482-3494 | (+) | 1.000 | 0.785 | TCGTcattccatt | 337 |
| | protein, exressed in | | | | | | |
| | embryogenesis, mutations | | | | | | |
| | correlate to Waardenburg | | | | | | |
| | Syndrome | | | | | | |
| TEAF/TEF1.01 | TEF-1 related muscle | 3484-3495 | (+) | 1.000 | 0.834 | gtCATTccattt | 338 |
| | factor | | | | | | |
| PAX1/PAX1.01 | Pax1 paired domain | 3490-3507 | (+) | 0.750 | 0.733 | CCATttctctctgtatat | 339 |
| | protein, expressed in the | | | | | | |
| | developing vertebral | | | | | | |
| | column of mouse embryos | | | | | | |
| NFAT/NFAT.01 | Nuclear factor of | 3508-3519 | (−) | 1.000 | 0.966 | gcttgGAAAaat | 340 |
| | activated T-cells | | | | | | |
| BARB/BARBIE.01 | barbiturate-inducible | 3514-3528 | (−) | 1.000 | 0.885 | atgaAAAGggcttgg | 341 |
| | element | | | | | | |
| OCT1/OCT1.02 | octamer-binding factor 1 | 3520-3529 | (−) | 0.763 | 0.823 | cATGAaaagg | 342 |
| AP1F/TCF11MAFG.01 | TCF11/MafG heterodimers, | 3522-3543 | (+) | 0.777 | 0.808 | ttttcaTGAAtgatcagttatt | 343 |
| | binding to subclass of AP1 | | | | | | |
| | sites | | | | | | |
| PITI1/PIT1.01 | Pit1, GHF-1 pituitary | 3527-3536 | (−) | 1.000 | 0.855 | gatcATTCat | 344 |
| | specific pou domain | | | | | | |
| | transcription factor | | | | | | |
| VMYB/VMYB.01 | v-Myb | 3534-3543 | (−) | 0.876 | 0.938 | aatAACTgat | 345 |
| ETSF/ETS2.01 | c-Ets-2 binding site | 3537-3550 | (−) | 1.000 | 0.946 | tgcaGGAAataact | 346 |
| GFI1/GFI1.01 | growth factor independence | 3541-3564 | (−) | 1.000 | 0.977 | aaaaaaaaAATCagtgcaggaaat | 347 |
| | 1 zinc finger protein acts | | | | | | |
| | as transcriptional | | | | | | |
| | repressor | | | | | | |
| AP1F/AP1F1.01 | activator protein 1 | 3592-3602 | (−) | 1.000 | 0.968 | ggTGACagagt | 348 |
| EBOX/SREBP1.02 | sterol regulatory element- | 3617-3627 | (−) | 0.750 | 0.791 | gaTCATgccac | 349 |
| | binding protein 1 | | | | | | |
| PAX3/PAX3.01 | Pax-3 paired domain | 3628-3640 | (+) | 0.780 | 0.765 | TCGGctcgctgca | 350 |
| | protein, expressed in | | | | | | |
| | embryogenesis, mutations | | | | | | |
| | correlate to Waardenburg | | | | | | |
| | Syndrome | | | | | | |
| HEAT/HSF1.01 | heat shock factor 1 | 3663-3672 | (−) | 1.000 | 0.937 | AGAAgaatcg | 351 |
| XSEC/STAF.02 | Se-Gys tRNA gene tran- | 3706-3726 | (+) | 0.810 | 0.870 | gagtACCAtcatgcccggcta | 352 |
| | scription activating | | | | | | |
| | factor | | | | | | |
| P53F/P53.01 | tumor suppressor p53 | 3712-3731 | (+) | 1.000 | 0.660 | catCATGcccggctaatttt | 353 |
| MEF2/RSRFC4.02 | related to serum response | 3729-3745 | (−) | 1.000 | 0.885 | ctactaaaAATAcaaaa | 354 |
| | factor, C4 | | | | | | |
| SRFF/SRF.01 | serum response factor | 3755-3772 | (+) | 0.773 | 0.653 | ttcaccaTATTggccagg | 355 |
| ECAT/NFY.02 | nuclear factor Y | 3760-3770 | (−) | 1.000 | 0.920 | tggCCAAtatg | 356 |
| | (Y box binding factor) | | | | | | |
| HNF4/HNF4.02 | Hepatic nuclear factor 4 | 3788-3802 | (−) | 0.750 | 0.784 | cagatcgCAAGgtcc | 357 |
| LYMF/LYP1.01 | LyF-1, enriched in B and T | 3813-3821 | (−) | 1.000 | 0.988 | tttGGGAgg | 358 |
| | lymphocytes | | | | | | |
| HOBO/HOGNESS.01 | Imperfect Hogness/Godberg | 3820-3851 | (−) | 0.764 | 0.928 | | |
| cgcggtggctcacgccTGTAatcccagcactt | 359 | | | | | | |
| | Box | | | | | | |
| IKRS/1K2.01 | Ikaros 2, potential | 3823-3834 | (+) | 1.000 | 0.960 | tgctGGGAttac | 360 |
| | regulator of lymphocyte | | | | | | |
| | differentiation | | | | | | |
| CREB/TAXCREB.01 | Tax/CREB complex | 3834-3848 | (−) | 0.784 | 0.806 | ggtggctCACgcctg | 361 |
| EBOX/MYCMAX.03 | MYC-MAX binding sites | 3848-3857 | (−) | 0.813 | 0.920 | gcCAGGcgcg | 362 |
| GATA/GATA3.02 | GATA-binding factor 3 | 3866-3875 | (+) | 0.875 | 0.910 | acTGATataa | 363 |
| EVI1/EVI1.04 | ectopic viral integration | 3868-3882 | (+) | 1.000 | 0.809 | tGATAtaaaaagaat | 364 |
| | site 1 encoded factor | | | | | | |
| MEF2/MEF2.05 | MEF2 | 3869-3878 | (+) | 1.000 | 0.968 | gataTAAAaa | 365 |
| TBPF/TATA.01 | cellular and viral TATA | 3870-3884 | (+) | 1.000 | 0.958 | ataTAAAaagaattt | 366 |
| | box elements | | | | | | |
| RPOA/APOLYA.01 | Avian C-type LTR Poly A | 3874-3888 | (−) | 0.829 | 0.754 | AAAAAAattcttttt | 367 |
| | signal | | | | | | |
| MEF2/MEF2.05 | MEF2 | 3884-3893 | (−) | 1.000 | 0.969 | aattTAAAaa | 368 |
| EBOX/SREBP1.02 | sterol regulatory element- | 3899-3909 | (+) | 0.750 | 0.849 | ttTCTCcccac | 369 |
| | binding protein 1 | | | | | | |
| MZF1/MZF1.01 | MZF1 | 3903-3910 | (−) | 1.000 | 1.000 | agtGGGGa | 370 |
| MINI/MUSCLE_INI.03 | Muscle Initiator Sequence | 3904-3924 | (+) | 1.000 | 0.881 | ccccactccCACCcccaggct | 371 |
| RREB/RREB1.01 | Ras-responsive element | 3904-3917 | (+) | 1.000 | 0.831 | cCCCActcccaccc | 372 |
| | binding protein 1 | | | | | | |
| EGRF/WT1.01 | Wilms Tumor Suppressor | 3905-3917 | (−) | 1.000 | 0.941 | gggTGGGagtggg | 373 |
| AP2F/AP2.01 | activator protein 2 | 3913-3924 | (+) | 0.976 | 0.929 | caCCCCcaggct | 374 |
| TBPF/MTATA.01 | Muscle TATA box | 3919-3945 | (+) | 1.000 | 0.917 | ccttaTAAAgcagcctc | 375 |
| HAML/AMLI.01 | Runt-factor AML-1 | 3968-3973 | (+) | 1.000 | 1.000 | tgTGGT | 376 |
| ETSF/ELK1.02 | Elk-1 | 3983-3996 | (+) | 1.000 | 0.926 | gggcccGGAAttgg | 377 |
| LYMF/THIE47.01 | Thing 1/E47 heterodinner, | 3991-4006 | (+) | 1.000 | 0.910 | aattgggtCTGGggca | 378 |
| | TH 1 bHLH member specific | | | | | | |
| | expression in a variety of | | | | | | |
| | embryonic tissues | | | | | | |
| PAX5/PAX5.01 | B-cell-specific activating | 4016-4043 | (−) | 0.904 | 0.862 | cccaagAGCAgggcagagaagcaagcaa | 379 |
| | protein | | | | | | |
| LTUP/TAACC.01 | Lentiviral TATA upstream | 4037-4059 | (−) | 1.000 | 0.838 | tgcccctgaggCTAACCccaaga | 380 |
| | element | | | | | | |
| PAX5/PAX5.01 | B-cell-specific activating | 4050-4077 | (+) | 0.952 | 0.820 | ctcaggGGCAgggttgagagtcaggctt | 381 |
| | protein | | | | | | |
| PCAT/CLTR_CAAT.01 | Mammalian C-type LTR CCAAT | 4056-4080 | (−) | 0.803 | 0.758 | gcCAAGcctgactctcaaccctgcc | 382 |
| | box | | | | | | |
| MYOD/MYF5.01 | Myf5 myogenic bHLH protein | 4082-4093 | (+) | 1.000 | 0.920 | aggCAGCaggag | 383 |
| ETSF/ELK1.01 | Elk-1 | 4084-4099 | (+) | 0.800 | 0.832 | gcagcaGGAGgtccag | 384 |
| SMAD/SMAD3.01 | Smad3 transcription factor | 4094-4101 | (−) | 1.000 | 0.996 | GTCTggac | 385 |
| | involved in TOF-beta | | | | | | |
| | signaling | | | | | | |
| GATA/GATA2.02 | GATA-binding factor 2 | 4120-4129 | (+) | 1.000 | 0.917 | ggaGATAcca | 386 |
| HMTB/MTBF.01 | Muscle-specific Mt binding | 4121-4129 | (−) | 0.884 | 0.912 | tggtATCTc | 387 |
| | site | | | | | | |
| EGRF/WT1.01 | Wilms Tumor Suppressor | 4131-4143 | (+) | 0.813 | 0.893 | gagAGGGcgcatc | 388 |
| PERO/PPARA.01 | PPAR/RXR heterodimers | 4143-4162 | (−) | 1.000 | 0.694 | ctgaaacaggaaAAAGgcag | 389 |
| GKLF/GKLF.01 | gut-enriched Krueppel-like | 4146-4159 | (−) | 0.936 | 0.918 | aaacaggaaaAAGG | 390 |
| | factor | | | | | | |
| NFAT/NFAT.01 | Nuclear factor of | 4147-4158 | (−) | 1.000 | 0.984 | aacagGAAAaag | 391 |
| | activated T-cells | | | | | | |
| AREB/AREB6.04 | AREB6 (Atpl al regulatory | 4154-4162 | (+) | 1.000 | 1.000 | ctGTTTcag | 392 |
| | element binding factor 6) | | | | | | |
| SORY/SRY.01 | sex-determining region Y | 4181-4192 | (−) | 1.000 | 0.950 | aaaaACAAaaca | 393 |
| | gene product | | | | | | |
| FKHD/HFH2.01 | HNF-3/Fkh Homolog 2 | 4183-4194 | (−) | 1.000 | 0.938 | aaaaaAACAaaa | 394 |
| EGRF/WT1.01 | Wilms Tumor Suppressor | 4210-4222 | (−) | 0.813 | 0.871 | gagAGGGagggag | 395 |
| EGRF/WT1.01 | Wilms Tumor Suppressor | 4222-4234 | (−) | 0.813 | 0.871 | gagAGGGagggag | 396 |
| GKLF/GKLF.01 | gut-enriched Krueppel-like | 4252-4265 | (−) | 1.000 | 0.916 | agagagagagAGGG | 397 |
| | factor | | | | | | |
| SP1F/SP1.01 | stimulating protein 1 SP1, | 4267-4279 | (−) | 0.844 | 0.888 | ggagGGAGgggga | 398 |
| | ubiquitous zinc finger | | | | | | |
| | transcription factor | | | | | | |
| GKLF/GKLF.01 | gut-enriched Krueppel-like | 4269-4282 | (−) | 0.950 | 0.936 | gaaggagggaGGGG | 399 |
| | factor | | | | | | |
| OCT1/OCT1.02 | octamer-binding factor 1 | 4321-4330 | (+) | 1.000 | 0.849 | gATGCacata | 400 |
| EVI1/EVI1.06 | ectopic viral integration | 4346-4354 | (−) | 0.750 | 0.835 | acaAGGTag | 401 |
| | site 1 encoded factor | | | | | | |
| TCFF/TCF11.01 | TCFl1/KCR-Fl/Nrfl | 4353-4365 | (+) | 1.000 | 0.991 | GTCAtcctgctgt | 402 |
| | homodimers | | | | | | |
| MINI/MUSCLE_INI.01 | Muscle Initiator Sequence | 4383-4403 | (+) | 1.000 | 0.857 | tccctcctCCACaccagcaga | 403 |
| NRSF/NRSF.01 | neuron-restrictive | 4412-4432 | (+) | 1.000 | 0.746 | ttcAGCAacaagaatagccga | 404 |
| | silencer factor | | | | | | |
| CLOX/CDPCR3.01 | cut-like homeodomain | 4414-4428 | (+) | 0.888 | 0.770 | cagcaacaagaATAG | 405 |
| | protein | | | | | | |
| PCAT/CLTR_CAAT.01 | Mammalian C-type LTR CCAAT | 4455-4479 | (+) | 0.803 | 0.761 | ccCAAGaagcatcctgcaggctttc | 406 |
| | box | | | | | | |
| BARB/BARBIE.01 | barbiturate-inducible | 4475-4489 | (−) | 1.000 | 0.875 | tcaaAAAGcagaaag | 407 |
| | element | | | | | | |
| MEF2/MMEF2.01 | myocyte enhancer factor | 4489-4504 | (−) | 1.000 | 0.892 | tgcttTAAAatacact | 408 |
| TBPF/TATA.02 | Mammalian C-type LTR TATA | 4494-4503 | (−) | 0.927 | 0.938 | gcTTTAAAAt | 409 |
| | box | | | | | | |
| TBPF/ATATA.01 | Avian C-type LTR TATA box | 4520-4529 | (+) | 0.896 | 0.809 | cTATGTAtgc | 410 |
| MYT1/MYT1.01 | MyT1 zinc finger tran- | 4531-4542 | (−) | 0.750 | 0.776 | caTAGTtaactg | 411 |
| | scription factor involved | | | | | | |
| | in primary neurogenesis | | | | | | |
| GATA/GATA3.02 | GATA-binding factor 3 | 4544-4553 | (+) | 1.000 | 0.904 | ctAGATgtta | 412 |
| FKHD/XFD3.01 | Xenopus fork head domain | 4545-4558 | (−) | 1.000 | 0.836 | aaggttAACAtcta | 413 |
| | factor | | | | | | |
| MYT1/MYT1.01 | MyT1 zinc finger tran- | 4548-4559 | (−) | 0.750 | 0.775 | aaAGGTtaacat | 414 |
| | scription factor involved | | | | | | |
| | in primary neurogenesis | | | | | | |
| AP4R/TALIBETA-E47.01 | Tal-1 beta/E47 heterodimer | 4567-4582 | (+) | 1.000 | 0.884 | aaacaCAGAtggaggc | 415 |
| EGRF/EGR1.01 | Egr-1/Krox-24/NGFI-A | 4614-4625 | (+) | 1.000 | 0.780 | ttctgtgGGCGg | 416 |
| | immediate-early gene | | | | | | |
| | product | | | | | | |
| ZFIA/ZID.01 | zinc finger with inter- | 4639-4651 | (+) | 1.000 | 0.918 | cgGCTCcagcctc | 417 |
| | action domain | | | | | | |
| CREB/TAXCREB.02 | Tax/CREB complex | 4657-4671 | (+) | 1.000 | 0.700 | cgggatcTGCGggaa | 418 |
| CEBP/CEBP.02 | C/EBP binding site | 4660-4677 | (+) | 0.858 | 0.875 | gatctgcgGGAAGacacg | 419 |
| E2FF/E2F.01 | E2F, involved in cell | 4662-4676 | (+) | 0.750 | 0.762 | tctgcggGAAGacac | 420 |
| | cycle regulation, | | | | | | |
| | interacts with Rb p107 | | | | | | |
| | protein | | | | | | |
| EBOX/NMYC.01 | N-Myc | 4671-4682 | (−) | 1.000 | 0.901 | ttcccCGTGtct | 421 |
| CLOX/CDP.01 | cut-like homeodomain | 4703-4714 | (−) | 0.757 | 0.751 | tcATTAatcaaa | 422 |
| | protein | | | | | | |
| HNF1/HNF1.01 | hepatic nuclear factor 1 | 4706-4720 | (+) | 0.775 | 0.836 | gATTAatgatttatt | 423 |
| CART/CART1.01 | Cart-1 (cartilage homeo- | 4713-4730 | (+) | 0.791 | 0.881 | gatTTATtttgattaacg | 424 |
| | protein 1) | | | | | | |
| RPOA/LPOLYA.01 | Lentiviral Poly A signal | 4714-4721 | (−) | 1.000 | 0.963 | aAATAAAt | 425 |
| HNF1/TTNF1.01 | hepatic nuclear factor 1 | 4716-4730 | (−) | 1.000 | 0.798 | cGTTAatcaaaataa | 426 |
| COMP/COMP1.01 | COMP 1, cooperates with | 4717-4740 | (+) | 0.791 | 0.785 | tattttgATTAacgccgtcacagt | 427 |
| | myogenic proteins in | | | | | | |
| | multicomponent complex | | | | | | |
| CREB/ATF.01 | activating transcription | 4726-4739 | (−) | 1.000 | 0.921 | ctgTGACggcgtta | 428 |
| | factor | | | | | | |
| PAX5/PAX5.02 | B-cell-specific activating | 4733-4760 | (−) | 0.842 | 0.775 | agggactgctctaaGGCGtcactgtgac | 429 |
| | protein | | | | | | |
| PAX6/PAX6.01 | Pax-6 paired domain | 4735-4755 | (+) | 1.000 | 0.763 | cacagtgACGCcttagagcag | 430 |
| | protein | | | | | | |
| CREB/ATF.01 | activating transcription | 4737-4750 | (+) | 1.000 | 0.906 | cagTGACgccttag | 431 |
| | factor | | | | | | |
| WHZF/WHN.01 | winged helix protein, | 4738-4748 | (+) | 1.000 | 0.974 | agtgACGCctt | 432 |
| | involved in hair | | | | | | |
| | keratinization and thymus | | | | | | |
| | epithelium differentiation | | | | | | |
| FKHD/FREAC4.01 | Fork head RElated | 4756-4771 | (−) | 1.000 | 0.775 | cccgggtgAACAggga | 433 |
| | ACtivator-4 | | | | | | |
| EGRF/NGF1C.01 | nerve growth factor- | 4795-4806 | (+) | 0.763 | 0.835 | caGCGAgggtgg | 434 |
| | induced protein C | | | | | | |
| SP1F/SP1.01 | stimulating protein 1 SP | 4812-4824 | (+) | 1.000 | 0.895 | tgggGGCGgacgc | 435 |
| | 1, ubiquitous zinc finger | | | | | | |
| | transcription factor | | | | | | |
| GKLF/GKLF.01 | gut-enriched Krueppel-like | 4826-4839 | (+) | 0.950 | 0.921 | ggaaagaggaGGGG | 436 |
| | factor | | | | | | |
| PCAT/CLTR_CAAT.01 | Mammalian C-type LTR CCAAT | 4827-4851 | (−) | 0.803 | 0.780 | acCAAGgccccgcccctcctctttc | 437 |
| | box | | | | | | |
| SP1F/SP1.01 | stimulating protein 1 SP | 4834-4846 | (+) | 1.000 | 0.985 | gaggGGCGgggcc | 438 |
| | 1, ubiquitous zinc finger | | | | | | |
| | transcription factor | | | | | | |
| RREB/RREB1.01 | Ras-responsive element | 4847-4860 | (−) | 1.000 | 0.806 | cCCCAcccgaccaa | 439 |
| | binding protein 1 | | | | | | |
| TEAF/TEF1.01 | TEF-1 related muscle | 4860-4871 | (−) | 1.000 | 0.850 | ccCATTccatac | 440 |
| | factor | | | | | | |
| PAX5/PAX9.01 | zebrafish PAX9 binding | 4866-4889 | (+) | 0.866 | 0.780 | aatgGGCAgggtgggggggatggg | 441 |
| | sites | | | | | | |
| RREB/RREB1.01 | Ras-responsive element | 4868-4881 | (−) | 1.000 | 0.795 | cCCCAccctgccca | 442 |
| | binding protein 1 | | | | | | |
| EGRF/WT1.01 | Wilms Tumor Suppressor | 4874-4886 | (+) | 1.000 | 0.903 | gggTGGGggggat | 443 |
| RREB/RREB1.01 | Ras-responsive element | 4877-4890 | (−) | 1.000 | 0.796 | gCCCAtccccccca | 444 |
| | binding protein 1 | | | | | | |
| MZF1/MZF1.01 | MZF1 | 4878-4885 | (+) | 1.000 | 0.986 | gggGGGGa | 445 |
| SP1F/SP1.01 | stimulating protein 1 SP | 4884-4896 | (+) | 1.000 | 0.937 | gatgGGCGgggta | 446 |
| | 1, ubiquitous zinc finger | | | | | | |
| | transcription factor | | | | | | |
| SP1F/SP1.01 | stimulating protein 1 SP | 4900-4912 | (+) | 1.000 | 0.961 | gatgGGCGgggcc | 447 |
| | 1, ubiquitous zinc finger | | | | | | |
| | transcription factor | | | | | | |
| E2FF/E2F.03 | E2F, involved in cell | 4910-4922 | (+) | 0.806 | 0.788 | gccCGGGaaattc | 448 |
| | cycle regulation, | | | | | | |
| | interacts with RB p107 | | | | | | |
| | protein | | | | | | |
| NOLF/OLF1.01 | olfactory neuron-specific | 4915-4936 | (+) | 1.000 | 0.843 | ggaaatTCCCcggcgcgggcag | 449 |
| | factor | | | | | | |
| NFKB/NFKAPPAB.01 | NF-kappaB | 4915-4924 | (−) | 1.000 | 1 | GGGAatttcc | 450 |
| IKRS/IK1.01 | Ikaros 1, potential | 4916-4928 | (−) | 1.000 | 0.916 | gccgGGGAatttc | 451 |
| | regulator of lymphocyte | | | | | | |
| | differentiation | | | | | | |
| HEN1/HEN1.01 | HEN1 | 4944-4965 | (+) | 1.000 | 0.820 | ctggctgtCAGCtgagccgcgc | 452 |
| APAR/AP4.01 | activator protein 4 | 4950-4959 | (−) | 1.000 | 0.977 | ctCAGCtgac | 453 |
| SP1F/SP1.01 | stimulating protein 1 SP | 4964-4976 | (+) | 1.000 | 0.945 | gctgGGCGgggtc | 454 |
| | 1, ubiquitous zinc finger | | | | | | |
| | tanscription factor | | | | | | |
| EGRF/NGFIC.01 | nerve growth factor- | 5018-5029 | (−) | 0.787 | 0.802 | tgGCGGaggggg | 455 |
| | induced protein C | | | | | | |
| EGRF/NGFIC.01 | nerve growth factor- | 5024-5035 | (−) | 0.787 | 0.794 | cgGCGGtggcgg | 456 |
| | induced protein C | | | | | | |
| EGRF/NGFTC.01 | nerve growth factor- | 5030-5041 | (−) | 0.787 | 0.799 | ggGCGGcggcgg | 457 |
| | induced protein C | | | | | | |
| SPIF/SP1.01 | stimulating protein 1 SP | 5032-5044 | (−) | 1.000 | 0.898 | ggcgGGCGgcggc | 458 |
| | 1, ubiquitous zinc finger | | | | | | |
| | transcription factor | | | | | | |
| AP2F/AP2.01 | activator protein 2 | 5037-5048 | (+) | 1.000 | 0.957 | cgCCCGccggca | 459 |

As used herein, the term “cis elements capable of binding” refers to the ability of one or more of the described cis elements to specifically bind an agent. Such binding may be by any chemical, physical or biological interaction between the cis element and the agent, including, but not limited, to any covalent, steric, agostic, electronic and ionic interaction between the cis element and the agent. As used herein, the term “specifically binds” refers to the ability of the agent to bind to a specified cis element but not to wholly unrelated nucleic acid sequences. Regulatory region refers to the ability of a nucleic acid fragment, region or length to functionally perform a biological activity. The biological activity may be binding to the nucleic or specific DNA sequence. The biological activity may also modulate, enhance, inhibit or alter the transcription of a nearby coding region. The biological activity may be identified by a gel shift assay, in which binding to a nucleic acid fragment can be detected. Other methods of detecting the biological activity in a nucleic acid regulatory region are known in the art (see Current Protocols in Molecular Biology, for example).

Human transcription factor activator protein 1 (AP1) is a transcription factor that has been shown to regulate genes which are highly expressed in transformed cells such as stromelysin, c-fos, α₁-anti-trypsin and collagenase. Gutman and Wasylyk, EMBO J. 9.7: 2241-2246 (1990); Martin et al., PNAS 85: 5839-5843 (1988); Jones et al., Genes and Development 2: 267-281 (1988); Faisst and Meyer, Nucleic Acid Research 20: 3-26 (1992); Kim et al., Molecular and Cellular Biology 10: 1492-1497 (1990); Baumhueter et al., EMBO J. 7: 2485-2493 (1988). The AP1 transcription factor has been associated with genes that are activated by 12-O-tetradecanolyphorbol-13-acetate (TPA). Sequences corresponding to an upstream motif or cis element capable of binding AP1 (SEQ ID NOs: 4, 15, 18, 24, 79, 119, 122, 178, 218, 343, and 348) are located in the optineurin promoter (SEQ ID NO: 1) at the respective residues indicated in Table 2. In accordance with certain embodiments of the present invention, transcription of optineurin molecules can be effected by agents capable of altering the biochemical properties or concentration of AP1 or its homologues, including, but not limited to, the concentration of AP1 or its homologues bound to an upstream motif or cis element. Such agents can be used in the study of glaucoma pathogenesis. In another embodiment, such agents can also be used in the study of glaucoma prognosis. In another embodiment such agents can be used in the treatment of glaucoma.

A consensus sequence (GR/PR), recognized by both the glucocorticoid receptor of rat liver and the progesterone receptor from rabbit uterus, has been reported to be involved in glucocorticoid and progesterone-dependent gene expression. Von der Ahe et al., Nature 313: 706-709 (1985). Sequences corresponding to a GC/PR upstream motif or cis element (SEQ ID NOs: 70 and 312) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of glucocorticoid or progesterone or their homologues, including, but not limited to, the concentration of glucocorticoid or progesterone or their homologues bound to an GC/PR upstream motif or cis element. Such agents can be used in the study of glaucoma pathogenesis. In another embodiment, such agents can also be used in the study of glaucoma prognosis. In another embodiment such agents can be used in the treatment of glaucoma.

A consensus sequence for a vitellogenin gene-binding protein (VBP) upstream motif or cis element has been characterized. Iyer et al., Molecular and Cellular Biology 11: 4863-4875 (1991). Expression of the VBP gene commences early in liver ontogeny and is not subject to circadian control. Sequences corresponding to an upstream motif or cis element capable of binding VBP (SEQ ID NOs: 112, 155, 239, 268 and 290) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of VBP or its homologues, including, but not limited to, the concentration of VBP or its homologues bound to an VBP upstream motif or cis element. Such agents can be used in the study of glaucoma pathogenesis. In another embodiment, such agents can also be used in the study of glaucoma prognosis. In another embodiment such agents can be used in the treatment of glaucoma.

NFkB (or NFKB) is a transcription factor that is reportedly associated with a number of biological processes including T-cell activation and cytokine regulation. Lenardo et al., Cell 58: 227-229 (1989). A consensus upstream motif or cis element capable of binding NFkB has been reported. Sequences corresponding to an upstream motif or cis element capable of binding NFkB (SEQ ID NOs: 166, 186, 221, 262, 294, 332 and 450) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of NFkB 3 or its homologues, including, but not limited to, the concentration of NFkB or its homologues bound to an upstream motif or cis element. Such agents can be used in the study of glaucoma pathogenesis. In another embodiment, such agents can also be used in the study of glaucoma prognosis. In another embodiment such agents can be used in the treatment of glaucoma.

An NF1 motif or cis element has been identified which recognizes a family of at least six proteins. Courtois et al., Nucleic Acid Res. 18: 57-64 (1990); Mul et al., J. Virol. 64: 5510-5518 (1990); Rossi et al., Cell 52: 405-414 (1988); Gounari et al., EMBO J. 10: 559-566 (1990); Goyal et al., Mol. Cell Biol. 10: 1041-1048 (1990); Mermond et al., Nature 332: 557-561 (1988); Gronostajski et al., Molecular and Cellular Biology 5: 964-971 (1985); Hennighausen et al., EMBO J. 5: 1367-1371 (1986); Chodosh et al., Cell 53: 11-24 (1988). The NF1 protein will bind to an NF1 motif or cis element either as a dimer (if the motif is palindromic) or as an single molecule (if the motif is not palindromic). The NF1 protein is induced by TGFβ. Faisst and Meyer, Nucleic Acid Research 20: 3-26 (1992). Sequences corresponding to an upstream motif or cis element capable of binding NF1 (SEQ ID NOs: 117 and 194) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of NF1 or its homologues, including, but not limited to, the concentration of NF1 or its homologues bound to an upstream motif or cis element. Such agents can be used in the study of glaucoma pathogenesis. In another embodiment, such agents can also be used in the study of glaucoma prognosis. In another embodiment such agents can be used in the treatment of glaucoma.

Sequences corresponding to an upstream motif or cis element capable of binding zinc (SEQ ID NOs: 217, 223 and 417) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of zinc. Such agents can be used in the study of glaucoma pathogenesis. In another embodiment, such agents can also be used in the study of glaucoma prognosis. In another embodiment such agents can be used in the treatment of glaucoma.

Human transcription factor activator protein 2 (AP2) is a transcription factor that has been shown to bind to Sp1, nuclear factor 1 (NF1) and simian virus 40 transplantation (SV40 T) antigen binding sites. It is developmentally regulated. Williams and Tijan, Gene Dev. 5: 670-682 (1991); Mitchell et al., Genes Dev. 5: 105-119 (1991); Coutois et al., Nucleic Acid Research 18: 57-64 (1990); Comb et al., Nucleic Acid Research 18: 3975-3982 (1990); Winings et al., Nucleic Acid Research 19: 3709-3714 (1991). Sequences corresponding to an upstream motif or cis element capable of binding AP2 (SEQ ID NOs: 327, 374, and 463) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of AP2 or its homologues, including, but not limited to, the concentration of AP2 or its homologues bound to an upstream motif or cis element. Such agents may be useful in the study of glaucoma pathogenesis. In another embodiment, such agents can also be used in the study of glaucoma prognosis. In another embodiment such agents can be used in the treatment of glaucoma.

The sex-determining region of the Y chromosome gene, sry, is expressed in the fetal mouse for a brief period, just prior to testis differentiation. SRY is a DNA binding protein known to bind to a CACA-rich region in the sry gene. Vriz et al., Biochemistry and Molecular Biology International 37: 1137-1146(1995). Sequences corresponding to an upstream motif or cis element capable of binding SRY (SEQ ID NOs: 335 and 393) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of SRY or its homologues, including, but not limited to, the concentration of SRY or its homologues bound to an upstream motif or cis element. Such agents may be useful in the study of glaucoma pathogenesis. In another embodiment, such agents can also be used in the study of glaucoma prognosis. In another embodiment such agents can be used in the treatment of glaucoma.

Normal liver and differentiated hepatoma cell lines contain a hepatocyte-specific nuclear factor (HNF-1) which binds cis-acting element sequences within the promoters of the alpha and beta chains of fibrinogen and alpha 1-antitrypsin. Baumhueter et al., EMBO J. 8: 2485-2493. Sequences corresponding to an HNF-1 upstream motif or cis element (SEQ ID NOs: 126, 263, 423 and 426) are located in the optineurin promoter (SEQ ID NO: 1) at the respective residues indicated in Table 2. In accordance with the embodiments of the present invention, transcription of optineurin molecules can be effected by agents capable of altering the biochemical properties or concentration of HNF-1 or its homologues, including, but not limited to, the concentration of HNF-1 or its homologues bound to an HNF-1 upstream motif or cis element. Such agents can be used in the study of glaucoma pathogenesis. In another embodiment, such agents can also be used in the study of glaucoma prognosis. In another embodiment such agents can be used in the treatment of glaucoma.

Alu repetitive elements are unique to primates and are interspersed within the human genome with an average spacing of 4Kb. While some Alu sequences are actively transcribed by polymerase III, certain mRNA transcripts may also contain Alu-derived sequences in 5′ or 3′ untranslated regions. Jurka and Mikahanljaia, J. Mol. Evolution 32: 105-121 (1991); Claveria and Makalowski, Nature 371: 751-752 (1994). Sequences corresponding to an Alu upstream motif or cis element (SEQ ID NOs: 462 and 463) are located in the optineurin promoter (SEQ ID NO: 1) at residues 1002 through 1328 and 2288 through 2588, respectively, as depicted in FIG. 3 by a dotted line above the nucleotides.

In accordance with the embodiments of the present invention, transcription of optineurin molecules can be effected by agents capable of altering the biochemical properties or concentration of nuclear factors or their homologues, including, but not limited to, the concentration of nuclear factors or their homologues bound to an Alu upstream motif or cis element. Such agents can be used in the study of glaucoma pathogenesis. In another embodiment, such agents can also be used in the study of glaucoma prognosis. In another embodiment such agents can be used in the treatment of glaucoma.

Sequences corresponding to repeat elements (SEQ ID NOs: 460 and 461) are located in the optineurin promoter (SEQ ID NO: 1) at residues 598 through 878, and 938 through 957, respectively, as depicted in FIG. 3 by a dotted line above the nucleotides. In accordance with the embodiments of the present invention, transcription of optineurin molecules can be effected by agents capable of altering the biochemical properties or concentration of nuclear factors or their homologues, including, but not limited to, the concentration of nuclear factors or their homologues bound to a repeat element. Such agents can be used in the study of glaucoma pathogenesis. In another embodiment, such agents can also be used in the study of glaucoma prognosis. In another embodiment such agents can be used in the treatment of glaucoma.

Agents of the invention include nucleic acid molecules. In one aspect of the present invention the nucleic acid molecule is an optineurin promoter. An example of an optineurin promoter is the nucleic acid sequence set forth in SEQ ID NO: 1. In a preferred aspect of the present invention, the optineurin promoter comprises a fragment of SEQ ID NO: 1 that itself comprises at least one ATG initiation codon and includes preferably between 100 and 500 consecutive nucleotides, more preferably between 200 and 1000 consecutive nucleotides, and most preferably between 500 and 5,000 consecutive nucleotides of SEQ ID NO: 1. In a particularly preferred embodiment, the optineurin promoter fragment comprises at least 150 bases upstream of the TATA-box. More preferably, the optineurin promoter fragment is at least 15 consecutive nucleotides but not more than 1500 consecutive nucleotides of SEQ ID NO: 1 in length. In a preferred embodiment, the optineurin promoter fragment is at least 20 consecutive nucleotides but not more than 1500 consecutive nucleotides of SEQ ID NO: 1 in length.

In one embodiment the nucleic acid molecule is a DNA molecule. In another embodiment the nucleic acid molecule is an RNA molecule, more preferably an mRNA molecule. In a further embodiment the nucleic acid molecule is a double stranded molecule. In another further embodiment the nucleic acid molecule is a single stranded molecule.

In one embodiment, the nucleic acid molecule comprises one or more of the cis elements listed in Table 2. In another embodiment, the nucleic acid molecule comprises two or more of the cis elements listed in Table 2. In a further embodiment, the nucleic acid molecule comprises three, four, five, about ten, about fifteen or more, or between 3 and 3, 4 and 6, 5 and 7, 6 and 9, 10 and 15 or 20 and 30 of the cis elements listed in Table 2.

The present invention provides nucleic acid molecules that hybridize to the above-described nucleic acid molecules. Nucleic acid hybridization is a technique well known to those of skill in the art of DNA manipulation. The hybridization properties of a given pair of nucleic acids is an indication of their similarity or identity.

The nucleic acid molecules preferably hybridize, under low, moderate, or high stringency conditions, with a nucleic acid sequence selected from: (1) any of SEQ ID NOs: 3 through 463. In another aspect, the nucleic acid molecules preferably hybridize, under low, moderate, or high stringency conditions, with a nucleic acid sequence selected from the group consisting of SEQ ID NO: 1 and its complement.

The hybridization conditions typically involve nucleic acid hybridization in about 0.1X to about 10X SSC (diluted from a 20X SSC stock solution containing 3 M sodium chloride and 0.3 M sodium citrate, pH 7.0 in distilled water), about 2.5X to about 5X Denhardt's solution (diluted from a 50X stock solution containing 1% (w/v) bovine serum albumin, 1% (w/v) ficoll, and 1% (w/v) polyvinylpyrrolidone in distilled water), about 10 mg/mL to about 100 mg/mL fish sperm DNA, and about 0.02% (w/v) to about 0.1% (w/v) SDS, with an incubation at about 20° C. to about 70° C. for several hours to overnight. The stringency conditions are preferably provided by 6X SSC, 5X Denhardt's solution, 100 mg/mL fish sperm DNA, and 0.1% (w/v) SDS, with an incubation at 55° C. for several hours.

The hybridization is generally followed by several wash steps. The wash compositions generally comprise 0.1X to about 10X SSC, and 0.01% (w/v) to about 0.5% (w/v) SDS with a 15 minute incubation at about 20° C. to about 70° C. Preferably, the nucleic acid segments remain hybridized after washing at least one time in 0.1X SSC at 65° C. For example, the salt concentration in the wash step can be selected from a low stringency of about 2.0 X SSC at 50° C. to a high stringency of about 0.2 X SSC at 65° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Low stringency conditions may be used to select nucleic acid sequences with lower sequence identities to a target nucleic acid sequence. One may wish to employ conditions such as about 6.0 X SSC to about 10 X SSC, at temperatures ranging from about 20° C. to about 55° C., and preferably a nucleic acid molecule will hybridize to one or more of the above-described nucleic acid molecules under low stringency conditions of about 6.0 X SSC and about 45° C. In a preferred embodiment, a nucleic acid molecule will hybridize to one or more of the above-described nucleic acid molecules under moderately stringent conditions, for example at about 2.0 X SSC and about 65° C. In a particularly preferred embodiment, a nucleic acid molecule of the present invention will hybridize to one or more of the above-described nucleic acid molecules under high stringency conditions such as 0.2 X SSC and about 65° C.

In an alternative embodiment, the nucleic acid molecule comprises a nucleic acid sequence that is greater than 85% identical, and more preferably greater than 86, 87, 88, 89, 90, 91, 92, 93, 94, 95, 96, 97, 98, or 99% identical to a nucleic acid sequence of the present invention, preferably one selected from the group consisting of SEQ ID NO: 1, fragments of SEQ ID NO: 1 that comprise at least 20 consecutive nucleotides but not more than 1500 consecutive nucleotides of the sequence of SEQ ID NO: 1, and complements thereof.

The percent identity is preferably determined using the “Best Fit” or “Gap” program of the Sequence Analysis Software Package™ (Version 10; Genetics Computer Group, Inc., University of Wisconsin Biotechnology Center, Madison, Wis.). “Gap” utilizes the algorithm of Needleman and Wunsch to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman. The percent identity calculations may also be performed using the Megalign program of the LASERGENE bioinformatics computing suite (default parameters, DNASTAR Inc., Madison, Wis.). The percent identity is most preferably determined using the “Best Fit” program using default parameters.

The present invention also provides nucleic acid molecule fragments that hybridize to the above-described nucleic acid molecules and complements thereof, fragments of nucleic acid molecules that exhibit greater than 80%, 85%, 90%, 95% or 99% sequence identity with a nucleic acid molecule of the present invention.

Fragment nucleic acid molecules may consist of significant portion(s) of, or indeed most of, the nucleic acid molecules of the invention. In an embodiment, the fragments are between 3000 and 1000 consecutive nucleotides, 1800 and 150 consecutive nucleotides, 1500 and 500 consecutive nucleotides, 1300 and 250 consecutive nucleotides, 1000 and 200 consecutive nucleotides, 800 and 150 consecutive nucleotides, 500 and 100 consecutive nucleotides, 300 and 75 consecutive nucleotides, 100 and 50 consecutive nucleotides, 50 and 25 consecutive nucleotides, or 20 and 10 consecutive nucleotides long of a nucleic molecule of the present invention.

In another embodiment, the fragment comprises at least 20, 30, 40, 50, 60, 70, 80, 90, 100, 150, 200, 250, 500, or 750 consecutive nucleotides of a nucleic acid sequence of the present invention. In another embodiment, the fragment comprises at least 12, 15, 18, 20, 25, 50, 75, 100, 125, 150, 200, 250, 300, 350, 400, 450 but not more 500, 550, 600, 650, 700, 750, 800, 1000, 1200, 1400, or 1500 consecutive nucleotides of a nucleic acid sequence selected from the group consisting of SEQ ID NO: 1 and complements thereof.

Any of a variety of methods may be used to obtain one or more of the above-described nucleic acid molecules. Automated nucleic acid synthesizers may be employed for this purpose. In lieu of such synthesis, the disclosed nucleic acid molecules may be used to define a pair of primers that can be used with the polymerase chain reaction to amplify and obtain any desired nucleic acid molecule or fragment.

Short nucleic acid sequences having the ability to specifically hybridize to complementary nucleic acid sequences may be produced and utilized in the present invention, e.g., as probes to identify the presence of a complementary nucleic acid sequence in a given sample. Alternatively, the short nucleic acid sequences may be used as oligonucleotide primers to amplify or mutate a complementary nucleic acid sequence using PCR technology. These primers may also facilitate the amplification of related complementary nucleic acid sequences (e.g., related sequences from other species).

Use of these probes or primers may greatly facilitate the identification of transgenic cells or organisms which contain the presently disclosed promoters and structural nucleic acid sequences. Such probes or primers may also, for example, be used to screen cDNA or genomic libraries for additional nucleic acid sequences related to or sharing homology with the presently disclosed promoters and structural nucleic acid sequences. The probes may also be PCR probes, which are nucleic acid molecules capable of initiating a polymerase activity while in a double-stranded structure with another nucleic acid.

A primer or probe is generally complementary to a portion of a nucleic acid sequence that is to be identified, amplified, or mutated and of sufficient length to form a stable and sequence-specific duplex molecule with its complement. The primer or probe preferably is about 10 to about 200 nucleotides long, more preferably is about 10 to about 100 nucleotides long, even more preferably is about 10 to about 50 nucleotides long, and most preferably is about 14 to about 30 nucleotides long.

The primer or probe may, for example without limitation, be prepared by direct chemical synthesis, by PCR (U.S. Pat. Nos. 4,683,195 and 4,683,202), or by excising the nucleic acid specific fragment from a larger nucleic acid molecule. Various methods for determining the structure of PCR probes and PCR techniques exist in the art. Computer-generated searches using programs such as Primer3 (www-genome.wi.mit. edu/cgi-bin/primer/primer3.cgi), STSPipeline (www-genome.wi.mit.edu/cgi-bin/www-STS_Pipeline), or GeneUp (Pesole et al., BioTechniques 25:112-123, 1998), for example, can be used to identify potential PCR primers.

Nucleic acid agents of the present invention may also be employed to obtain other optineurin nucleic acid molecules. Such molecules include the optineurin-encoding nucleic acid molecules of non-human animals (particularly cats, monkeys, rodents and dogs), fragments thereof, and promoters and flanking sequences. Such molecules can readily be obtained by using the above-described primers to screen cDNA or genomic libraries obtained from non-human species. Methods for forming such libraries are known in the art.

Any of the nucleic acid agents of the invention may be linked with additional nucleic acid sequences to encode fusion proteins. The additional nucleic acid sequence preferably encodes at least one amino acid, peptide, or protein. Many possible fusion combinations exist. For instance, the fusion protein may provide a “tagged” epitope to facilitate detection of the fusion protein, such as GST, GFP, FLAG, or polyHIS. Such fusions preferably encode between 1 and 50 amino acids, more preferably between 5 and 30 additional amino acids, and even more preferably between 5 and 20 amino acids.

Alternatively, the fusion may provide regulatory, enzymatic, cell signaling, or intercellular transport functions. For example, a sequence encoding a signal peptide may be added to direct a fusion protein to a particular organelle within a eukaryotic cell. Such fusion partners preferably encode between 1 and 1000 additional amino acids, more preferably between 5 and 500 additional amino acids, and even more preferably between 10 and 250 amino acids.

The above-described protein or peptide molecules may be produced via chemical synthesis, or more preferably, by expression in a suitable bacterial or eukaryotic host. Suitable methods for expression are described by Sambrook et al., supra, or similar texts. Fusion protein or peptide molecules of the invention are preferably produced via recombinant means. These proteins and peptide molecules may be derivatized to contain carbohydrate or other moieties (such as keyhole limpet hemocyanin, etc.).

B. Recombinant Vectors and Constructs

Exogenous genetic material may be transferred into a host cell by use of a vector or construct designed for such a purpose. Preferred exogenous genetic material is a nucleic acid molecule of the present invention, more preferred exogenous genetic material is an optineurin promoter sequence, and even more preferred exogenous genetic material is a nucleic acid molecule comprising SEQ ID NO: 1.

Any of the nucleic acid sequences described above may be provided in a recombinant vector. As used herein, “vector” refers to a plasmid, cosmid, bacteriophage, BAC, YAC, or virus that carries exogenous DNA into a host organism. A plasmid may be a linear or a closed circular plasmid. The vector system may be a single vector or plasmid or two or more vectors or plasmids which together contain the total DNA to be introduced into the genome of the host. Means for preparing recombinant vectors are well known in the art.

Vectors suitable for replication in mammalian cells may include viral replicons, or sequences which insure integration of the appropriate sequences encoding HCV epitopes into the host genome. For example, another vector used to express foreign DNA is vaccinia virus. Such heterologous DNA is generally inserted into a gene which is non-essential to the virus, for example, the thymidine kinase gene (tk), which also provides a selectable marker. Expression of the HCV polypeptide then occurs in cells or animals which are infected with the live recombinant vaccinia virus.

In general, plasmid vectors containing replicon and control sequences that are derived from species compatible with the host cell are used in connection with bacterial hosts. The vector ordinarily carries a replication site, as well as marking sequences that are capable of providing phenotypic selection in transformed cells. For example, E. coli is typically transformed using pBR322, which contains genes for ampicillin and tetracycline resistance and thus provides easy means for identifying transformed cells. The pBR322 plasmid, or other microbial plasmid or phage, also generally contains, or is modified to contain, promoters that can be used by the microbial organism for expression of the selectable marker genes.

A construct or vector may include a promoter, e.g., a recombinant vector typically comprises, in a 5′ to 3′ orientation: a promoter to direct the transcription of a nucleic acid sequence of interest and a nucleic acid sequence of interest. Suitable promoters include, but are not limited to, those described herein. The recombinant vector may further comprise a 3′ transcriptional terminator, a 3′ polyadenylation signal, other untranslated nucleic acid sequences, transit and targeting nucleic acid sequences, selectable markers, enhancers, and operators, as desired.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. For autonomous replication, the vector may further comprise an origin of replication enabling the vector to replicate autonomously in the host cell in question. Alternatively, the vector may be one which, when introduced into the cell, is integrated into the genome and replicated together with the chromosome(s) into which it has been integrated. This integration may be the result of homologous or non-homologous recombination.

Integration of a vector or nucleic acid into the genome by homologous recombination, regardless of the host being considered, relies on the nucleic acid sequence of the vector. Typically, the vector contains nucleic acid sequences for directing integration by homologous recombination into the genome of the host. These nucleic acid sequences enable the vector to be integrated into the host cell genome at a precise location or locations in one or more chromosomes. To increase the likelihood of integration at a precise location, there should be preferably two nucleic acid sequences that individually contain a sufficient number of nucleic acids, preferably 400 bp to 1500 bp, more preferably 800 bp to 1000 bp, which are highly homologous with the corresponding host cell target sequence. This enhances the probability of homologous recombination. These nucleic acid sequences may be any sequence that is homologous with a host cell target sequence and, furthermore, may or may not encode proteins.

Promoters

In addition to the optineurin promoters described herein, other promoter sequences can be utilized in a vector or other nucleic acid molecule. In a preferred aspect, the promoter is operably linked to another nucleic acid molecule. The promoters may be selected on the basis of the cell type into which the vector will be inserted. The promoters may also be selected on the basis of their regulatory features, e.g., enhancement of transcriptional activity, inducibility, tissue specificity, and developmental stage-specificity. Additional promoters that may be utilized are described, for example, in Bernoist and Chambon, Nature 290:304-310 (1981); Yamamoto et al., Cell 22:787-797 (1980); Wagner et al., PNAS 78:1441-1445 (1981); Brinster et al., Nature 296:39-42 (1982).

Suitable promoters for mammalian cells are also known in the art and include viral promoters, such as those from Simian Virus 40 (SV40), Rous sarcoma virus (RSV), adenovirus (ADV), cytomegalovirus (CMV), and bovine papilloma virus (BPV), as well as mammalian cell-derived promoters. Other preferred promoters include the hematopoietic stem cell-specific, e.g., CD34, glucose-6-phosphotase, interleukin-1 alpha, CD11c integrin gene, GM-CSF, interleukin-5R alpha, interleukin-2, c-fos, h-ras and DMD gene promoters. Other promoters include the herpes thymidine kinase promoter, and the regulatory sequences of the metallothionein gene.

Inducible promoters suitable for use with bacteria hosts include the β-lactamase and lactose promoter systems, the arabinose promoter system, alkaline phosphatase, a tryptophan (trp) promoter system and hybrid promoters such as the tac promoter. However, other known bacterial inducible promoters are suitable. Promoters for use in bacterial systems also generally contain a Shine-Dalgarno sequence operably linked to the DNA encoding the polypeptide of interest.

Additional Nucleic Acid Sequences of Interest

The recombinant vector may also contain one or more additional nucleic acid sequences of interest. These additional nucleic acid sequences may generally be any sequences suitable for use in a recombinant vector. Such nucleic acid sequences include, without limitation, any of the nucleic acid sequences, and modified forms thereof, described above. The additional nucleic acid sequences may also be operably linked to any of the above described promoters. The one or more additional nucleic acid sequences may each be operably linked to separate promoters. Alternatively, the additional nucleic acid sequences may be operably linked to a single promoter (i.e. a single operon).

The additional nucleic acid sequences include, without limitation, those encoding gene products which are toxic to a cell such as the diptheria A gene product.

Alternatively, the additional nucleic acid sequence may be designed to down-regulate a specific nucleic acid sequence. This is typically accomplished by operably linking the additional nucleic acid sequence, in an antisense orientation, with a promoter. One of ordinary skill in the art is familiar with such antisense technology. Any nucleic acid sequence may be negatively regulated in this manner. Preferable target nucleic acid sequences include SEQ ID NOs: 3 through 463.

Selectable and Screenable Markers

A vector or construct may also include a selectable marker. Selectable markers may also be used to select for organisms or cells that contain the exogenous genetic material. Examples of such include, but are not limited to: a neo gene, which codes for kanamycin resistance and can be selected for using kanamycin, GUS, green fluorescent protein (GFP), neomycin phosphotransferase II (nptII), luciferase (LUX), or an antibiotic resistance coding sequence.

A vector or construct may also include a screenable marker. Screenable markers may be used to monitor expression. Exemplary screenable markers include: a β-glucuronidase or uidA gene (GUS) which encodes an enzyme for which various chromogenic substrates are known; a β-lactamase gene, a gene which encodes an enzyme for which various chromogenic substrates are known (e.g., PADAC, a chromogenic cephalosporin); a luciferase gene; a tyrosinase gene, which encodes an enzyme capable of oxidizing tyrosine to DOPA and dopaquinone which in turn condenses to melanin; and α-galactosidase, which will turn a chromogenic α-galactose substrate.

Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins which are detectable, (e.g., by ELISA), or small active enzymes which are detectable in extracellular solution (e.g., α-amylase, β-lactamase, phosphinothricin transferase). Other possible selectable and/or screenable marker genes will be apparent to those of skill in the art.

C. Transgenic Organisms, Transformed and Transfected Host Cells

One or more of the nucleic acid molecules or recombinant vectors of the invention may be used in transformation or transfection. For example, exogenous genetic material may be transferred into a cell or organism. In a preferred embodiment, the exogenous genetic material includes a nucleic acid molecule of the present invention, preferably a nucleic acid molecule of an optineurin promoter. In another preferred embodiment, the nucleic acid molecule has a sequence selected from the group consisting of SEQ ID NO: 1, fragments of SEQ ID NO: 1 that comprise at least 20 consecutive nucleotides but not more than 1500 consecutive nucleotides of the sequence of SEQ ID NO: 1, and complements thereof.

The invention is also directed to transgenic or transfected organisms and transformed or transfected host cells which comprise, in a 5′ to 3′ orientation, a promoter operably linked to a heterologous nucleic acid sequence of interest. Additional nucleic acid sequences may be introduced into the organism or host cell, such as 3′ transcriptional terminators, 3′ polyadenylation signals, other untranslated nucleic acid sequences, signal or targeting sequences, selectable markers, enhancers, and operators. Preferred nucleic acid sequences of the present invention, including recombinant vectors, structural nucleic acid sequences, promoters, and other regulatory elements, are described herein. Another embodiment of the invention is directed to a method of producing such transgenic organisms which generally comprises the steps of selecting a suitable organism, transforming the organism with a recombinant vector, and obtaining the transformed organism.

Transfer of a nucleic acid that encodes a protein can result in expression or overexpression of that protein in a transformed cell or transgenic organism. One or more of the proteins or fragments thereof encoded by nucleic acid molecules of the invention may be overexpressed in a transformed cell or transgenic organism. Such expression or overexpression may be the result of transient or stable transfer of the exogenous genetic material.

The expressed protein may be detected using methods known in the art that are specific for the particular protein or fragment. These detection methods may include the use of specific antibodies, formation of an enzyme product, or disappearance of an enzyme substrate. For example using the antibodies to the protein. The techniques of enzyme assay and immunoassay are well known to those skilled in the art.

The resulting protein may be recovered by methods known in the arts. For example, the protein may be recovered from the nutrient medium by procedures including, but not limited to, centrifugation, filtration, extraction, spray-drying, evaporation, or precipitation. The recovered protein may then be further purified by a variety of chromatographic procedures, e.g., ion exchange chromatography, gel filtration chromatography, affinity chromatography, or the like. Reverse-phase high performance liquid chromatography (RP-HPLC), optionally employing hydrophobic RP-HPLC media, e.g., silica gel, further purify the protein. Combinations of methods and means can also be employed to provide a substantially purified recombinant polypeptide or protein.

Technology for introduction of nucleic acids into cells is well known to those of skill in the art. Common methods include chemical methods, microinjection, electroporation (U.S. Pat. No. 5,384,253), particle acceleration, viral vectors, and receptor-mediated mechanisms. Fungal cells may be transformed by a process involving protoplast formation, transformation of the protoplasts and regeneration of the cell wall. The various techniques for transforming mammalian cells are also well known.

There are many methods for introducing transforming DNA segments into cells, but not all are suitable for delivering DNA to eukaryotic cells. Suitable methods include virtually any method by which DNA can be introduced into a cell, such as by direct delivery of DNA, by desiccation/inhibition-mediated DNA uptake, by electroporation, by agitation with silicon carbide fibers, by acceleration of DNA coated particles, by chemical transfection, by lipofection or liposome-mediated transfection, by calcium chloride-mediated DNA uptake, etc. In certain embodiments, acceleration methods are preferred and include, for example, microprojectile bombardment and the like.

A transformed or transfected host cell may generally be any cell which is compatible with the present invention. A transformed or transfected host organism or cell can be or derived from a cell or organism such as a mammalian cell, mammal, fish cell, fish, bird cell, bird, fungal cell, fungus, or bacterial cell. Preferred host and transformants include: fungal cells such as Aspergillus, yeasts, mammals, particularly murine, bovine and porcine, insects, bacteria, and algae. Methods to transform and transfect such cells or organisms are known in the art. See, e.g., EP 238023; Becker and Guarente, in: Abelson and Simon (eds.), Guide to Yeast Genetics and Molecular Biology, Methods Enzymol. 194: 182-187, Academic Press, Inc., New York; Bennett and LaSure (eds.), More Gene Manipulations in Fungi, Academic Press, Calif., 1991; Hinnen et al., PNAS 75:1920, 1978; Ito et al., J. Bacteriology 153:163, 1983; Malardier et al., Gene 78:147-156, 1989; Yelton et al., PNAS 81:1470-1474, 1984. Mammalian cell lines available as hosts for expression are known in the art and include many immortalized cell lines available from the American Type Culture Collection (ATCC, Manassas, Va.), such as HeLa cells, Chinese hamster ovary (CHO) cells, baby hamster kidney (BHK) cells and a number of other cell lines. Non-limiting examples of suitable mammalian host cell lines include those shown below in Table 3.

TABLE 3
Mammalian Host Cell Lines

| Host Cell | Origin | Source |
|---|---|---|
| HepG-2 | Human Liver Hepatoblastoma | ATCC HB 8065 |
| CV-1 | African Green Monkey Kidney | ATCC CCL 70 |
| LLC-MK₂ | Rhesus Monkey Kidney | ATCC CCL 7 |
| 3T3 | Mouse Embryo Fibroblasts | ATCC CCL 92 |
| AV12-664 | Syrian Hamster | ATCC CRL 9595 |
| HeLa | Human Cervix Epitheloid | ATCC CCL 2 |
| RPMI8226 | Human Myeloma | ATCC CCL 155 |
| H4IIEC3 | Rat Hepatoma | ATCC CCL 1600 |
| C127I | Mouse Fibroblast | ATCC CCL 1616 |
| 293 | Human Embryonal Kidney | ATCC CRL 1573 |
| HS-Sultan | Human Plasma Cell Plasmocytoma | ATCC CCL 1484 |
| BHK-21 | Baby Hamster Kidney | ATCC CCL 10 |
| HTM | Human Trabecular Meshwork | Stamer* |
| hTERT-RPE1 | Human Retinal Pigment | Clontech^{†} |
| | Epithelial Cells | |
| HCE | Human Corneal Epithelium | LSU Eye Center^{‡} |
| B-3 | Human Eye | CRL-11421 |
| CHO-K1 | Chinese Hamster Ovary | ATCC CCL 61 |

(*Stamer, Current Eye Research 20: 347-350 (2000). ^{†}Clontech, Palo Alto, California. ^{‡}LSU Eye Center, New Orleans, LA.)

A fungal host cell may, for example, be a yeast cell, a fungi, or a filamentous fungal cell. In one embodiment, the fungal host cell is a yeast cell, and in a preferred embodiment, the yeast host cell is a cell of the species of Candida, Kluyveromyces, Saccharomyces, Schizosaccharomyces, Pichia and Yarrowia. In another embodiment, the fungal host cell is a filamentous fungal cell, and in a preferred embodiment, the filamentous fungal host cell is a cell of the species of Acremonium, Aspergillus, Fusarium, Humicola, Myceliophthora, Mucor, Neurospora, Penicillium, Thielavia, Tolypocladium and Trichoderma.

Suitable host bacteria include archaebacteria and eubacteria, especially eubacteria and most preferably Enterobacteriaceae. Examples of useful bacteria include Escherichia, Enterobacter, Azotobacter, Erwinia, Bacillus, Pseudomonas, Klebsiella, Proteus, Salmonella, Serratia, Shigella, Rhizobia, Vitreoscilla and Paracoccus. Suitable E. coli hosts include E. coli W3110 (ATCC 27325), E. coli 294 (ATCC 31446), E. coli B and E. coli X1776 (ATCC 31537) (American Type Culture Collection, Manassas, Va.). Mutant cells of any of the above-mentioned bacteria may also be employed. These hosts may be used with bacterial expression vectors such as E. coli cloning and expression vector Bluescript™ (Stratagene, La Jolla, Calif.); pIN vectors (U.S. Pat. No. 5,426,050), and pGEX vectors (Promega, Madison, Wis.), which may be used to express foreign polypeptides as fusion proteins with glutathione S-transferase (GST).

Preferred insect host cells are derived from Lepidopteran insects such as Spodoptera frugiperda or Trichoplusia ni. The preferred Spodoptera frugiperda cell line is the cell line Sf9 (ATCC CRL 1711). Other insect cell systems, such as the silkworm B. mori may also be used. These host cells are preferably used in combination with Baculovirus expression vectors (BEVs), which are recombinant insect viruses in which the coding sequence for a chosen foreign gene has been inserted behind a baculovirus promoter in place of the viral gene, e.g., polyhedrin (U.S. Pat. No. 4,745,051).

One aspect of the present invention relates to transgenic non-human animals having germline and/or somatic cells in which the biological activity of one or more genes are altered by a chromosomally incorporated transgene. In a preferred embodiment, the transgene encodes an antisense transcript which, when transcribed from the transgene, hybridizes with a portion of the optineurin promoter sequence, and inhibits expression of the optineurin gene.

In one embodiment, the present invention provides a desired non-human animal or an animal (including human) cell which contains a predefined, specific and desired alteration rendering the non-human animal or animal cell predisposed to glaucoma. Specifically, the invention pertains to a genetically altered non-human animal (most preferably, a mouse), or a cell (either non-human animal or human) in culture, that expresses an antisense sequence directed to the optineurin promoter. Animals that express an antisense sequence directed to the optineurin promote may exhibit a higher susceptibility to glaucoma or other ophthalmic disorders. By way of example, a genetically altered mouse of this type is able to serve as a model for hereditary glaucomas and as a test animal for glaucoma studies. Non-human animals or animal cells that express an antisense sequence directed to the optineurin promoter are able to serve as a glaucoma model. The invention additionally pertains to the use of such non-human animals or animal cells. Furthermore, it is contemplated that cells of the transgenic animals of the present invention can include other transgenes.

D. Inhibition of Gene Expression

In one aspect the activity or expression of an optineurin molecule is reduced by affecting the activity of the optineurin promoter. In a preferred aspect, the activity or expression of an optineurin molecule is reduced by greater than 50%, 60%, 70%, 80% or 90% by the introduction into a recipient cell or host of an agent of the invention.

Antisense approaches are a way of preventing or reducing gene function by targeting the genetic material. The objective of the antisense approach is to use a sequence complementary to the target gene or its promoter to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes.

Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression. An antisense vector can be constructed by standard procedures and introduced into cells by transformation, transfection, electroporation, microinjection, infection, etc. The type of transformation and choice of vector will determine whether expression is transient or stable. The promoter used for the antisense gene may influence the level, timing, tissue, specificity, or inducibility of the antisense inhibition.

One aspect of the invention relates to the use of nucleic acids, e.g., SEQ ID NOs: 1 through 463, fragments thereof, or sequences complementary thereto, in antisense therapy. As used herein, antisense therapy refers to administration or in situ generation of oligonucleotide molecules or their derivatives which specifically hybridize (e.g., bind) under physiological conditions with the cellular mRNA and/or genomic DNA, thereby inhibiting transcription and/or translation of that gene. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell, causes inhibition of expression by hybridizing with the mRNA and/or genomic sequences of a subject nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al., BioTechniques 6:958-976 (1988); and Stein et al., Cancer Res 48:2659-2668 (1988). With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See Wagner, Nature 372:333 (1994). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the invention. Whether designed to hybridize to the 5′, 3′, or coding region of subject mRNA, antisense nucleic acids should be at least six nucleotides in length, and are preferably less than about 100 and more preferably less than about 50, 25, 17 or 10 nucleotides in length.

Regardless of the choice of target sequence, it is preferred that in vitro studies are first performed to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., PNAS 86:6553-6556 (1989); Lemaitre et al., PNAS 84:648-652 (1987); WO 88/09810) or the blood-brain barrier (see, e.g., WO 89/10134), hybridization-triggered cleavage agents (See, e.g., Krol et al., BioTechniques 6:958-976 (1988)), or intercalating agents (see, e.g., Zon, Pharm. Res. 5:539-549 (1988)). To this end, the oligonucleotide may be conjugated to another molecule, e.g., a peptide, hybridization triggered cross-linking agent, transport agent, hybridization-triggered cleavage agent, etc.

Antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Antisense oligonucleotides may also comprise at least one modified sugar moiety selected from the group including but not limited to arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al., PNAS 93:14670 (1996) and in Eglom et al., Nature 365:566 (1993). One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., Nucl. Acids Res. 15:6625-6641 (1987)). The oligonucleotide is a 2′-O-methylribonucleotide (Inoue et al., Nucl. Acids Res. 15:6131-12148 (1987)), or a chimeric RNA-DNA analogue (Inoue et al., FEBS Lett. 215:327-330 (1987)).

Antisense molecules can be delivered to cells which express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous transcripts and thereby prevent translation of the target mRNA. For example, a vector can be introduced in viva such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art, and can be plasmid, viral, or others known in the art for replication and expression in mammalian cells.

Expression of the sequence encoding the antisense RNA can be by any promoter known in the art to act in mammalian, preferably human cells. Such promoters can be inducible or constitutive. Such promoters include but are not limited to: the SV40 early promoter region, the promoter contained in the 3′ long terminal repeat of Rous sarcoma virus, the herpes thymidine kinase promoter, the regulatory sequences of the metallothionein gene, etc. Any type of plasmid, cosmid, BAC, YAC or viral vector can be used to prepare the recombinant DNA construct which can be introduced directly into the tissue site; e.g., the choroid plexus or hypothalamus. Alternatively, viral vectors can be used which selectively infect the desired tissue (e.g., for brain, herpesvirus vectors may be used), in which case administration may be accomplished by another route (e.g., systemically).

Antisense RNA, DNA, and ribozyme molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Endogenous gene expression can be reduced by inactivating or “knocking out” the gene or its promoter using targeted homologous recombination. (E.g. see Smithies et al., Nature 317:230-234 (1985); Thomas & Capecchi, Cell 51:503-512 (1987); Thompson et al., Cell 5:313-321(1989)). For example, a mutant, non-functional gene (or a completely unrelated DNA sequence) flanked by DNA homologous to the endogenous gene (either the coding regions or regulatory regions of the gene) can be used, with or without a selectable marker and/or a negative selectable marker, to transfect cells that express that gene in vivo. Insertion of the DNA construct, via targeted homologous recombination, results in inactivation of the gene.

E. Pharmaceutical Compositions

Pharmaceutical compositions can comprise polynucleotides of the present invention. The pharmaceutical compositions will comprise a therapeutically effective amount of nucleic acid molecules of the present invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example, an optineurin promoter molecule or fragments thereof, antibodies of an optineurin promoter molecule, agonists, antagonists or inhibitors of the optineurin promoter, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. For purposes of the present invention, an effective dose will be from about 0.01 mg/kg to 50 mg/kg or 0.05 mg/kg to about 10 mg/kg of the DNA constructs in the individual to which it is administered.

There is a wide variety of suitable formulations of pharmaceutical compositions of the present invention (see, e. g., Remington's Pharmaceutical Sciences, 17th ed. 1985). Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable carriers in therapeutic compositions may contain liquids such as water, saline, glycerol and ethanol. Other pharmaceutically acceptable carriers include, but are not limited to, gum arabic, vegetable oils, benzyl alcohols, polyethylene glycols, gelatin, carbohydrates such as lactose, amylose or starch, dextrose, magnesium stearate, talc, silicic acid, viscous paraffin, fatty acid esters, hydroxmethylcellulose, polyvinyl pyrrolidone, as well as combinations thereof. Additionally, auxiliary substances, such as wetting or emulsifying agents, lubricants, preservatives, stabilizers, pH buffering substances, coloring, flavoring and the like, may be present in such vehicles.

Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier. The formulations of compounds can be presented in unit-dose or multi-dose sealed containers, such as ampules and vials. Solutions and suspensions can be prepared from sterile powders, granules, and tablets.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. Pharmaceutically acceptable excipients can also be used therein.

The invention also provides a pharmaceutical pack or kit comprising one or more containers filled with one or more of the ingredients of the pharmaceutical compositions that can be used in the methods of treatment. Optionally associated with such container(s) can be a notice or leaflet in the form prescribed by a governmental agency regulating the manufacture, use or sale of pharmaceuticals or biological products, which notice or leaflet reflects approval by the agency of manufacture, use, or sale for human administration. The pack or kit can contain a leaflet or be labeled with information regarding mode of administration, sequence of drug administration (e.g., separately, sequentially, or concurrently), or the like. The pack or kit may also contain means for reminding the patient to take the therapy. The pack or kit may be a single unit dosage, a plurality of unit dosages, or a combination therapy.

In particular, the agents can be separated, mixed together in any combination, or present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Delivery Methods

Once formulated, the pharmaceuticals compositions of the invention can be (1) administered directly to the subject; (2) delivered ex vivo, to cells derived from the subject; or (3) delivered in vitro for expression of recombinant proteins.

Methods for direct delivery of the compositions include, but are not limited to, subcutaneous, intraperitoneal, intraocular, intranasal, intravenous, intramuscular, intradermal, oral, intranasal, topical, intravesical, intrathecal, or delivered to the interstitial space of a tissue. In a preferred embodiment, the composition is introduced intraocularly by, for example, eye drops. Other modes of administration include oral and pulmonary administration, suppositories, and transdermal applications, needles, and gene guns or hyposprays. Dosage treatment may be a single dose schedule or a multiple dose schedule.

Methods for the ex vivo delivery and reimplantation of transformed cells into a subject are known in the art and described in e.g., WO 93/14778. Examples of cells useful in ex vivo applications include, for example, stem cells, particularly hematopoetic, lymph cells, macrophages, dendritic cells, or tumor cells, and trabecular meshwork cells, particularly human trabecular meshwork cells.

Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Preparation of antisense polypeptides is discussed above. Both the dose of the antisense composition and the means of administration are determined based on the specific qualities of the therapeutic composition, the condition, age, and weight of the patient, the progression of the disease, and other relevant factors. Administration of the therapeutic antisense agents of the invention includes local or systemic administration, including injection, oral administration, particle gun or catheterized administration, and topical administration. Preferably, the therapeutic antisense composition contains an expression construct comprising a promoter and a polynucleotide segment of at least about 12, 22, 25, 30, or 35 contiguous nucleotides of the antisense strand of a nucleic acid. Within the expression construct, the polynucleotide segment is located downstream from the promoter, and transcription of the polynucleotide segment initiates at the promoter.

Receptor-mediated targeted delivery of therapeutic compositions containing an antisense polynucleotide, subgenomic polynucleotides, or antibodies to specific tissues is also used. Receptor-mediated DNA delivery techniques are described in, for example, Findeis et al., Trends in Biotechnol. (1993) 11:202-205; Chiou et al., (1994) Gene Therapeutics: Methods And Applications Of Direct Gene Transfer (J. A. Wolff, ed.); Wu & Wu, J. Biol. Chem. (1988) 263:621-24; Wu et al., J. Biol. Chem. (1994) 269:542-46; Zenke et al., PNAS (1990) 87:3655-59; Wu et al., J. Biol. Chem. (1991) 266:338-42. Preferably, receptor-mediated targeted delivery of therapeutic compositions containing antibodies of the invention is used to deliver the antibodies to specific tissue.

Therapeutic compositions containing antisense subgenomic polynucleotides are administered in a range of about 100 ng to about 200 mg of DNA for local administration in a gene therapy protocol. Concentration ranges of about 500 ng to about 50 mg, about 1 mg to about 2 mg, about 5 mg to about 500 mg, and about 20 mg to about 100 mg of DNA can also be used during a gene therapy protocol. Factors such as method of action and efficacy of transformation and expression are considerations which will affect the dosage required for ultimate efficacy of the antisense subgenomic nucleic acids. Where greater expression is desired over a larger area of tissue, larger amounts of antisense subgenomic nucleic acids or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect.

For genes encoding polypeptides or proteins with anti-inflammatory activity, suitable use, doses, and administration are described in U.S. Pat. No. 5,654,173. Therapeutic agents also include antibodies to proteins and polypeptides encoded by the subject nucleic acids, as described in U.S. Pat. No. 5,654,173.

Gene Delivery

The therapeutic nucleic acids of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0415731; EP 0345242; WO 90/07936; WO 94/03622; WO 93/25698; WO 93/25234; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. Nos. 5,219,740 and 4,777,127; and GB Patent No. 2,200,651. Preferred recombinant retroviruses include those described in WO 91/02805.

Packaging cell lines suitable for use with the above-described retroviral vector constructs may be readily prepared (WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. (1988) 166:154-165; and Flotte et al., PNAS 90:10613-10617 (1993).

Representative examples of adenoviral vectors include those described by Berkner, Biotechniques 6:616-627 (1988); Rosenfeld et al., Science 252:431-434 (1991); WO 93/19191; Kolls et al., PNAS 91:215-219 (1994); Kass-Eisler et al., PNAS 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1991). Exemplary adenoviral gene therapy vectors employable in this invention also include those described in WO 94/12649, WO 93/03769, WO 93/19191, WO 94/28938, WO 95/11984 and WO 95/00655. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone (Curiel, Hum. Gene Ther. 3:147-154 (1992)); ligand linked DNA (Wu, J. Biol. Chem. 264:16985-16987 (1989)); eukaryotic cell delivery vehicles cells (U.S. Pat. No. 6,287,792); deposition of photopolymerized hydrogel materials; hand-held gene transfer particle gun (U.S. Pat. No. 5,149,655); ionizing radiation (U.S. Pat. No. 5,206,152; WO 92/11033); and nucleic charge neutralization or fusion with cell membranes. Additional approaches are described in Philip, Mol. Cell Biol. 14:2411-2418 (1994), and in Woffendin et al., PNAS 91:11581-11585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, WO 95/13796, WO 94/23697, WO 91/14445, and EP 0524968.

F. Diagnostic and Prognostic Assays

Agents of the present invention can be utilized in methods to determine, for example, without limitation, the presence or absence of a nucleic acid molecule in a sample, and the level of nucleic acid molecule in a sample. Moreover, agents of the present invention can be utilized in methods for diagnosing glaucoma, methods for prognosing glaucoma, and methods for predicting a predisposition to glaucoma.

As used herein, the “Expression Response” manifested by a cell or tissue of an organism is said to be “altered” if it differs from the Expression Response of cells or tissues not exhibiting the phenotype. To determine whether a Expression Response is altered, the Expression Response manifested by the cell or tissue of the organism exhibiting the phenotype is compared with that of a similar cell or tissue sample of an organism not exhibiting the phenotype. As will be appreciated, it is not necessary to re-determine the Expression Response of the cell or tissue sample of organisms not exhibiting the phenotype each time such a comparison is made; rather, the Expression Response of a particular organism may be compared with previously obtained values of normal organisms.

Also as used herein, a “tissue sample” is any sample that comprises more than one cell. In a preferred aspect, a tissue sample comprises cells that share a common characteristic (e.g. derived from neurons, epidermis, muscle etc.). Preferred cells and tissue samples may be derived from bodily fluids including glaucomatous cell extract, fluid from the anterior chamber of the eye, blood, lymph, serum, amniotic fluid, and cerebrospinal fluid, or from skin, muscle, buccal or conjunctival mucosa, placenta, gastrointestinal tract or other organs. A test sample may be derived from adults, juveniles, and fetuses. Test samples from fetal cells or tissue can be obtained by appropriate methods, such as by amniocentesis or chorionic villus sampling. In a preferred embodiment, a sample is derived from bodily fluids such as glaucomatous cell extract, fluid from the anterior chamber of the eye, blood, lymph, and serum.

A number of methods can be used to compare the expression response between two or more samples of cells or tissue. These methods include hybridization assays, such as northerns, RNAse protection assays, and in situ hybridization. In a preferred method, the expression response is compared by PCR-type assays.

An advantage of in situ hybridization over certain other techniques for the detection of nucleic acids is that it allows an investigator to determine the precise spatial population. In situ hybridization may be used to measure the steady-state level of RNA accumulation. A number of protocols have been devised for in situ hybridization, each with tissue preparation, hybridization and washing conditions.

In situ hybridization also allows for the localization of proteins or mRNA within a tissue or cell. It is understood that one or more of the molecules of the invention, preferably one or more of the nucleic acid molecules or fragments thereof of the invention or one or more of the antibodies of the invention may be utilized to detect the level or pattern of a protein or mRNA thereof by in situ hybridization.

In one aspect of the present invention, an evaluation can be conducted to determine whether a optineurin nucleic acid molecule is present. One or more of the nucleic acid molecules of the present invention are utilized to detect the presence, type, or quantity of the nucleic acid molecule. Generally, such a method comprises: (a) obtaining cell or tissue sample of interest; and (b) selectively detecting the presence or absence, or ascertaining the level of a nucleic acid molecule.

As used herein, the term “presence” refers to when a molecule can be detected using a particular detection methodology. Also as used herein, the term “absence” refers to when a molecule cannot by detected using a particular detection methodology.

The present invention also includes and provides a method for determining a level or pattern of a protein in an animal cell or animal tissue comprising (A) assaying the concentration of the protein in a first sample obtained from the animal cell or animal tissue; (B) assaying the concentration of the protein in a second sample obtained from a reference animal cell or a reference animal tissue with a known level or pattern of the protein; and (C) comparing the assayed concentration of the protein in the first sample to the assayed concentration of the protein in the second sample.

Any method for analyzing proteins can be used to detect or measure levels of a polypeptide. As an illustration, size differences can be detected by Western blots of protein extracts from the two tissues. Other changes, such as expression levels and subcellular localization, can also be detected immunologically, using antibodies to the corresponding protein. The expression pattern of any cell or tissue types can be compared. Such comparison can also occur in a temporal manner. Another comparison can be made between difference developmental states of a tissue or cell sample.

More particularly, in one embodiment, mRNA in a cell or tissue sample can be detected by incubating mRNA molecules with cell or tissue sample extracts of an organism under conditions sufficient to permit nucleic acid hybridization. The detection of double-stranded probe-mRNA hybrid molecules is indicative of the presence of the mRNA; the amount of such hybrid formed is proportional to the amount of mRNA. Thus, such probes may be used to ascertain the level and extent of the mRNA production in an organism's cells or tissues. Such nucleic acid hybridization may be conducted under quantitative conditions (thereby providing a numerical value of the amount of the mRNA present). Alternatively, the assay may be conducted as a qualitative assay that indicates either that the mRNA is present, or that its level exceeds a user set, predefined value.

Alternatively, mRNA may be selectively detected using standard PCR or RT-PCR techniques such as those described herein. In another embodiment, polypeptide molecules of the present invention may be selectively detected using an immunological binding assay, e.g., an in situ binding assay. In this regard, an antibody which selectively binds to an polypeptide of the present invention may be used. Optionally, the antibody may be labeled as described below to aid in detection.

More particularly, polypeptide molecules can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice. The antibody may be produced by any of a number of means well known to those of skill in the art and as described above.

Immunoassays also often use a labeling agent to specifically bind to, and label the complex formed by the antibody and antigen. The labeling agent may itself be one of the moieties comprising the antibody/antigen complex. Thus, the labeling agent may be a labeled polypeptide or a labeled antibody. Alternatively, the labeling agent may be a third moiety, such a secondary antibody, that specifically binds to the antibody/polypeptide complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol., 111:1401-1406 (1973); Akerstrom et al., J. Immunol., 135:2589-2542 (1985)). The labeling agent can be modified with a detectable moiety, such as biotin, to which another molecule can specifically bind, such as streptavidin. A variety of detectable moieties are well known to those skilled in the art. A preferred label is a fluorescent label.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Generally, immunoassays for detecting a polypeptide in a sample may be either competitive or noncompetitive. Noncompetitive immunoassays are assays in which the amount of antigen is directly measured. In one preferred “sandwich” assay, for example, the antibodies can be bound directly to a solid substrate on which they are immobilized. These immobilized antibodies then capture the polypeptide present in the test sample. The polypeptide is thus immobilized, and is then bound by a labeling agent, such as a second antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Western blot (immunoblot) analysis may also used to detect and quantify the presence of polypeptide in the sample. Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev., 5:34-41 (1986)).

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Thus, in one aspect of the present invention, provided are methods for diagnosing glaucoma in a sample obtained from a cell or a bodily fluid by detecting a polymorphism in a promoter region of the optineurin gene, comprising the steps of: (A) incubating under conditions permitting nucleic acid hybridization, a marker nucleic acid molecule, the marker nucleic acid molecule having a nucleic acid sequence that specifically hybridizes to a sequence selected from the group consisting of SEQ ID NO: 1 and a complement thereof, and a complementary nucleic acid molecule obtained from a sample, wherein nucleic acid hybridization between the marker nucleic acid molecule and the complementary nucleic acid molecule permits the detection of said polymorphism; (B) permitting hybridization between the marker nucleic acid molecule and the complementary nucleic acid molecule; and (C) detecting the presence of the polymorphism, wherein the detection of the polymorphism is diagnostic of glaucoma.

Also provided by the present invention are methods for prognosing glaucoma in a sample obtained from a cell or a bodily fluid by detecting a polymorphism in a promoter region of the optineurin gene, comprising the steps of: (A) incubating under conditions permitting nucleic acid hybridization, a marker nucleic acid molecule, the marker nucleic acid molecule having a nucleic acid sequence that specifically hybridizes to a sequence selected from the group consisting of SEQ ID NO: 1 and complement thereof, and a complementary nucleic acid molecule obtained from a sample, where nucleic acid hybridization between the marker nucleic acid molecule and the complementary nucleic acid molecule permits the detection of the polymorphism; (B) permitting hybridization between the marker nucleic acid molecule and the complementary nucleic acid molecule; and (C) detecting the presence of the polymorphism, where the detection of the polymorphism is prognostic of glaucoma.

Further provided by the present invention are methods for diagnosing or prognosing glaucoma in a sample obtained from a cell or a bodily fluid by detecting a polymorphism in a promoter region of the optineurin gene, comprising the steps of: (A) incubating under conditions permitting nucleic acid hybridization, a marker nucleic acid molecule, the marker nucleic acid molecule having a nucleic acid sequence that specifically hybridizes to a optineurin promoter sequence or its complement, and a complementary nucleic acid molecule obtained from a sample, where nucleic acid hybridization between the marker nucleic acid molecule and the complementary nucleic acid molecule permits the detection of the polymorphism; (B) permitting hybridization between the marker nucleic acid molecule and the complementary nucleic acid molecule; and (C) detecting the presence of the polymorphism, where the detection of the polymorphism is diagnostic or prognostic of glaucoma.

The methods of the present invention may be used to detect a single nucleotide polymorphism, and may further comprise a second marker nucleic acid molecule.

The present invention further provides methods for detecting the presence or absence of a SNP sequence variation in a sample containing DNA, comprising contacting a labeled nucleic acid capable of detecting a single nucleotide polymorphism selected from table 1 with the DNA of the sample under hybridization conditions and determining the presence of hybrid nucleic acid molecules comprising the labeled nucleic acid.

The cell or bodily fluid may comprise human trabecular meshwork cells, or may be selected from the group consisting of glaucomatous cell extract, fluid from the anterior chamber of the eye, blood, lymph, and serum. The methods may further comprise amplifying the complementary nucleic acid molecule obtained from a sample using a nucleic acid amplification method, where the nucleic acid amplification method is selected from the group consisting of polymerase chain amplification, ligase chain reaction, oligonucleotide ligation assay, thermal amplification, and transcription base amplification.

The diagnostic and prognostic methods described herein can, for example without limitation, utilize one or more of the detection methods described herein, including but not limited to northern blot analysis, standard PCR, reverse transcription-polymerase chain reaction (RT-PCR), in situ hybridization, immunoprecipitatioon, Western blot hybridization, or immunohistochemistry.

In one aspect, the method comprises in situ hybridization with a nucleic acid molecule of the present invention as a probe. This method comprises contacting the labeled hybridization probe with a sample of a given type of tissue potentially containing glaucomatous or pre-glaucomatous cells as well as normal cells, and determining whether the probe labels some cells of the given tissue type to a degree significantly different (e.g., by at least a factor of two, or at least a factor of five, or at least a factor of twenty, or at least a factor of fifty) than the degree to which it labels other cells of the same tissue type.

Alternatively, the above diagnostic assays may be carried out using antibodies which selectively detect a polypeptide of the present invention. Accordingly, in one embodiment, the assay includes contacting the proteins of the test cell with an antibody specific for a polypeptide of the present invention and determining the approximate amount of immunocomplex formation. Such a complex can be detected by an assay for example without limitation an immunohistochemical assay, dot-blot assay, and an ELISA assay.

Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

G. Modulator Screening Assays

Another aspect of the invention is directed to the identification of agents capable of modulating one or more optineurin molecules. Such agents are herein referred to as “modulators” or “modulating compounds”. In this regard, the invention provides assays for determining compounds that modulate the function and/or expression of one or more optineurin molecules.

“Inhibitors,” “activators,” and “modulators” of optineurin molecules are used interchangeably to refer to inhibitory, activating, or modulating molecules which can be identified using in vitro and in vivo assays for optineurin activity and/or expression, e.g., ligands, agonists, antagonists, and their homologs and mimetics.

Suitable modulators include, but are not limited to, hydroxamic acids, diclofenac, MMP inhibitors, macrocyclic anti-succinate hydroxamate derivatives, anti-angiogenics, tetracyclines, steroid inactivators of metalloproteinase translation, DNA binding (minor groove) compounds, peptide-like agents such as TIMPs, N-carboxyalkyl peptides, polyamines and glycosaminoglycans, non-steroidal anti-inflammatory drugs (NSAIDs), corticosteroids, immunosuppressive agents, antibiotics, receptor antagonists, RNA aptamers, and antibodies.

Anti-angiogenics comprise a class of compounds including growth factors, cytokines and peptides, which share characteristics such as the ability to inhibit angiogenesis, endothelial cell proliferation, migration, tube formation and neovascularization. Preferred anti-angiogenics include endostatin and active collagen fragment derivatives, such as arresten (a 26 kDa NC1 domain of the alpha 1 chain of type IV collagen), thrombospondin, interleukin-12, angiostatin and active fragments and derivatives of plasminogen. See Colorado et al., Cancer Research 60(9):2520-26 (2000); Sunamura et al., Pancreas 20(3):227-33 (2000); Griscelli et al., Proceedings of the National Academy of Sciences U.S.A., 95(11):6367-72 (1998). Other preferred anti-angiogenics are growth factors such as basic fibroblast growth factor (bFGF), which may be used alone or in combination with other anti-angiogenics such as all-trans retinoic acid to stimulate native MMP inhibitors such as tissue inhibitor of metalloproteinases-1 (TIMP-1) protein. See Bigg et al., European Journal of Biochemistry 267(13):4150-56 (2000).

Hydroxamic acid-based modulators are described in U.S. Pat. No. 5,240,958, and preferably have the general formula:

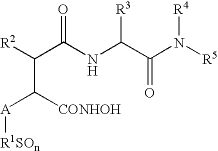

where R¹ represents thienyl; R² represents a hydrogen atom or a C₁-C₆ alkyl, C₁-C₆ alkenyl, phenyl(C₁-C₆) alkyl, cycloalkyl(C₁-C₆)alkyl or cycloalkenyl(C₁-C₆)alkyl group; R³ represents an amino acid side chain or a C₁-C₆ alkyl, benzyl, (C₁-C₆alkoxyl)benzyl or benzyloxy(C₁-C₆ alkyl) or benzyloxy benzyl group; R⁴ represents a hydrogen atom or a C₁-C₆ alkyl group; R⁵ represents a hydrogen atom or a methyl group; n is an integer having the value 0, 1 or 2; and A represents a C₁-C₆ hydrocarbon chain, optionally substituted with one or more C₁-C₆ alkyl, phenyl or substituted phenyl groups; or a salt thereof.

Other hydroxamic acid-based modulators include phosphinamide-based hydroxamic acids, peptidyl hydroxamic acids including p-NH₂-Bz-Gly-Pro-D-Leu-D-Ala-NHOH (FN-439), hydroxamic acids with a quaternary-hydroxy group, and succinate-derived hydroxamic acids related to batimastat. See, e.g., Pikul et al., Journal of Medical Chemistry 42(l):87-94 (1999); Odake et al., Biochem Biophys Res Commun 199(3):1442-46 (1994); Jacobson et al., Bioorganic Medical Chemistry Letters 8(7):837-42 (1998); Steimnan et al., Bioorganic Medical Chemistry Letters 8(16):2087-92 (1998). Macrocyclic anti-succinate hydroxamate derivatives can also be effective modulators. See Cherney et al., Bioorganic Medical Chemistry Letters 9(9):1279-84 (1999). Batimastat, also known as BB-94, is a relatively insoluble chemical having the chemical name [2-R-[1(S*),2R*,3S*]]-N⁴-hydroxy-N¹-[2-(methylamino)-2-oxo-1-(phenylmethyl)ethyl]-2-(2-methylpropyl)-3-[(2-thienylthio)methyl] butanediamide or (2S,-3R)-5-methyl-3-[[(αS)-α-(methylcarbamoyl)phenethyl]carbamoyl]-2-[(2-thienylthio)methyl]hexanohydroxamic acid, and the formula:

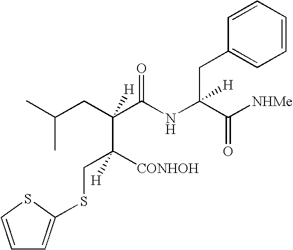

Other preferred modulators include the tetracyclines, especially minocycline, doxycycline, and COL-3, and steroid inactivators of metalloproteinase translation, such as dexamethasone. See Fife et al., Cancer Letters 153(1-2):75-8 (2000); Gilbertson-Beadling et al., Cancer Chemother. Pharmacol. 36(5):418-24 (1995); Greenwald et al., Journal of Rheumatology 19(6):927-38 (1992); Shapiro et al., Journal of Immunology 146(8):2724-29 (1991). A further group of modulators includes DNA binding (minor groove) compounds such as distamycin A and its sulphonic derivatives PNU145156E and PNU153429, anthramycin, pyrrolo[2,1-c][1,4]benzodiazepine (PBD) and its methyl esters, and other polypyrrole minor groove binders. See, e.g., Baraldi et al., Journal of Medical Chemistry 42(25):5131-41 (1999); Possati et al., Clin. Exp. Metastasis 17(7):575-82 (1999).

The peptide-like modulators comprise a varied class of compounds that includes peptides, peptide mimetics, pseudopeptides, polyamines, and glycosaminoglycans. Tissue inhibitors of metalloproteinases (TIMPs) are peptides and polypeptides that inhibit the action of metalloproteinases and that share structural characteristics such as intrachain disulfide bonds. Preferred TIMPs include recombinant and isolated forms of natural TIMPs, including TIMP-1 (a 28.5 kDa polypeptide), TIMP-2 (a 21 kDa polypeptide), and TIMP-3 (a 24-25 kDa polypeptide), and fragments thereof that retain inhibitory function. See G. Murphy et al., Biochemistry 30(33):8097-102 (1991); A. N. Murphy et al., Journal of Cell Physiology 157(2):351-58 (1993); Kishnani et al., Matrix Biology 14(6):479-88 (1995).

N-carboxyalkyl peptides are a class of peptides that include CH₃CH₂CH₂(R,S)CH(COOH)-NH-Leu-Phe-Ala-NH₂, N-[D,L-2-isobutyl-3(N′-hydroxycarbonylamido)-propanoyl]-O-methyl-L-tyrosine methylamide, and HSCH₂CH[CH₂CH(CH₃)₂]CO-Phe-Ala-NH₂ (SIMP). See Fini et al., Invest. Ophthalmol. Vis. Sci. 32(11):2997-3001 (1991); Stack et al., Arch. Biochem. Biophys. 287(2):240-49 (1991); Wentworth et al., Invest. Ophthalmol. Vis. Sci. 33(7):2174-79 (1992). Other peptide-like modulators include polyamines such as alpha-difluoromethylomithine, and glycosaminoglycans such as combretastatin and heparin. See Wallon et al., Mol. Carcinog. 11(3):138-44 (1994); Dark et al., Cancer Research 57 (10):1829-34 (1997); Lyons-Giordano et al., Exp. Cell Research 186(1):39-46 (1990).

Sulfur-based modulators such as sulfonanilides and sulfonamides may also be used as modulators. Preferred sulfur-based modulators include sulfonanilide nonsteroidal anti-inflammatory drugs (NSAIDs) such as nimesulide, acyclic sulfonamides, and malonyl alpha-mercaptoketones and alpha-mercaptoalcohols. See, e.g., Bevilacqua et al., Drugs 46 Suppl. 1:40-47 (1993); Hanessian et al., Bioorganic Medical Chemistry Letters 9(12):1691-96 (1999); Campbell et al., Bioorganic Medical Chemistry Letters 8(10):1157-62 (1998).

Another class of modulators includes compounds that antagonize receptors involved in posterior segment ophthalmic disorders, e.g., vascular endothelial growth factor (VEGF) receptors. VEGF antagonists include peptides that inhibit the binding of VEGF to its receptors, such as short disulfide-constrained peptides. See Fairbrother et al., Biochemistry 37(51):17754-64 (1998); Binetruy-Tournaire et al., EMBO J. 19(7): 1525-33 (2000). VEGF antagonists inhibit the outgrowth of blood vessels by inhibiting the ability of VEGF to contact its receptors. This mechanism of anti-angiogenesis operates differently than the mechanism caused by the stimulation of growth factors such as bFGF, which act to inhibit angiogenesis by stimulating native inhibitors of proteases. Other VEGF antagonists may be derived from asymmetric variants of VEGF itself. See, e.g., Siemester et al., Proceedings of the National Academy of Sciences U.S.A. 95:4625-29 (1998). Other useful modulators are RNA aptamers, which may be designed to antagonize VEGF or the closely related platelet-derived growth factor (PDGF), and may be administered coupled to polyethylene glycol or lipids. See, e.g., Floege et al., American Journal of Pathology 154(1):169-79 (1999); Ostendorf et al., J. Clin. Invest. 104(7):913-23 (1999); Willis et al., Bioconjug. Chem. 9(5):573-82 (1998).

Modulator screening may be performed by adding a putative modulator test compound to a tissue or cell sample, and monitoring the effect of the test compound on the function and/or expression of optineurin. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds. Differences between treated and untreated cells indicates effects attributable to the test compound.

The invention thus also encompasses methods of screening for agents which inhibit promotion or expression of an optineurin molecule in vitro, comprising exposing a cell or tissue in which the optineurin molecule is detectable in cultured cells to an agent in order to determine whether the agent is capable of inhibiting production of the optineurin molecule; and determining the level of optineurin molecule in the exposed cells or tissue, where a decrease in the level of the optineurin molecule after exposure of the cell line to the agent is indicative of inhibition of the optineurin molecule.

Alternatively, the screening method may include in vitro screening of a cell or tissue in which an optineurin molecule is detectable in cultured cells to an agent suspected of inhibiting production of the optineurin molecule; and determining the level of the optineurin molecule in the cells or tissue, where a decrease in the level of optineurin molecule after exposure of the cells or tissue to the agent is indicative of inhibition of optineurin molecule production.

The invention also encompasses in vivo methods of screening for agents which inhibit expression of the optineurin molecules, comprising exposing a mammal having glaucomatous cells in which an optineurin molecule is detectable to an agent suspected of inhibiting production of the optineurin molecule; and determining the level of optineurin molecule in glaucomatous cells of the exposed mammal. A decrease in the level of optineurin molecule after exposure of the mammal to the agent is indicative of inhibition of marker nucleic acid expression.

Accordingly, the invention provides a method comprising incubating a cell expressing the optineurin molecule with a test compound and measuring the optineurin molecule level. The invention further provides a method for quantitatively determining the level of expression of the optineurin molecule in a cell population, and a method for determining whether an agent is capable of increasing or decreasing the level of expression of the optineurin molecule in a cell population.

The invention also encompasses a method for determining whether an agent is capable of increasing or decreasing the level of expression of the optineurin molecule in a cell population comprises the steps of (a) preparing cell extracts from control and agent-treated cell populations, (b) isolating the optineurin molecule from the cell extracts, (c) quantifying (e.g., in parallel) the amount of an immunocomplex formed between the optineurin molecule and an antibody specific to said optineurin molecule.

mRNA levels can be determined by Northern blot hybridization. mRNA levels can also be determined by methods involving PCR. Other sensitive methods for measuring mRNA, which can be used in high throughput assays, e.g., a method using a DELFIA endpoint detection and quantification method, are described, e.g., in Webb and Hurskainen Journal of Biomolecular Screening 1:119 (1996). Optineurin molecule levels can be determined by immunoprecipitations or immunohistochemistry using an antibody that specifically recognizes the protein product encoded by the nucleic acid molecules.

Agents that are identified as active in the drug screening assay are candidates to be tested for their capacity to block or promote glaucoma.

H. In vivo Methods and Therapeutic Applications

The pharmaceutical compositions of the present invention, including antisense formulations, may be therapeutically used in clinical settings to affect glaucoma. As described above, the optineurin promoter contains response elements which allow for the regulation of optineurin expression, and affecting the activity of a response element can at least partially inhibit or block glaucoma induced in cells by optineurin expression.

As used herein, “at least partially inhibiting” refers to the reduction of a particular event, for example without limitation, the function and/or expression of optineurin polypeptides. In a preferred embodiment, to determine whether a particular event is “at least partially inhibited”, the sample of interest subject to a particular method or agent is compared with similar sample of interest not subjected to the particular method or agent. In one embodiment, an inhibition of a particular event is statistically significant. In a particularly preferred embodiment, a particular event is inhibited in a sample of interest by 25%, 50%, 60%, 70%, 75%, 80%, 85%, 90 %, 95% or 100%, as compared to a similar sample of interest not subjected to the particular event. More particularly, as used herein, “blocking” refers to inhibition of a particular event in a sample of interest by greater than 90%, as compared to a similar sample of interest not subject to the particular event.

Accordingly, one aspect of the present invention is directed to the use of optineurin nucleic acid molecules to at least partially inhibit, alter, or retard the development of glaucoma mediated by optineurin. Another aspect of the present invention is directed to the use of antisense optineurin nucleic acid molecules as therapeutic molecules to at least partially inhibit or block (knockdown/knockout) expression of natural optineurin. A further aspect of the present invention is directed to the use of antisense optineurin nucleic acid molecules as therapeutic molecules to at least partially enhance or increase the expression of natural optineurin. The consequence of altering the expression of natural optineurin would be to affect the onset, progression, or development of glaucoma. A particular application would be for the treatment of glaucomas, particularly those where optineurin is expressed at non-normal levels.

In yet another embodiment, a method for at least partially inhibiting the production of an optineurin polypeptide in a cell is provided comprising: (a) providing an isolated nucleic acid molecule comprising at least 10 consecutive nucleotides of the complement of SEQ ID NOs: 3 through 463; (b) introducing the nucleic acid molecule into the cell; and (c) maintaining the cell under conditions permitting the binding of the nucleic acid sequence to optineurin mRNA.

I. Markers

Another subset of the nucleic acid molecules of the invention includes nucleic acid molecules that are markers. As used herein, a “marker” is an indicator for the presence of at least one phenotype or polymorphism, such as single nucleotide polymorphisms (SNPs), cleavable amplified polymorphic sequences (CAPs), amplified fragment length polymorphisms (AFLPs), restriction fragment length polymorphisms (RFLPs), simple sequence repeats (SSRs), or random amplified polymorphic DNA (RAPDs). The markers can be used in a number of ways in the field of molecular genetics.

In one embodiment of the present invention, the marker specifically hybridizes to a nucleic acid molecule having a nucleic acid sequence selected from the group of SEQ ID NOs: 1-463, fragments thereof and complements of either. In a preferred embodiment, the marker is capable of detecting a SNP set forth in Table 2. In another preferred embodiment, the marker is capable of acting as a PCR primer to amplify a region set forth in Table 1. Such markers include nucleic acid molecules SEQ ID NOs: 1-463 or complements thereof or fragments of either that can act as markers and other nucleic acid molecules of the present invention that can act as markers.

Genetic markers of the invention include “dominant” or “codominant” markers. “Codominant markers” reveal the presence of two or more alleles (two per diploid individual) at a locus. “Dominant markers” reveal the presence of only a single allele per locus. The presence of the dominant marker phenotype (e.g., a band of DNA) is an indication that one allele is in either the homozygous or heterozygous condition. The absence of the dominant marker phenotype (e.g., absence of a DNA band) is merely evidence that “some other” undefined allele is present. In the case of populations where individuals are predominantly homozygous and loci are predominately dimorphic, dominant and codominant markers can be equally valuable. As populations become more heterozygous and multi-allelic, codominant markers often become more informative of the genotype than dominant markers. Marker molecules can be, for example, capable of detecting polymorphisms such as single nucleotide polymorphisms (SNPs).

The genomes of animals and plants naturally undergo spontaneous mutation in the course of their continuing evolution. A “polymorphism” is a variation or difference in the sequence of the gene or its flanking regions that arises in some of the members of a species. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist and the polymorphism is thus said to be di-allelic. In other cases, the species' population may contain multiple alleles and the polymorphism is termed tri-allelic, etc. A single gene may have multiple different unrelated polymorphisms. For example, it may have a di-allelic polymorphism at one site and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRs) that include tandem di- or tri-nucleotide repeated motifs of nucleotides. Polymorphisms characterized by such tandem repeats are referred to as “variable number tandem repeat” (VNTR) polymorphisms. VNTRs have been used in identity analysis (EP 370719; U.S. Pat. Nos. 5,075,217 and 5,175,082; WO 91/14003).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s) and more preferably within 100 kb of the polymorphism(s) and most preferably within 10 kb of the polymorphism(s) can be employed. Alternatively, marker nucleic acid molecules comprising a nucleotide sequence of a polynucleotide located within 25 cM of the polymorphism(s) and more preferably within 15 cM of the polymorphism(s) and most preferably within 5 cM of the polymorphism(s) can be employed.

The identification of a polymorphism can be determined in a variety of ways. By correlating the presence or absence of it in an organism with the presence or absence of a phenotype, it is possible to predict the phenotype of that organism. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, organisms that possess a variant sequence can be distinguished from those having the original sequence by restriction fragment analysis. Polymorphisms that can be identified in this manner are termed “restriction fragment length polymorphisms” (RFLPs) (UK Patent Application 2135774; WO 90/13668; WO 90/11369).

Polymorphisms can also be identified by Single Strand Conformation Polymorphism (SSCP) analysis, random amplified polymorphic DNA (RAPD), and cleaveable amplified polymorphic sequences (CAPS). See, e.g., Lee et al., Anal. Biochem. 205:289-293 (1992); Sarkar et al., Genoomics 13:441-443 (1992); Williams et al., Nucl. Acids Res. 18:6531-6535 (1990); and Lyamichev et al., Science 260:778-783 (1993). It is understood that one or more of the nucleic acids of the invention, may be utilized as markers or probes to detect polymorphisms by SSCP, RAPD or CAPS analysis.

Polymorphisms may also be found using a DNA fingerprinting technique called amplified fragment length polymorphism (AFLP), which is based on the selective PCR amplification of restriction fragments from a total digest of genomic DNA to profile that DNA. Vos et al., Nucleic Acids Res. 23:4407-4414 (1995). This method allows for the specific co-amplification of high numbers of restriction fragments, which can be visualized by PCR without knowledge of the nucleic acid sequence. It is understood that one or more of the nucleic acids of the invention may be utilized as markers or probes to detect polymorphisms by AFLP analysis or for fingerprinting RNA.

Single Nucleotide Polymorphisms (SNPs) generally occur at greater frequency than other polymorphic markers and are spaced with a greater uniformity throughout a genome than other reported forms of polymorphism. The greater frequency and uniformity of SNPs means that there is greater probability that such a polymorphism will be found near or in a genetic locus of interest than would be the case for other polymorphisms. SNPs are located in protein-coding regions and noncoding regions of a genome. Some of these SNPs may result in defective or variant protein expression (e.g., as a result of mutations or defective splicing). Analysis (genotyping) of characterized SNPs can require only a plus/minus assay rather than a lengthy measurement, permitting easier automation.

SNPs can be characterized using any of a variety of methods. Such methods include the direct or indirect sequencing of the site, the use of restriction enzymes, enzymatic and chemical mismatch assays, allele-specific PCR, ligase chain reaction, single-strand conformation polymorphism analysis, single base primer extension (U.S. Pat. Nos. 6,004,744 and 5,888,819), solid-phase ELISA-based oligonucleotide ligation assays, dideoxy fingerprinting, oligonucleotide fluorescence-quenching assays, 5′-nuclease allele-specific hybridization TaqMan™ assay, template-directed dye-terminator incorporation (TDI) assay (Chen and Kwok, Nucl. Acids Res. 25:347-353, 1997), allele-specific molecular beacon assay (Tyagi et al., Nature Biotech. 16: 49-53, 1998), PinPoint assay (Haff and Smirnov, Genome Res. 7: 378-388, 1997), dCAPS analysis (Neff et al., Plant J. 14:387-392, 1998), pyrosequencing (Ronaghi et al., Analytical Biochemistry 267:65-71, 1999; WO 98/13523; WO 98/28440; and www.pyrosequencing.com), using mass spectrometry, e.g. the Masscode™ system (WO 99/05319; WO 98/26095; WO 98/12355; WO 97/33000; WO 97/27331; www.rapigene.com; and U.S. Pat. No. 5,965,363), invasive cleavage of oligonucleotide probes, and using high density oligonucleotide arrays (Hacia et al., Nature Genetics 22:164-167; www.affymetrix.com).

Polymorphisms may also be detected using allele-specific oligonucleotides (ASO), which, can be for example, used in combination with hybridization based technology including Southern, northern, and dot blot hybridizations, reverse dot blot hybridizations and hybridizations performed on microarray and related technology.

The stringency of hybridization for polymorphism detection is highly dependent upon a variety of factors, including length of the allele-specific oligonucleotide, sequence composition, degree of complementarity (i.e. presence or absence of base mismatches), concentration of salts and other factors such as formamide, and temperature. These factors are important both during the hybridization itself and during subsequent washes performed to remove target polynucleotide that is not specifically hybridized. In practice, the conditions of the final, most stringent wash are most critical. In addition, the amount of target polynucleotide that is able to hybridize to the allele-specific oligonucleotide is also governed by such factors as the concentration of both the ASO and the target polynucleotide, the presence and concentration of factors that act to “tie up” water molecules, so as to effectively concentrate the reagents (e.g., PEG, dextran, dextran sulfate, etc.), whether the nucleic acids are immobilized or in solution, and the duration of hybridization and washing steps.

Hybridizations are preferably performed below the melting temperature (Tₘ) of the ASO. The closer the hybridization and/or washing step is to the Tₘ, the higher the stringency. Tₘ for an oligonucleotide may be approximated, for example, according to the following formula:

Tₘ=81.5+16.6×(log10[Na+])+0.41×(%G+C)−675/n;

where [Na+] is the molar salt concentration of Na+ or any other suitable cation and n=number of bases in the oligonucleotide. Other formulas for approximating Tₘ are available and are known to those of ordinary skill in the art.

Stringency is preferably adjusted so as to allow a given ASO to differentially hybridize to a target polynucleotide of the correct allele and a target polynucleotide of the incorrect allele. Preferably, there will be at least a two-fold differential between the signal produced by the ASO hybridizing to a target polynucleotide of the correct allele and the level of the signal produced by the ASO cross-hybridizing to a target polynucleotide of the incorrect allele (e.g., an ASO specific for a mutant allele cross-hybridizing to a wild-type allele). In more preferred embodiments of the present invention, there is at least a five-fold signal differential. In highly preferred embodiments of the present invention, there is at least an order of magnitude signal differential between the ASO hybridizing to a target polynucleotide of the correct allele and the level of the signal produced by the ASO cross-hybridizing to a target polynucleotide of the incorrect allele. While certain methods for detecting polymorphisms are described herein, other detection methodologies may be utilized.

The identification of a polymorphism in the optineurin gene, or flanking sequences up to about 7,500 bases from either end of the coding region, can be determined in a variety of ways. By correlating the presence or absence of glaucoma in an individual with the presence or absence of a polymorphism in the optineurin gene or its flanking regions, it is possible to diagnose the predisposition (prognosis) of an asymptomatic patient to glaucoma or related diseases.

In accordance with this embodiment of the invention, a sample DNA is obtained from a patient. In a preferred embodiment, the DNA sample is obtained from the patient's blood, however, any source of DNA may be used. The DNA is subjected to restriction endonuclease digestion using the optineurin promoter or fragments thereof as a probe in accordance with the above-described RFLP methods. By comparing the RFLP pattern of the optineurin gene obtained from normal and glaucomatous patients, one can determine a patient's predisposition (prognosis) to glaucoma. The polymorphism obtained in this approach can then be cloned to identify the mutation at the regulatory region of the gene which affects its expression level. Changes involving promoter interactions with other regulatory proteins can be identified by, for example, gel shift assays using HTM cell extracts, fluid from the anterior chamber of the eye, serum, etc.

Several different classes of polymorphisms may be identified through such methods. Examples of such classes include polymorphisms in non-translated optineurin gene sequences, including the promoter or other regulatory regions, and polymorphisms in genes whose products interact with optineurin regulatory sequences.

### EXAMPLE 1

### IDENTIFICATION OF SNPs IN THE OPTINEURIN PROMOTER

To identify novel SNPs in the promoter region up to 5 kb upstream of the transcription initiation site, genomic DNA from 23 individuals is sequenced. The individuals include 7 normal subjects, 8 POAG patients with increased intra-ocular tension, and 8 NTG patients. DNA from these individuals is sequenced over 5000 nucleotides. Between 3 and 5 amplicons are required to sequence the optineurin promoter region over 5 kb, which number depends on the number and nature of repetitive sequences and GC richness of the promoter. Each amplicon is sequenced on one or both strands to detect presence of the SNPs.

Amplifications are carried out using a “hot-start” procedure. Samples are processed through 35 cycles of denaturation (95° C. for 30 s) and annealing (55-60° C. for 30 s), followed by one last step of elongation (72° C. for 50 s). PCR products are diluted in 5 volumes of Qiagen PB buffer (Qiagen, Valencia, Calif.), transferred onto a Whatman GF/C filter plate (Whatman Group, Ann Arbor Mich.), washed two times with an 80% ethanol 20 mM Tris pH 7.5, and eluted in 50 microliters of water. Samples are quantified using the PicoGreen reagent protocol (Molecular Probes, Eugene, Oreg.). A second PCR is performed on an Applied Biosystem Gene Amp PCR System 9700 (96 wells) or 9700 Viper (384 wells)(Applied Biosystem, Foster City, Calif.) to incorporate the sequencing dyes using a protocol of 25 cycles of denaturation (95° C. for 10 s) and annealing (55° C. for 5 s), followed by one last step of elongation (59° C. for 2 min). PCR products are purified by the ABI ethanol-EDTA precipitation protocol, collected in a Beckman-Couter Allegra 6R centrifuge (Beckman-Coulter, Inc., Fullerton, Calif.) and resuspended in a 50% HiDi-formamide solution. Samples are run on an Applied Biosystems 3700 DNA Analyser automated sequencer.

Sequence data is analyzed with the Staden preGap4 and Gap4 programs Griffen, Computer Analysis of Sequence, Part 1 (Humana Press, 1994). Sequencing data and all patients' information is stored in a 4D database on a MacIntosh G4. Data is transferred from the 4D database to SUN computers using CAP AppleShare server software. Several SNPs are identified in the promoter region and their allelic frequencies in patients and controls are calculated (Table 4). Genotypic frequencies may also be calculated for identified SNPs (Table 5).

TABLE 4
SNPs and Allelic Frequencies

| | | | Allelic Frequency of Variant | | |
|---|---|---|---|---|---|
| | | Number of | POAG | NTG | Normal |
| Location^{\} | CN* | Subjects | Patients | Patients | (control) |
| 391 | a/g | 27 | 3/10 (30%) | 5/8 (62.5%) | 3/9 (33%) |
| 709 | g/a | 29 | 3/10 (30.0%) | 1/10 (10.0%) | 0/8 (0%) |
| 887 | t/a | 29 | 1/11 (9.1%) | 0/10 (0%) | 0/8 (0%) |

^{\}Location in SEQ ID NO:1; *Characteristic Nucleotides

TABLE 5
Genotypic Frequencies for an Optineurin Promoter SNP

| SNP Location^{†} | | Genotypic Frequencies | | |
|---|---|---|---|---|
| & CN* | Subject Group | aa | ag | gg |
| 2606 | POAG Patients | 1 (9.1) | 9 (81.8%) | 1 (9.1) |
| a/g | (n = 11) | | | |
| | NTG Patients | 2 (18.2%) | 7 (63.6%) | 2 (18.2%) |
| | (n = 11) | | | |
| | Normal (control) | 1 (24.3%) | 5 (71.4%) | 1 (24.3%) |
| | (n = 7) | | | |

^{\}Location in SEQ ID NO:1; *Characteristic Nucleotides

### EXAMPLE 2

### VECTOR CONSTRUCTION

Expression vectors can be constructed for efficient expression of an optineurin promoter construct (e.g., the optineurin promoter operably linked to a heterologous nucleic acid, etc.) in mammalian cell lines. These expression vectors generally include the optineurin promoter operably linked to a nucleic acid sequence. The vectors can also be designed to confer antibiotic or toxin resistance through expression of resistance genes under control of a second promoter. Illustrative vectors include pcDNA3.1 and pMEP4 (Invitrogen, Carlsbad, Calif.).

For example, the CMV2 promoter is deleted from mammalian vector pTracer CMV2 (Invitrogen) and replaced with a nucleic acid molecule having SEQ ID NO: 1 linked in an manner that facilitates expression of the green florescent protein (pTrOp). Chinese hamster ovary cells (CHO) are then transfected with either pTracer CMV2 or pTrOp using the method set forth in Cameri et al., Nature Biotechnology 14: 315-319 (1996). Levels of green fluorescent protein are measured using the method set forth in Cameri et al., Nature Biotechnology 14: 315-319 (1996).

### EXAMPLE 3

### MODULATOR SCREENING

The transfected cell lines described in Example 2 containing either pTracer CMV2 or pTrOp are grown in a cell medium described by Miller et al. J. Biol. Chem. 274 20465-20472 (1999) supplemented by a test compound. The level of green fluorescent protein is measured using the method set forth in Cameri et al., Nature Biotechnology 14: 315-319 (1996) across a range of test compounds and effective concentrations in the CHO cell lines containing either pTracer CMV2 or pTrOp.

All references, publications, and patents cited herein are specifically incorporated by reference in a manner consistent with this disclosure. Reagents and compositions (e.g., nucleic acid molecule, amino acid molecules, vectors, host cells, antibodies, etc.) related to optineurin can be made using methodologies known to those of skill in the art or may be obtained from commercial suppliers.

What is claimed is:

1. An isolated nucleic acid molecule that comprises at least 20 consecutive nucleotides but not more than 1500 consecutive nucleotides of the sequence of SEQ ID NO: 1.

2. An isolated nucleic acid molecule comprising a promoter which comprises at least 20 consecutive nucleotides but not more than 1500 consecutive nucleotides of the sequence of SEQ ID NO: 1, said promoter being operably linked to a heterologous nucleic acid sequence.

3. The isolated nucleic acid molecule according to claim 2, where said heterologous nucleic acid sequence is capable of being expressed in ocular tissue.

4. The isolated nucleic acid molecule according to claim 2, where said heterologous nucleic acid sequence is capable of being expressed in optic nerve cells.

5. The isolated nucleic acid molecule according to claim 2, where said heterologous nucleic acid sequence is capable of being expressed in retinal cells.

6. The isolated nucleic acid molecule according to claim 2, where said heterologous nucleic acid sequence is capable of being expressed in trabecular meshwork cells.

7. The isolated nucleic acid molecule according to claim 2, where said heterologous nucleic acid sequence is selected from the group consisting of a coding sequence, a toxin, and a reporter gene.

8. The isolated nucleic acid molecule according to claim 7, wherein the reporter gene is selected from the group consisting of green fluorescent protein and luciferase.

9. The isolated nucleic acid molecule according to claim 2, where said heterologous nucleic acid sequence is capable of being transcribed as an antisense RNA.

10. The isolated nucleic acid molecule according to claim 9, wherein said antisense RNA is capable of binding to a nucleic acid molecule having the nucleotide sequence of SEQ ID NO: 1 or complements thereof under physiological conditions.

11. The isolated nucleic acid molecule according to claim 10, wherein said antisense RNA is capable of binding to a nucleic acid molecule having a nucleotide sequence selected from the group consisting of SEQ ID NOs: 3 through 463 and complements thereof under physiological conditions.

12. A nucleic acid molecule capable of detecting a single nucleotide polymorphism selected from table 1.

13. The nucleic acid molecule according to claim 12, wherein the nucleic acid molecule is capable of detecting a single nucleotide polymorphism selected from table 4.

14. The nucleic acid molecule according to claim 12, wherein said nucleic acid molecule is capable of detecting a guanine.

15. The nucleic acid molecule according to claim 12, wherein said nucleic acid molecule is capable of detecting a cytosine.

16. The nucleic acid molecule according to claim 12, wherein said nucleic acid molecule is capable of detecting a thymine.

17. The nucleic acid molecule according to claim 12, wherein said nucleic acid molecule is capable of detecting an adenine.

18. The nucleic acid molecule according to claim 12, wherein said nucleic acid molecule does not specifically hybridize to a nucleic acid molecule consisting of SEQ ID NO: 1.

19. A nucleic acid molecule capable of detecting a single nucleotide polymorphism in an optineurin promoter by specifically detecting said single nucleotide polymorphism in said optineurin promoter, wherein said nucleic acid molecule does not specifically hybridize to a nucleic acid molecule consisting of SEQ ID NO: 1.

20. A host cell comprising a nucleic acid molecule comprising a promoter which comprises at least 20 consecutive nucleotides but not more than 1500 consecutive nucleotides of the sequence of SEQ ID NO: 1, said promoter being operably linked to a heterologous nucleic acid sequence.

21. The host cell of claim 20, wherein said host cell is selected from the group consisting of a non-human mammalian cell, a bacterial cell, and an isolated human cell.

22. A method for diagnosing glaucoma in a sample obtained from a cell or a bodily fluid by detecting a polymorphism in a promoter region of the optineurin gene, comprising the steps of:
(A) incubating under conditions permitting nucleic acid hybridization, a marker nucleic acid molecule, said marker nucleic acid molecule having a nucleic acid sequence that specifically hybridizes to a sequence selected from the group consisting of SEQ ID NO: 1 and a complement thereof, and a complementary nucleic acid molecule obtained from a sample, wherein nucleic acid hybridization between said marker nucleic acid molecule and said complementary nucleic acid molecule permits the detection of said polymorphism;
(B) permitting hybridization between said marker nucleic acid molecule and said complementary nucleic acid molecule; and
(C) detecting the presence of said polymorphism, wherein the detection of said polymorphism is diagnostic of glaucoma.

23. The method for diagnosing glaucoma of claim 22, wherein said polymorphism is a single nucleotide polymorphism.

24. The method for diagnosing glaucoma of claim 22, wherein said marker nucleic acid molecule has a nucleotide sequence selected from the group consisting of SEQ ID NOs: 3 through 463.

25. The method for diagnosing glaucoma of claim 22, further comprising a second marker nucleic acid molecule.

26. The method for diagnosing glaucoma of claim 22, wherein the cell or bodily fluid comprises ocular tissue.

27. The method for diagnosing glaucoma of claim 22, wherein the cell or bodily fluid comprises optic nerve cells.

28. The method for diagnosing glaucoma of claim 22, wherein the cell or bodily fluid comprises retinal cells.

29. The method for diagnosing glaucoma of claim 22, wherein the cell or bodily fluid comprises a bodily fluid selected from the group consisting of glaucomatous cell extract, fluid from the anterior chamber of the eye, blood, lymph, and serum.

30. The method for diagnosing glaucoma of claim 22, further comprising amplifying the complementary nucleic acid molecule obtained from a sample using a nucleic acid amplification method.

31. The method for diagnosing glaucoma of claim 22, wherein the nucleic acid amplification method is selected from the group consisting of polymerase chain amplification, ligase chain reaction, oligonucleotide ligation assay, thermal amplification, and transcription base amplification.

32. A method for prognosing glaucoma in a sample obtained from a cell or a bodily fluid by detecting a polymorphism in a promoter region of the optineurin gene, comprising the steps of:
(A) incubating under conditions permitting nucleic acid hybridization, a marker nucleic acid molecule, said marker nucleic acid molecule having a nucleic acid sequence that specifically hybridizes to a sequence selected from the group consisting of SEQ ID NO: 1 and complement thereof, and a complementary nucleic acid molecule obtained from a sample, wherein nucleic acid hybridization between said marker nucleic acid molecule and said complementary nucleic acid molecule permits the detection of said polymorphism;
(B) permitting hybridization between said marker nucleic acid molecule and said complementary nucleic acid molecule; and
(C) detecting the presence of said polymorphism, wherein the detection of said polymorphism is prognostic of glaucoma.

33. The method for prognosing glaucoma of claim 32, wherein said polymorphism is a single nucleotide polymorphism.

34. The method for prognosing glaucoma of claim 32, wherein said marker nucleic acid molecule has a nucleotide sequence selected from the group consisting of SEQ ID NOs: 3 through 463.

35. The method for prognosing glaucoma of claim 32, further comprising a second marker nucleic acid molecule.

36. A method for diagnosing or prognosing glaucoma in a sample obtained from a cell or a bodily fluid by detecting a polymorphism in a promoter region of the optineurin gene, comprising the steps of:
(A) incubating under conditions permitting nucleic acid hybridization, a marker nucleic acid molecule, said marker nucleic acid molecule having a nucleic acid sequence that specifically hybridizes to a optineurin promoter sequence or its complement, and a complementary nucleic acid molecule obtained from a sample, wherein nucleic acid hybridization between said marker nucleic acid molecule and said complementary nucleic acid molecule permits the detection of said polymorphism;
(B) permitting hybridization between said marker nucleic acid molecule and said complementary nucleic acid molecule; and
(C) detecting the presence of said polymorphism, wherein the detection of said polymorphism is diagnostic or prognostic of glaucoma.

37. The method for diagnosing or prognosing glaucoma of claim 36, wherein said optineurin promoter sequence comprises SEQ ID NO: 1 or a fragment thereof.

38. The method for diagnosing or prognosing glaucoma of claim 36, wherein said marker nucleic acid is capable of specifically detecting a single nucleotide polymorphism.

39. The method for diagnosing or prognosing glaucoma of claim 36, wherein said marker nucleic acid molecule has a nucleotide sequence selected from the group consisting of SEQ ID NOs: 3 through 463.

40. The method for diagnosing or prognosing glaucoma of claim 36, further comprising a second marker nucleic acid molecule.

41. A method for detecting the presence or absence of a SNP sequence variation in a sample containing DNA, comprising contacting a labeled nucleic acid capable of detecting a single nucleotide polymorphism selected from table 1 with the DNA of the sample under hybridization conditions and determining the presence of hybrid nucleic acid molecules comprising the labeled nucleic acid.

42. The method of claim 41, wherein the sample containing DNA is derived from a human with elevated intraocular pressure.

43. The method of claim 41, wherein the sample containing DNA is derived from a human without elevated intraocular pressure.

44. A method for detecting the presence or absence of an optineurin promoter sequence variation in a sample containing DNA, comprising providing amplification reaction primers that direct amplification of a selected nucleic acid region containing said sequence variation within said optineurin promoter, amplifyng the nucleic acid defined by the amplification reaction primers, and determining the presence or absence of said sequence variation.

45. The method of claim 44, wherein the determining the presence or absence of said sequence variation comprises sequencing the amplified nucleic acid.

46. The method of claim 44, wherein the determining the presence or absence of said sequence variation comprises a hybridization assay.

47. A method for determining the presence of increased susceptibility to a glaucoma, or to a progressive ocular hypertensive disorder resulting in loss of visual field in a patient, or the severity or progression of glaucoma in a patient, comprising providing amplification reaction primers that direct amplification of a selected nucleic acid region containing said sequence variation within said optineurin promoter, amplifying the nucleic acid defined by the amplification reaction primers, and determining the presence or absence of said sequence variation.

48. A method for detecting a polymorphism comprising: obtaining a sample containing human genomic DNA, providing a nucleic acid molecule capable of detecting a single nucleotide polymorphism located with an optineurin promoter, and detecting the presence or absence of said polymorphism.

49. The method detecting a polymorphism according to claim 48, wherein said polymorphism is selected from table 1.

50. A kit for determining the presence of increased susceptibility to a glaucoma, or to a progressive ocular hypertensive disorder resulting in loss of visual field, or the severity or progression of glaucoma in a patient, comprising a labeled nucleic acid capable of detecting a single nucleotide polymorphism selected from table 1 and a means for detecting hybridization with the labeled nucleic acid, and instructions for using said kit.

51. A kit for determining the presence of increased susceptibility to a glaucoma, or to a progressive ocular hypertensive disorder resulting in loss of visual field in a patient, or the severity or progression of glaucoma in a patient, comprising amplification reaction primers that direct amplification of a selected nucleic acid region containing a characteristic nucleotide of an optineurin promoter SNP sequence variant and an enzyme for amplifying the region containing said characteristic nucleotide.


SEQUENCE LISTING (463 sequences)

SEQ ID NO: 1 - DNA, 5054 bp, Homo sapiens
  allele 391: single nucleotide polymorphism (SNP)
gtacacctagaagtggaattgctgggtcatatcataactctctgtttaactttccaaggaactcatcctcggaatatttggaaccagtgatgaactgaatcaaactaaagctgagacaaagtccagaccaaggtcaaccatagggcagatgattcatgcagcgaccacaccagtggcctcacaggagcaggggcacaccctttgctgcagcagtccccaacatttttgacaccaggaactggtttcatggaagacaatttttccatggatggtggtgggtgggggggtggttttgggatgaaatgggtccacctcagatcatcaggcattagagtctcataagaagcacgcaacctagatcccttgcatgttctgttgacaatagggttcacgctcctatgaaaatctaatgcagctgctgatctgacaagaggcggagcttaggccataatgctcacccacccgttgctcacctcctgctgttcggtctagttcctgagaggccacaggccagtactggttcaccacccggggtttggggacccctgctttattggacataattattaggtcgtgttctttttggtggtgtttgtacagctctattgaggtataatccacatgccataaaattcaccccatttgtaaatgtatgattcatggctttcaattacacttaaaaagttgtaaaaccatcattacaattcaaatttagtatatttccatcatcccccaaaaatcccctcgagttcctttgcagttcaaagccacccccaatttcaggcaactactggtctgatttctgtctttttctactttccttttctggacatttaatgtatatggagtcatagcatatgtagtctttggcatctggggtagcaagtacgaatattagtctaccacctcagatgcacataaaaatattacatatcttttcttttcttttccttccttccttccttccctccttcctttctctctctacttccttccttccctccttcttacctttcttccttctctctctctctctctttctttttggacagagtctcactccatggcccaggctggagtgcagtggcaccatcttggctcagcgcaacctttgactcccaggctcaagcaattctcctgcctcagcctctcaagtagctgagattacaggcacgcaccactactgcctggctaatttttatatttttagtagagatagggtttcaccatgttagccaggctggtcttgaactcctgacctcaaacgatcctcccaaagtgctgggattacaggcgtgagccaccgccctgggcctctttactttctttaaacccagttctgcaggggtgtacggaaacctattttcgggcaccactggggtctggagaggggaggtctccttccctacggccatgcaaaactccaggagggcttttggtacccattgaagtaagggccatttatttttcagcccagcaacattgccactgataccctcattatcaaatggttcttctagggaacagtctctgctgtttccaatgacaagcctgggcagcagaatctgcgggaggttcccaaagtccagtaggtgcatcccaagagcttgctgtctgtctgggtgctgcagggactgaggcttgagtccttgatgctcataagaccaccatcccactcctcctcccaatctgggggcgggggagtcactcctccctccaactgttgtgaaagcctccaccccacccagctctggctcttcctccaggacatctggggtagatcatggatgtattgagatcaggctttctcaaaagacaagaagaaaggctgtacttctaagagctgttgccaggagtccagccaacgctcctgaaggtaggaagcccaaagggactcgttgctaactccaaacagaggagattggggtggaaagggaacacaaggaacatcaaacccagattaggatctcactaaaaaccttcccacactgctctacatttacccaccacaaaaccacatcaacaaatcagctaagagcatgctattatttcagttttttcgctgcatttagattccatctacccatggaagtgtgcaggaagatggagtcaccaaacgggatgatccaggctaagaaacagaaccggctctaacacaagcaacagcaacaaacaccatgagccaggcgttcttctaggtgttgaagacgtatttcctctttaatcttctcagcatccttaggtgagggctgtgggtccagaggccttatctaaaatttttgggtggctgggcaccgtggctcacacatgtaatcccagcactttggaaggccgaggcaggtggatcacccgaggtcaggagttcaataccaggctggtcaacatggcgaaacctcatcaatacgaaaaatgcaaaaattagcttggtgtggtggcacacgcctgtaatcccagccacttgggaggctgaggcaggagaatcactcaaacccaggaggtggagattgcagtgagctgagattatgccactgcactccagcctgggcaacagagtgagactccacctcaaaaaataaaataaaacctttggggcaagctctgctttagagtccagaattctggggattttcaaaaggctattcaataaatgggatttatatcacataacaccctgacactgtctgacgcagttctcctatcaactattcgattttccttcacaaaacaaatttaaaaatcacatcaagggatctaaataaagactgtaaatagctttccatcagttgggtctggtcagaaaagaggtttggtccttagaactttctggatttgggagtgtactatactccccattttacagataaagggaatgaggaagggtaagatgaagtaacttggtcaaggtcctacagctaagaagtggttgtcgggggagtgtgtgtgcatgtgtgtgtgcagtgcttcagggcaccccccaccccgaccccaccactgagagcaaggaatcaggagaaaacaactttgactgctttctgtaccagaaactcacctcgagcctcccacaccaaagccatgggcagcttgtgggtgaccttcttctcttggctctgagtttcactgatgctcattttaattcactttcatagtgttgttttgttctcgtttttgtttttgcttgagacaaagtctccctctcacccaagctggagtgcagtgctgcaatcacagctcattgcagcctctccctcctgggctcaagcgatcctcctgccttgacctcccaaagtgctgggattacaggtgtgagccaccgtgccccacctatagggttttaaacagtaaaaggagcctagtgaagtacgacttaccgcaggcaccccttacaggccccggggggacccttttctgccgatcccagggtacagctgtgacaccgtcttttctgcctggattatcccagtagataaacaaaaattagagatcgtcattccatttctctctgtatatatttttccaagcccttttcatgaatgatcagttatttcctgcactgattttttttttttttttttttttttgagacggagtctcactctgtcacccaggctggagtgcagtggcatgatctcggctcgctgcaagctctgcctcccgggttcaagcgattcttctgccttagcctcccgagtagctgggactacaggagagtaccatcatgcccggctaatttttgtatttttagtagagacaggctttcaccatattggccaggctggtctcgaactccggaccttgcgatctgcctgccttggcctcccaaagtgctgggattacaggcgtgagccaccgcgcctggccctcctgcactgatataaaaagaatttttttaaattctctatttctccccactcccacccccaggctcactccttataaagcagcctctagcctcatctgcccctcctacaccacaccaactcgagggcccggaattgggtctggggcagtcgctgacttgcttgcttctctgccctgctcttggggttagcctcaggggcagggttgagagtcaggcttggccaggcagcaggaggtccagacagcgaagcagaatccttcggagataccaggagagggcgcatctgcctttttcctgtttcagattaggttttgttgttgttgttttgttttttttctctccctctctccctccctccctctctccctccctctctccctctctccgtccctccctctctctctctctccccctccctccttccctccctcccatccctgcagcgctacccgggtactctggatgcacatagggcggctctcgctcctaccttgtcatcctgctgtctaatccgggggcagcttccctcctccacaccagcagaggctattcttcagcaacaagaatagccgagcctattcgtccgcaacaagagcccaagaagcatcctgcaggctttctgctttttgagtgtattttaaagcaaaaacgagtggaaagctatgtatgctcagttaactatgtctagatgttaaccttttttcaaaaaacacagatggaggcctccctccgaggatgcctggcattctcctctttctgtgggcggcagcgaccccctgcggctccagcctccactacgggatctgcgggaagacacggggaagacgaactccgcacactgcatttgattaatgatttattttgattaacgccgtcacagtgacgccttagagcagtccctgttcacccgggtcccagcctcgaccccgcacggacagcgagggtgggtagctgggggcggacgcaggaaagaggaggggcggggccttggtcgggtggggtatggaatgggcagggtgggggggatgggcggggtatgggatgggcggggcccgggaaattccccggcgcgggcagggagcggctggctgtcagctgagccgcgctgggcggggtcgccaggccgcgcatcagccctaggcaccccagtcccggctgccccctccgccaccgccgccgcccgccggcaggttcc

SEQ ID NO: 2 - DNA, 46951 bp, Homo sapiens
  allele 391: single nucleotide polymorphism (SNP)
gtacacctagaagtggaattgctgggtcatatcataactctctgtttaactttccaaggaactcatcctcggaatatttggaaccagtgatgaactgaatcaaactaaagctgagacaaagtccagaccaaggtcaaccatagggcagatgattcatgcagcgaccacaccagtggcctcacaggagcaggggcacaccctttgctgcagcagtccccaacatttttgacaccaggaactggtttcatggaagacaatttttccatggatggtggtgggtgggggggtggttttgggatgaaatgggtccacctcagatcatcaggcattagagtctcataagaagcacgcaacctagatcccttgcatgttctgttgacaatagggttcacgctcctatgaaaatctaatgcagctgctgatctgacaagaggcggagcttaggccataatgctcacccacccgttgctcacctcctgctgttcggtctagttcctgagaggccacaggccagtactggttcaccacccggggtttggggacccctgctttattggacataattattaggtcgtgttctttttggtggtgtttgtacagctctattgaggtataatccacatgccataaaattcaccccatttgtaaatgtatgattcatggctttcaattacacttaaaaagttgtaaaaccatcattacaattcaaatttagtatatttccatcatcccccaaaaatcccctcgagttcctttgcagttcaaagccacccccaatttcaggcaactactggtctgatttctgtctttttctactttccttttctggacatttaatgtatatggagtcatagcatatgtagtctttggcatctggggtagcaagtacgaatattagtctaccacctcagatgcacataaaaatattacatatcttttcttttcttttccttccttccttccttccctccttcctttctctctctacttccttccttccctccttcttacctttcttccttctctctctctctctctttctttttggacagagtctcactccatggcccaggctggagtgcagtggcaccatcttggctcagcgcaacctttgactcccaggctcaagcaattctcctgcctcagcctctcaagtagctgagattacaggcacgcaccactactgcctggctaatttttatatttttagtagagatagggtttcaccatgttagccaggctggtcttgaactcctgacctcaaacgatcctcccaaagtgctgggattacaggcgtgagccaccgccctgggcctctttactttctttaaacccagttctgcaggggtgtacggaaacctattttcgggcaccactggggtctggagaggggaggtctccttccctacggccatgcaaaactccaggagggcttttggtacccattgaagtaagggccatttatttttcagcccagcaacattgccactgataccctcattatcaaatggttcttctagggaacagtctctgctgtttccaatgacaagcctgggcagcagaatctgcgggaggttcccaaagtccagtaggtgcatcccaagagcttgctgtctgtctgggtgctgcagggactgaggcttgagtccttgatgctcataagaccaccatcccactcctcctcccaatctgggggcgggggagtcactcctccctccaactgttgtgaaagcctccaccccacccagctctggctcttcctccaggacatctggggtagatcatggatgtattgagatcaggctttctcaaaagacaagaagaaaggctgtacttctaagagctgttgccaggagtccagccaacgctcctgaaggtaggaagcccaaagggactcgttgctaactccaaacagaggagattggggtggaaagggaacacaaggaacatcaaacccagattaggatctcactaaaaaccttcccacactgctctacatttacccaccacaaaaccacatcaacaaatcagctaagagcatgctattatttcagttttttcgctgcatttagattccatctacccatggaagtgtgcaggaagatggagtcaccaaacgggatgatccaggctaagaaacagaaccggctctaacacaagcaacagcaacaaacaccatgagccaggcgttcttctaggtgttgaagacgtatttcctctttaatcttctcagcatccttaggtgagggctgtgggtccagaggccttatctaaaatttttgggtggctgggcaccgtggctcacacatgtaatcccagcactttggaaggccgaggcaggtggatcacccgaggtcaggagttcaataccaggctggtcaacatggcgaaacctcatcaatacgaaaaatgcaaaaattagcttggtgtggtggcacacgcctgtaatcccagccacttgggaggctgaggcaggagaatcactcaaacccaggaggtggagattgcagtgagctgagattatgccactgcactccagcctgggcaacagagtgagactccacctcaaaaaataaaataaaacctttggggcaagctctgctttagagtccagaattctggggattttcaaaaggctattcaataaatgggatttatatcacataacaccctgacactgtctgacgcagttctcctatcaactattcgattttccttcacaaaacaaatttaaaaatcacatcaagggatctaaataaagactgtaaatagctttccatcagttgggtctggtcagaaaagaggtttggtccttagaactttctggatttgggagtgtactatactccccattttacagataaagggaatgaggaagggtaagatgaagtaacttggtcaaggtcctacagctaagaagtggttgtcgggggagtgtgtgtgcatgtgtgtgtgcagtgcttcagggcaccccccaccccgaccccaccactgagagcaaggaatcaggagaaaacaactttgactgctttctgtaccagaaactcacctcgagcctcccacaccaaagccatgggcagcttgtgggtgaccttcttctcttggctctgagtttcactgatgctcattttaattcactttcatagtgttgttttgttctcgtttttgtttttgcttgagacaaagtctccctctcacccaagctggagtgcagtgctgcaatcacagctcattgcagcctctccctcctgggctcaagcgatcctcctgccttgacctcccaaagtgctgggattacaggtgtgagccaccgtgccccacctatagggttttaaacagtaaaaggagcctagtgaagtacgacttaccgcaggcaccccttacaggccccggggggacccttttctgccgatcccagggtacagctgtgacaccgtcttttctgcctggattatcccagtagataaacaaaaattagagatcgtcattccatttctctctgtatatatttttccaagcccttttcatgaatgatcagttatttcctgcactgattttttttttttttttttttttttgagacggagtctcactctgtcacccaggctggagtgcagtggcatgatctcggctcgctgcaagctctgcctcccgggttcaagcgattcttctgccttagcctcccgagtagctgggactacaggagagtaccatcatgcccggctaatttttgtatttttagtagagacaggctttcaccatattggccaggctggtctcgaactccggaccttgcgatctgcctgccttggcctcccaaagtgctgggattacaggcgtgagccaccgcgcctggccctcctgcactgatataaaaagaatttttttaaattctctatttctccccactcccacccccaggctcactccttataaagcagcctctagcctcatctgcccctcctacaccacaccaactcgagggcccggaattgggtctggggcagtcgctgacttgcttgcttctctgccctgctcttggggttagcctcaggggcagggttgagagtcaggcttggccaggcagcaggaggtccagacagcgaagcagaatccttcggagataccaggagagggcgcatctgcctttttcctgtttcagattaggttttgttgttgttgttttgttttttttctctccctctctccctccctccctctctccctccctctctccctctctccgtccctccctctctctctctctccccctccctccttccctccctcccatccctgcagcgctacccgggtactctggatgcacatagggcggctctcgctcctaccttgtcatcctgctgtctaatccgggggcagcttccctcctccacaccagcagaggctattcttcagcaacaagaatagccgagcctattcgtccgcaacaagagcccaagaagcatcctgcaggctttctgctttttgagtgtattttaaagcaaaaacgagtggaaagctatgtatgctcagttaactatgtctagatgttaaccttttttcaaaaaacacagatggaggcctccctccgaggatgcctggcattctcctctttctgtgggcggcagcgaccccctgcggctccagcctccactacgggatctgcgggaagacacggggaagacgaactccgcacactgcatttgattaatgatttattttgattaacgccgtcacagtgacgccttagagcagtccctgttcacccgggtcccagcctcgaccccgcacggacagcgagggtgggtagctgggggcggacgcaggaaagaggaggggcggggccttggtcgggtggggtatggaatgggcagggtgggggggatgggcggggtatgggatgggcggggcccgggaaattccccggcgcgggcagggagcggctggctgtcagctgagccgcgctgggcggggtcgccaggccgcgcatcagccctaggcaccccagtcccggctgccccctccgccaccgccgccgcccgccggcaggttccctggtcagcgtcccatcccggtcgggagttctctccaggcggcacgatgccgaggaaacagtgaccctgagcgaagccaagccgggcggcaggtgagccagggcagggggctgcagcggtgggcgagggggcggcggctccttcccggcgggcgcttccgcggcctgaaaacggtacccgccgccctgccccgcggcccggagcctgtcggggtgagggtggcgaggggtggggcggccctcccgcgagacggctgccctgggaggatccccaagcctcggcgggtcctgacctggtgggagagggtgggtagtgaacacagggtgggcccaagaagggtccagggccggcctcttggccgcaggtaacccctctacaagtcacccagagacttggaggtgacgtcctcccgcaggctgggaaggattccgcagccagttcctcgccataaggaggtggttacagacacgcctgtgggagacatgggaccactacccgagctgaatgaggtgcccagggtcggtgggaatccacatagactgctgtgtctaggagagtgtgtacactctatatagagagaatatttgatgaggccgagaaccccaaaggaattgtttttgccatcgaaatgcataagctacaggaagtaaacatcgctcaaagaaaatgaaacccaggttccgacctggcctccaccagcaggaaaactgttgtatggaaatgtacaccaaggaactgtttgtaaggatgaaatggaaaatgaaagcatagaaaagtaaggcaatgtgaggacctcagaagcctttggatctggttagcaaggtgcaggctgtgtgaacagaagaacccaccttgctcttttgacccagggagagaagagttagtggtgccaggctgcctcccaggacaggcccagttttggggcgggggctggagaagtcccagggcagaccccaaggccagggattgccctggagtggacaaggcaagtttaaatgtgacctgtagacagaggacgatcattgatttaagtcctggcataattcatctgtttttccactgggccaattttaggcgttctttatatattacgtacaacacaataaaagtaacagcttctttttttataaactaacctttaggttgcatattatgctaggaatataactaacctaacctccacccccccacccccccacccacccccggaaaaccaaactttattctcgagggcaactggacactgtattatgtcctgccgaacccccgcccttacttaggtagaggcgcataagtattctggccatgaaatacaccttctgttttatagagatttggcatatttgatatatgttctttggaatttatgaaatagaaaacccaaaggaaaaagaaaaaaattaaacttttttttaaaaaaatatttcatttagaaagaaaaaattaaaagtttttgttatttccactttgacatgtgtatgaggagcattagaatctgtgttatgaagtgatacccagatggctttgcagtgacagaaaacacttctaatttttcaaataagttagaaaggaggattttgagaatctggaagtgaatgccgtggaactgtggagaaactcctaacaatactggcatgctgagtttgtcacctacacagcagaaagcattttacaggtattagacttaaaaatacctcattatgctatattttcatttcatttcaaccaacatttactgcaagcctgctatgtgccagtctctagtaagaataacatggagagaaaatagacgggcagacgatagcagtaatttcagaatgggcggaggaggtggaaatgcagagtgttcttgggtgctcggagtagggaacagttcatatgacacaggacatcatatttaagctggacattgatctgtccagtggaagtttgccaggacacaaggggagccagacatttcgggcagagggcagaagggaggcaaggacgcagagacagaagaatgcagggaaccgtgagctctttaacgtgccttgggtacgagtgttttggtggcaggaggtgactctggtgggtcagttgggcctgggccctgaaaggccacgtttgtcttgttaagaaactgaaagtctgtcgtgtatgggaagggtggactcaggggaatgacctcatattgcaaaagaaaccatgagggcaagaggactgaaattgacagggctgaaagccagtcatttaggaggctgttaaaatgtattcaacagatatttaccgagtcctgccttctatgtggcaagtaatgttctcagtcctggggatacagcagtggacaaaataggcagaacccctgtcctttgtggaggtcaacatggctgtggtacagacaagataagggcctgaactaagcctgtggggatggagaggaggcagtggattttagaaacacttagaagggccaggcatggtggctcatgcctgtaatcccagcacttagggaggccgaggtgggtggatcacttgagatcaggagttcaagaccagcctggccaacgtggcaaaaccctgtctctactaaaaatacaaaaattagctgggaatggtggcatgcacctgtaatcccagctacttgggaggctgaggtgggagaattgcttgaacctgggaggcggaggttgcagtgagctgagattttgccacggcactctagcctgggcgacagagtgagactgagtcttaatttaaaaaaaaaaagaaaaaataaatatttaggaggtagaggtgatggcatttggtgacccattagacttggggaaaagggggctgggaagaatcaagccttactcccaggttcctgccttggggagctaggttaaagctgctgccattttctgtatattactaaaatacgaagtcacaatatcccttctagtgtgtaatgacgaggctgcttttaccccattagtaggatgcatactctaagttctttttccaaggtgggaagggtttagtcaatgcaatgttagaatcttaccccccttgtgaacccaaactcttcatagcatttgccagtagccaactcttgtctctggtcaaaaagcaagttttcactgtttaaaactcatttatcccatgtagggattgaacctacgaatacttcattagcaaatttgaagacttggccataccagggcaagggcctaaccttatactatgtgcagtgtccactggggggaagtcagtgtaaggcaggtaccatttgacacattatgactttctgaccactagagccatcccacagtggaacagacagtgcctgctaaggtcactgcactcgtgctctgtcattgaaatactcaagcctgctctataggatcatgtgccatcgaaaccagacaagacattcttgcattgaatatattggtctggatcatttctaaggccaactcagcaatcagatgtatgctcgtgtttacaaggtcaggtcattactagaattacttgatctctggaatttaaatgtatatacacacacatgcacacacacacacacacacacacacaatcaggaattgagatagcagatctgggaataaaatctaggggacttgcttttggccatccatctactaggcagtcctcagtcttcctcattggtggaatcttgacaaatcctgtgctcaattaattcatccttccatcattaaatatcacttttgcctagaaataacatacttcacatctaggtcagttgattatgggaaactctgttatttgatagaatcttagaataaatggcttagttccaatttctgtaaccttcgaagacatgtgcaaggatatgtaatgtatctctgtctttcctcagttgaatgtagctcaaggtgatgttaaaaacagagaatgaagctttctgtaactggcattccaccgacagatatttcatatgtgcactgcacagtgatagctcatagtgatgtcctaagataacctaagaattatgaagtgccctttgagtcatccaaaatcagttaaattagtttttttttctttttttttttttttttttttgtgagacagggtctcactctgtcacccaggctggagtgcagtagcatgatcttagctcactgcaacctctgcctcccaggttcaagcgattctcatgcctcagctcccgagtagctgggactacaggcatgcaccactacacccagctacttttttttttttttttttttactattacaaaatttatttaacaaaaagtctaatatgaaaatgtacatgacctaacttttacatcatagtaaaacaggccctatggagagaggacatgggtttctctgctgaacagccattttttatactcattccaaggcttctaacatgaggatactgtttcctcgtattaccaccattccagtattgttctgttgcccactagtcgccatctccacacattcatctatcacaagattcataaagggatcaagtccctgcaatattccttggacgtgtctgccaccatttaatttcaatgataacttcttgtccataaatcccttctagccatctggttagaacatgatgctattcgagatgttcaacaaaaggtggcattcgagatcttcaaggaacgaagaagggacagcatgggggtgaccaggggcccgaccgcaggaccgggaggcgagtcggccagaaagaggtgcagtggctgctggtggtaactacgccgacgtgcgagctctgctactaatttatgtatttttagtagagatgggatttcaccatgttggctgggctgatctcaaactcctggcctcaaatgatctgcccaccttggcttcccaaagtgctgggattacaggcataagccactgcacccggcatcaattaaattacttttagtaaaattttggtattaagtatttacatgcttatatttaccgtttatgctaacttcttatgtttgatgacatttgaattacaaaatattttttctaacaatacacaatatcacagaatttgctgtcattaggacagttatagctattcatgagagtgactggcctgcccgtgatacttggtgtatatgcattgaagggtggggtttagattgtggtctttctagaaactaaagaatatttttcacagagttcataaggattatcatttgctggatgagtcaataaataccactagaggtcatgtgacttcccaaggccacacatctggctaatggtacaaatgtttttttctgtggcctcccttagcacgaccccacttttttcctcgtatgcttataaaatccgggcaatgtgagaagtcagaaacactgatgtaacaactagaaccagttttaactttattatggtggtatctgtaggcttggtggaggcataaaaggtcatggaggggcccggcaccgtggctcaagcctgtaattccagcactttgggaggctgagatgggtggatcacctgaggtcacgagttcaagaccagactggccaacttggcaaaaccctgtctctactaaaaatacaaaaattagccgggtgtggtagcacatgcctgtgatcccagctacttgggaggttgaggcaggagaatcacttgaatctgaaaggcagaggttgcagtgaactgagattgtgccactgtactccagcctgggcgacaaagtgagactctgtctgaaaaaaaaaaaaaagttcattgaggaaaaaaatcttaacagttttcaaagaaacgtctctctcttttcttccctctcttctcccctccccttcctttcattttcagaaaaaaaggagaaatttgtgcatataataagatgaaaccaaacacacttctaaaaatctgcttagatcctttctttaaaaaaatctgttgaatattttcatggcctttgcaaaatatagtataatcactgaatacctaagaaaaattaactgttggcaagtaaatgatacagatgacaaggattttctcttttaggtctattacctagaattttagtattcctctgcaaattaggtaggaacactgttactcagtaaaaccctattttaatgagatgattctgggtacaaaaaaatgaattttacagaaaattagaaactgatggtctattcagattatttttgtgagaagaagagcagtctgttctagtcacctaatgctaagctatggaacacttctgcattatacctgttttctaagtgaatttgggtgtgtgacacatagatacaaaaagttcaaaaatgaatcctatggtttatcagtgttttctgcttcgtaagattgccatcaccctcattacatgactacatgagaatgcccctcttggtactagctcttgtaggaagaagtggcacaggctgtactgtcacagggtgtggtaggtagctctgtctctgattacctacaagttcctcacaccaaaaggaggttaccaggtattgcttccagaagatctacagaaagcccaaggtattgcctcctgtggccaaggacgcaacaccacagtggtgcattttgttttattttaattgatggctattaaatgcacacatagatatcatgacacagttacatgtcagaaacaggcagtgctcttatctctatcttccagggactgatatcatgtgtaatggattcttcacaactcagggttcacacaggctccatagtcatgagtggctgatggatctcattgagaccagtgcctttcccccgttagaaatgggatgctcgcttccaagttctctcctcaccccttttgtgcatgcgtggctttctgcctgatcacagttgtatagtgtacattggtgcttaatacctatttattggatagatggattttaaattattttctaaggtccaggagcagtggcttatgcctgtaatcccagcacttttggaggccgaggccagcagatcacctgaagtcaggagttcgagaccggcctggctaacatgatgaaaccccgtctctactaaaaatacaaaaattggccaggcatggtggctgacgcctatagtcccagctacttgggaggctgagtcaggagaatcacttgaacccaggaggtggaggttgcagtgagccgagatcgctccactgcactccagcctgggcaacagagagagactctcaaaaaaccaaaacagtctttttttttttttttttttgagactctgtcgcccatgctggagtgcagtggcacgatctcggctcactgcaagctctgcctcctgggttcacgccattctcctgccgcagcctccggagtagctgggactacaggcacctgccaccatgcttggctaattttttgtattttttttattagagacagggtttcaccgtgttagccaggatggtctcgatctcctgacctcgtaatctgcccgcctcagcctcccaaagtgctgggattagaggcgtgagccaccgcgcccggctcaaaaacaatcttctaacagtaaacatcctggaagtaaataatatgctttttcaggcatgtctctggactttggccattcttgacctttgtagaatgcgtgtgtgggccatgtgtacacagcgtcagcggggaggagttgcctttgctatgtattgctttataatctatccccatcagctcttgaagaaaatacatcattttcaccaagtgtgaaggtggaaatttgtgctgaaacatgtttgcctgggtggttggtaacacagaagcagagtggggatttactcattggtcttgcatttctatgtccacatggatgcctctacaaaacaaaatgatatgtgtaaaaaaatttcagaccatgcaatacctaaagatatcttagtcttttcagtatgcattgaaaaatagactattaaagcctaagtactcagtaatataactttgttgttttacaaggtgtggctttgatagctggtggtgccacttcctggccttggatgagccgtacgcctctgtaaacccaacttcctcacctttgaaacagctgcctggttcagcattaatgaagattagtcagtgacaggcctggtgtgctgagtccgcacatagtaagcattcaaagaatgttaattctccctttcttcttaaccaaaaacacaataaactaaatataggttttaaaacagcttttttgagatagaattcacttaccatgcaatttacttgaaataaggagatttgaatttgaagatttgcatgggaaatttttttcatgataccttgcccatgtctacatatatcctgatcatcatgatgtcataccctcgtctccctcccccatttcccaaatccttattgtaccctttttttttttttttggtttgagacggagtctcgctctgttgcccaggctggagtgcagtggcgcgatcttggctcactgcaagctctgcctcccgggttcacgccattctcctgcctcagcctcccgagtagctgggactacaagcgcccaacaccaagcccggctaattttttgtatttttagtagagacggggtttcactgtgttagccaggatggtctcaatctcctgacctcatgatctgtccgcctcggcctcccaaagtgctgggattacaggcgtgagccaccacgcccggccctcattgtacccttttatacacccatacacacacacgcacacacacacatgcacacatgcgcgtgcacacacacacacacttttctgaagctacatataccttttttgtttaaaaggaagaatcaaaaatgtccaaaatgtaactggagagaaagtgggcaacttttggagtaagtattagcaatcgccaatgggtttgtgggactcccggggaccccttgtggggcgggggacagctctattttcaacaggtgacttttccacaggaacttctgcaatgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaaggtgagcagggctggcccctgtgtgccccattcatcctgggcctgcaagaaatgccatccctttgcactaaggcttggtggtgagctcccttctccccgtttccataggtggtagctggtggggaagcacaggatttagcatttggcaaggctaaatctgttctgatttttacttttggaaacaggtacaagtaaaaactgtgtgtatctcaaggaagtagcataatgatatttagcccattcaaaaggaaaaagaggctgggcgtggtggctcatgcctgtcattccatcactttgggaggccgaggcagaaggattgcttgagtacaggagttcaagaccagcctgggcaagatggcaagacctgatctctacaaaaaaattaaaaaaaaaaaaaaaaagctgggcgtggtggtgcacgcctctggtcctagctactggggatgctgaggttggaggattgcttgagcctgggaagttggagctgcagtgagccatgatcgtgccactgcactttagcctggatgacagagagagaccctgactcaaaaaaaaaaaaaaaaaaaggaaaaaggaagaaaggctgctatggttccagagttagtcctatatattaccttattaagagaaagcatcctggtatctcaagatggctttgggcaggaccagtatttgaatctaggagtagtaagaacttccttagctcctagtaaccatagatatttagatatttgtgctgtagtggcggtacccaaatccactttattttcttgggatttttaaggactagaaatgatgttcatcccgctagtcttttctgtaagcaaaaaccacttcgtctttttgctgctgacccttgggccaaggctaagcatggcatctttcaattcagagccatgtggtcaagtggactagagggagatttggttcatcagatcaagtccactttcctggtgtgtgactccatcactctgaacctcctgcagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggtgagcagaccgatccattgtgatgttgttttttttttttcccttgacatttgcagtggaatcttacgtgtctagactcctagatcaaaacctttcatggttcagtctggattggtgttttgcctggtcttggaagaagtgcttttgctgaaaagattggttgccctattaagggtcatggataatctcttttagaagaaagaaatttgtaaagctttgaccgtactgattgtaggcaaaagaacagtaaggttataaatcattgtattgtattcattatagatggtgcagatgggcctctgcctagaaccaacaattgtttttagtttgtctttgatataaaaaatatgtttaaaaaacccattactcagaatttttacttgttgaccttgtctgttctctcagtctaaaatggagattattcactttacattttcctttttaaaaatgctttggaaaatgtcatgttgtggtaggaggctatcgcattgccacagatgaaaagagaaagagacacatttttcttaacccaaagaacctggaaaaatgtgctcatacctgggagtggatgtcaaagatgatagtaatgacacaacccagaacaagtttgaaatccccactgggcgtggtttgttaaggagtcctctgctgtccatgccaagtgtggggagaatgggtgtgggtgggctttgatgggaggaaaagggcaggaggttgggtggtgggaacgttttttccttcctttctggaattttagacacagcttatgagtccactgttgccaaagtgtggttgattatttctatgatatcagattattccagcatggcaaggaaggctttctttcttgtcatgaagttactttaaattttgattatttgaatacaataaaataatgtaaaaatttccattttaaatgttatgtcaaaataaatcgtgggctaggcacggtggctcacacctgtaatcccagcgctttgggaggctagggtgggcagatcacctgaggtgaggagttcgagaccagcctggccaacatggcgaaaccccatctctactaaaaatacaaaaattagccaggcatggtagtgcgtgcctgtagtcccagctacttgggaggctaaggcaggagaattgcttaaacccgagaggtggaggatgcagtaagctgagatcgcgccattgcactccaatgggcgacagagtgagactctgtctgggaaaaaaaaattgtcaggtaaaagctgaaattttctacattaaagcacaaggcataaaggtgttgagaaacttccttgtcagtaggtgtgggggcaattgagtctcatggccaggtccttagtttgatttgtatttgttttttgactgtttttttttttttttttttttgagatggagtcttgctctgttgcccaggctggagtacagtggcataatctcggctcactgcaagctccgcctcccaggttcacgccattgtcctgcctcagcctcccgagtagctgggactacaggcgcccgccaccacgcccagctaatctttttgtatttttagtagagacggggtttcactgtgttagccaggatggtctccatctcctgaccttcatgatccgcccacctcggcctcccaaagtgctgggattacaggcgtgagccaccacgcctggcttggcttttttttttttttttttgagacagggtcttggcagtcttaaactcctgggctcaggcagtcttcctgcctcagcctcccaactaatggggactacaggtgtgtgccactacacctggctaattattaaattttttgtaaagatgggggtcttgctatgttgcccaggctggtctcaaaatcctggcctcaagggatcctcccacttcagcctcccagagctctgcgattaagggcatgagcccatggtgcccagccttagtttgatctgttcattcactttactccttgtcatctccaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgtgagtttttggttttatttttgttttgagcaaactataaagcctcccctggaaagatgaaacaaataccactttttcttgtcaacacaagccaaggattgaggaaattccagtgtagcaaagataaattggctctcattttctaagtatagcataatgcatgtaagggttatcatagctaaaatggaaaaatattaattaccttttatgatgaaagctgtagtctttttttttttcttcatcatgtcctggcaaattgaacatttttgtgaccagaaaaggaaaaaacccacacgaacatgaactttctgtcatttttcaaactaggtctcaaagctgtattccgcagttcacttaagggagcgcaaacatattttcacaacagaaccctctttttttgttttgagacagagtcttactctgtcttcccggctggaatgcagtgatgtgatctcggctcactgcaccctctgcctccggggttcaagagattctcgtgcctcaacctcccaagtcgctgggactacaagcgcatgccatcacacccggctaactttttgtatttttaataaaaaagacaggtttttgccatgttggccaggctagtctcaaactcctggcttcaagtgatccacccgcctcggcctcccaaagtgctgggatgacaggcgtgagccaccgcgcctggccaacagaaccttcttttcaaacaaagtggtatgaggaaccctgatacattaaaaagaagaagaggagaaaagaaagagcagaactgctctggttgtaggttgagggagtgtcctggcttttccttccctttcaaaagcagctactcaggagcctctgagaactgagtttgaagccattgctatcaaaatcaaatttctctgcaaccccagatgaagtgggctaagcgagggggccctaagctcttgagaagcatctgttacaactgtgcctgggcataggggcagccctattgaagagcagagcaggtcgatgcaccagctgggggcctgtccttcattgctactaacaaagattagacagggagaaagatagacaaggataaaatcctctgtagtatagatggtcactttcgatgagtcagagtacatatctgatacaggaaaagggactggccgggtgtaatggctcacgcctataatcccagcactttgggaggctgaggaggcaggatcccttgagcccaggagttcaagaccagcctgggcaacctggcgaaaccctgtgtgtacataaaaaaaaaatacaaaaagttagctaggcacattggcacacacctgtactcccagctactcaggaggctgaggcaggaggatcacttgaacctgggagtttgagatgctgcagtgtgccatgattgtgccgctgcactccagcctgggtgacagagcaagactctgtctttaaatttaaaaaaaaaagaaaagaaaaaaaattgtgtccttggaagagaaaaatgtacattgtagataagtcaggaggagtggggaacaacttgcaaaaaagctcacctactggttatatctgaaatatgaatgtctgactgtctttgctttctgatttatttgctgcaatagagttaggaaacagctctgaataaccctgccatccattccccctcatacatttcagtggccaaaatcaagataattaaaatgtcaattgaaaagcgtattttgccagagtaccccttctgcaagtgatcgaagattactgagacagaagtttaaccaaagaaacttacccttctgtcaataaccgataagctgcaggaaacccaacagcatatgagaagtttccagatgaagcttcttccttcagggtgcatggttagcatctcattctccctgtcagatgagtgacagttatttttagtgtaaacacgttttgcatgcgtttcttctgtgaactggaaagcactgcccaagatttaggaagaaacatcaaaaatatctagaacccccatggccccaagcagagaaaaaatatatgtatctatgttttttataatagatatatctgtatttatattatataatgaatatataatatattctataatatataatgtagaatatattctacaattatataattgcatatataattatataattatataattatataattatatatacatatatataattatataattatataattatataattatataattgtataatatattctacaattatataattgtattatatacacttacatatgtatctgtattttttataatagatatatctatatttatatatatttatatatagatatatctatatttatatatatttatatatagatatatctatatttatatatatttatatatatagatatatctatatttatatatttttatatatagatatatctatatgtatatatatttatatatatagatatatctattttatatatttttatatatattatatatatatataaatttataatatagatatatctatatatttatatatagatatatctatattttatatatatatttatagatatatctatatttatatatatatttatatatagatatatctatatatatctatatttatatctatatttatataatgaatacatataatatattctataatatataatatataatataacatagaatatagaatatattatacaattatataattgtataatatatatacacttatatatgtatctgttttataatagatatatctgtatttatattatagaatatatataatatagaatatatataatatattctatattatataatatagaatatatataatatattctatattatataatatagaatatatataatatattctataatatataatatagaatatattatacattatatattatacaattaatatataattacatatataacattatatatttatataatatatatttctatatctaaatatctaaatatattatatatatatttatacgtatatacctagaactatctttgtgcatttttgaagattttccctgggagcttattggaatataaatgtctttcaaatcctgtgggtcttagactatcttgttccctaagtgacctgtggtgcatacaaatttctaatgggaaccaacttggccaagatggtgctttgtgaatctcattcacagaaactgcctcttttttaactttacctcagtgagttctagcattttgcattttaaaggaaggatatgtggagttgtcaccagctctgtatgaccttaaccttgagaaagagggaactgccaaggaaagggaggagcagataagctttcatgtttacagagtcaggtagaatgtgtatggcgagatgaaactgaccttcacgccttagctgggatatttataatcccgacagggcgtgccaggtgaggggagggtacgtttccatttcctctgagccaccccgtttaaacagtgcacatctgaatgtttggaagcttccttgggttgcatgtcacaaaaattcatcttttgtctttttcttcttttgacaaagaatttgtcttgtagacatattgtgttaaatcccttgcatttctgttttcacaggaaggagaagcagaagggtcagtaaaagaaatcaagcatagtcctgggcccacgagaacagtctccactggcacgtatgtgaaggaagactcgggctgtcaggcagacaggctgggcaggctcgtcactgggtgcttgtcaccggaggtcaaatgttgtgacctgaggaagtaacttctttatgatttataccaggatctttccagaatatttggtttgaatgctatttaatgttgcagctcaaactggcaaagattaaaaactgtttggttcctgtttggctcacactgactgctctgttctagtggtgtctcacctccagcagatgaaaagtgaaagcaaactggttctcaatcaagtcaatgatttgttcctaatcaaagacatgtttgctcattggttccccggtgccatttgacccagaccagcctgcccagcttccataagtgaaatattttcattttcttttccctgctacttcccagttataagctggcatggccaatactggaacatcttttgtaacaatgactgatagcactctcagtcattgtgggtgttgcctgaaagtgcccagatttcttatctgtggagtctcaagtgtacctgtcctatgtagatgtgaggaaacagacatcttaaatagtggcagggccttggggagggaggcagacctagaactgagcgcctgaacctcttgactctcgcaaagcagtgctcaccaaggagactgctagctcgccttctgcaagctgcttactcctgtaatcatctattcaagagacgattgtctgaaaagaactctaaggatctcttttttttttttttttttgagacggaatcttgttctgttgcccaggctggagtgcagtggcgtgatctcggctcactgcaagctccgcctcccgggttcacgccattctcctgccttagcctcccgagtagctggaaccacaggcgcccaccaccatgcccggctaattttttgtatttttagtagagatggggtttcaccgtgttagccaggatggtctcgatctcctgacctcgtgatctgcccgccttggcctcccaaagcgctgggattacaggcatgagccaccgcgcccggccaggatctcttatctcacagacggataagagatccatcttttttttttttttgagatggagttttttttttttttttttttttttgagatggagtttcactctgccactgaggctggagtgaagtggcgcaatctcagctcactgcaacgtctgcctcccgggtacaagcagttctcctgcctcagccttccgagtagctgggaatacaggtggccgccaccacgcccagctaactttttgtgtttttaatagagacagggtttcactgtgttgaccaggctggtctcgaactcctgacctcaggtgatcctctggtctcagcctcccaaagcgctgggattacaggcgtgagccactgcgcccagctatctcatagatttgtaaaaccttctttgtataacttgatgaatgtgcatatagaatgacttacaaaacgtgaaaaaaattgtcttccgttatgtttctaagcccttgtataaggaagaaagtaagtattagataatattctttcgtcaaacacagtaattggcataaacgaaagtaattcccttttttggttaacaattcttcacgtttcccccaaattgcttttgtcatatttaaagacgttcctggaagtagtgggaaataaaaaagccctggttgaaatatcaaagccacttcctcatcattcatatccagatgcaaagaccgcttcctcttttaagccacatggctatttttaaaataacagctctgtaccatttgtgagtatcgtaattagtttgagattgatttccaggtttggagttgaagcttcagagtcctggaaaccgggatttaatcctggctgtttattagctatgcgaacctgagtcggtgactgaaagagtagctgccgttagcatcatcatagtcatccctcatgttttgtaacagtgatcatgattctgtttgtcactgatatgtctcttgatttagtcagattttgaaatcgagaaaaatacttgacatatcaacagagcctccatttctctgctctcattatttgaaaccacaagtgaaaaaggttttctccccttgacttaagctgtgatggtctctgttaacttggagaaaggccagtggtctgtacaatgtgcctttatcttttgtctgactgcagtcccctttgagactagatctctggaaagcttggcaccttcagccacggctgcctctgctgaactgttccgtgagttttgtggtgtggtgtgaggtacacagtgactgtttggaggacgtgggtgtgtgcattgtaagctggcctctccagagcctcactgagtctccacaccttccctaggaagcatggaggagcttggcactgggggtcccaggaccagctgtgcttgttcactagttgagaattagttggagaatgttctggaaagcagttcctttaagctggtcccagttatattgggttactctcttcttagtctttggaatttttctgatgaaaaccttttaacctttatactgaacagggcattgtctaaatataggagcagatctgcagatggggccaagaattacttcgaacatgaggagttaactgtgagccagctcctgctgtgcctaagggaagggaatcagaaggtggagagacttgaagttgcactcaaggaggccaaagaaaggtatgaaataggttaacttgaaatatgtgtttttttaaaacagctttcctgagatataattaagataccatacagttcacccatttaaagtatacatttcagtgttttttagaatattccaggattgtgcaaccactgttactacaatataattttagaacattttttcccccaaacagcactcactgtctgctcctccaagcaatgtgctttctgtctctatagatttggccattctagacatttcatataaatggaattatacagtctgtggttttttgtgactggcttctttcacgtagcataatgtttttgaggttcatctacaacgtagcatgtatcagtacttccttttccttgctgaataaccttccattgtctatatatacaacattttgtttattcattcatcagttgataaacattagagttgttgccactttttacctattaggaataatgctgctatgaacagtgtgtacaagtttttactgggatatgtgtttttaattctctggggtatatcgttatgggtggaattgctgggtcatacggtatcgctatttcatattctaaggaaccagcaaatcattttccaaagcagctgcgccattttgcattcccaccagcagtgcatgagcattccactttctccacgtccataccaacacttgtttcttactgttttcactttgacttcagccatcctagtggatgtgaattggtatctcatagttctgatttgtatttccctagtgacagcgatgttgagcatcttttcatgttgaccgtttgtttgatttggagaaaagtctattcagagcctttgcccatttttaaaaattgggttatttgtctttttatgttgaattataagagttcattttacattctggatacaagacccttattaaatatatgatctgcaactattttctttctttttttttttttttttttgagacggagtctcgttctgtcacccaggctggagtgcagtggcgcaatctcggctcactgcaagctccgcctcccgggttcacgccattctcctgcctcagtctcccgagtagccgggactacaggcgcccgccaccacgcccggctaattttttttttttgtatttttagtagagacggggtttcaccatgttagccaggatggtctcgatctcctgacctcgtgatctgcccgcctcagcctcccaaagcgctgggattacaggcgtgagccaccgcgcccggctgatctgcaactattttctcccattctgtggatcgtcttttcccttgatggtatcatttgcagcacatttgtttttattttgatgtaatacagtttatctcctttttcttttgtcacctgtgcttttgatgtcccatctgagaaaccgttgcctaacccaaggtcacaaagatttactcctatgtttttctcctaagaattttgtagttttggcctggcgcggtggccaaaattacagttggccaccgcactccagcctgggtgacagagtgagactctgtctcaaaaaaaaaaaaaaaagaattttgtagtttcatctctgacatttaagcctgtggtccattttgagtttgtttctgtgtatgttgtgatataggagttcaacttcattagactctcagttctgtttcattgatctatgtttgtccttacgccagtaccacaatgtcttgagtactatagctttgtagtaagttttgaaatcaggaagtgtattagcccgttttcatacttatatgaagaaccgcctgagactgggtaatttataaaagaaagaggtttaactgactcacagttcagcatggctagggaggccttgggaaacctaacaaatcctggcggaaagcgaaggggaagcaaggcaccttcttcacaaggtggcagaaaggagaaggaacgcaggaggcactaccacacacttagaaaaccatcagatctcatgagaactcactcactatcacgagaacagcatggaggaaactgcccccgtgattcaattacctccacctggtctctcccttgacacatggggattatggggattacaattcaagatgagatctgggtggggacaacaaagcctaaccatatgaggaaggagttaactgtgagccagctcctgctgtggctaagggaagggaatcagaaggtggagagacttgaaattgcactcgagagatagtgccttccaactttgttcttctttttaaagactgttggctcttctgggttctttgctgttgcgtaagaattttaggatcagcttgttaatttgtgaaaaaagccagctgggatcttactagggattgcattgtatccccaaatgatttggggagaattgctatctccaggattgtgtcatcgcagagatagtcttgctgcttcttttccagtctgaatgccttttatttctttttcttgcctgattatcctgtaaaaaaaaaaaaaaaaaaaaaaaaaaagtgttacatagaaatggagagagcaggcatccttgtcttgttcctaatcttagggggaaagctaaggccccacctttgtcatcacttcagaggttcctggtgtcactcatgcttgagtgtcctggagttcccacagtgtgaatctggttgcttcttggctgtccctactgttggctcaaaggtcggccttcttgggctggtaaggccccactcagacctgtactgccaaattttcctactattatttcctctccctttttctttgttcctgtaggcacatggctttttgaaggtccttttagtagagtttgggctgggaaaaaaattgcatgcatctgttcaacccattatctttaaccacagtctcttgtttcatttggattgggacggctttcctgtggttatgatttggtgttaagaatggtgttactttttttgttgtcgtttattcggtgacttttaaacttagctgtgtcctaaaaggaaaagtctttccttctctaatgaattcttatgaatgagataccatgttcatggaacacacatgcatccacatgtgtaaacacaaacaatttcaaaaacattgctgcataggacagttgcatggaaacaaatggtgttcaagatgagtttcacttgccttttacctctgtgtgtatttgtctgtgaatcaattctagccaattttaggatgaaaaataaaactaatgctaatatagtgaatgtgtagagattttgaaaacccctgatcctttatcccaattgtaaacaatgttctttttagtacttctgtaataattgctatttctcttaaagccaaagagaaagtaacttttctatcttctgtgattttccagagtttcagattttgaaaagaaaacaagtaatcgttctgagattgaaacccagacagaggggagcacagagaaagagaatgatgaagagaaaggcccggagactgtgagtcctaagattccacggccactaccacacccacacacacgagagtagtccagccactgaattcaaatcttgtgatgggttatttgctttagaaatatagaaatcatgttgatattgaatattatctatctattccttttatatgtccttgtcctgctctgtgtcaattgtagcgagatgtatttcttttttgttgttgttgttggagatggagtctcactctgtcgccaggctggagtgcagtggcacgatctcagctcactgcaacctccgcctcccaggttcaagcagttctcctgcctcagcctcccaagtagctgggattacaggtgcccgtcaccacgcctggctaatttttgtatttttaatacagacagggtttcaccatgttggccaggatggtcttgatctcttgacctcgtgatcctcccacctcggcctcccagagtgctgggattacagatatgagccactgcgcccagctgcaagatgtatttctatcagtattctacaaaacgatttcctatgtctcttcttgactgatttcttctcctcggtccttcaatgaacaagcctactgtaggaaaaggaatgttgtccactttataatgagatcatttgaggatatgacttagaaacttgagggagaattgaaagatttgggttctatcccattactggtttgaataaagtagtttgaaaggaaaaggttcactgttgtcttgttcagtgttgtctggtaattgagagaggtgccttcgagtctgcagagagtcttcagctttcggaagttaatgaagccgtgaaggttgatagccataggggcccacgtgaaaggcatttacataaaatattcactttaggtaattaatttattcaacaaatatgtacattgaatgcctattgtatgtcaggagactgagaccttactgttgaaagaagagagaacatttagaaaacagatgaagagaccaccagtgaataatagttccctgttgactaaaacgaattcaacagccagtagcagggaaatatggtctttcaaggcatcagaaactcatttacaaaaattatagagctgccaggaaaaaggctgcacaacaaaaatagttgagtaaactagaaacatacactgggaagagagtatgggggcaagttgttagctggatagataggactgtgctttgacacctctgtggtctatgatctctgaacctggaatagggttcattttaatagcgataaagtcattatcccagtgcatccaaattgattagttcatgctttattaggaaacagaagttacccaaaacttagcaaacctaagtaccaagtatccaaaacattcttttcctacacaatgtttggggtattgtcaaagttggattgattcaccagccagtcttaattggctactaatggttcagcctgttttctcctaaagaggtttgtttaatgtcagatgataattgtacagatatgtttgggatttcccgtatgataggttggaagcgaagtggaagcactgaacctccaggtgacatctctgtttaaggagcttcaagaggctcatacaaaactcagcaaagctgagctaatgaagaagagacttcaagaaaagtaagaatgagagagcaattttatcctcctttgaaatatacatttttacaaagtatactactatataaaaacatagttttttaactatgttatgactaaaagaaaaatagacacctaattaaaatataaattcagaatatactaatgttccagttaatgtgtgagcatgaaatacttgtaagatggggggttggggactggagaactttaattctgccatttaggggcatttgttaaatgtacgagcctgggtaagatctctacagtaaagctgtgagctagttttcctgttactgacttaagctgatgacattgatgtgagtaagcataaagaaagatgaaaagagcataaagatcttgagtgacatttatttggaaaaaggtcaatttcaatttgttatttcaatcagttaattatttcaggctaacatgtagattgagcgtttggcatttgcttgtttctcttgatgtaagaagttacccaaaacttagcaaacctaagttgatgctgttctattggattcattggcaaacatgtttctagcacaatacggaggtgtgtgtgttttcttgaggttataattcccaacactctgtagaattatggtaacgtgatacaacatggcagctacaactaaggactttggacaaacagacctagatttaacatatgagtctgcaacttatttgtgttgggcaaggtatttatttcaattctctgagcctgtcttggcatttgtaaattgtgtgttgtaatttcttctatatacattgtgttaaatgatatgtctaatgggctgggcattaatgctttgtgcatagctgtcatttatttgtattatattgaaatcctctttccgatctttaagaagacttaggggaacttcctttttcccttattgaatctttgtcagaaactaaagtctttgcaattgacagaacctataactttttttttaatataaaagatatccacacatcactacatgagaagcgccttagctaattactactgtggtctgtgtttaaatactaaaaatgtatctgtatgactagtttaaacaattattcaaagaggacagtactgcatgtgagcttagatctgtacttttttatgtttaggcgtaagggttcagaaatatggccaggtctagtgaagaagcaaggaggattatgtatttcattttgcattcataaaccctacagccctaaaattcttatattgtacataaccttggggtttgtttaaaagccactgcgacgtaaaggagcattgtttatcctcatgaaatcttgacctttcttaggtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtgagtatgttgagtcagaagggcagcgacggggcagaggagggagaatcgcctttttatacagattggaattcggatttgagaataaattttaaaaaatttctttttcacttatctgaaggagtcctagcagacctctcagagagggggataaaatttaaaagttttgtcataataaaattatgctgattgtttgcactctgtcttgatttttcagaaaagattttttttgagagtaagaaatgctagtaggtcgtggggtgataaaggtaggcgagaagatttttctactggagtgttcagaaggttgggaggcaagactataagtttctatgatattttccccaggattccattttttaatatcttttttaataggtccaaattaactgtgctacagatgacacacaacaagcttcttcaagaacataataatgcattgaaaacaattgaggaactaacaagaaaagaggtattcactgaaaaaaattacttccatagcctagtaatgaacagaaactgttgaacgttttgtatataaaatagttacatgaatccttcactaaatctggtttcaaaggttgttttccaatgtatcattatttcttgcatctagggtttgtaacttctgatgttccacatatgtgtaatgtgctttattgcgtacaaagatgatgtgaatgtcctatggtcagggattaagcacttcgtatttctttttttttttttttgagacggagtctcgctctgtcgcccaggctggagtgcagtggcgcgatctcggctcactgcaagctccgcctcctgggttcacgccattctcctgcctcagcctcccgagtagctgggactacaggcgcccgccaccgcgcccggctaattttttgtatttttagtagagacggggtttcaccttgttagccaggatggtctcgatctcctgacctcgtgatccacccgcctcggcctcccaaagtgctgggattacaggcgtgagccaccgcgcccggccagcacttcgtatttctaaggataggtttgtaggagagctaagagcatgggcttctatgggtaggaagggccatctgctctggggaattgtgcaagaccagcgtgcctgctgtcagtgaatttgggaccctggaatcatcagcctgcagtttaaattcataataatggaccaggtgtggtagctcatgcatgtaatcccagcactttgggaggccaaggctggaggatcatttgaacccaggagtctgagaccagcctgggcaacagggaggccctatctctacaaaaaataaagagttaaccaagtgtgatgttcgtgcctgcggtcccagctccttgggaggctgaggcaggaggatcacttgagcctaggaggtcaaggctgcagtgagctgtgatcacgccactgtactccagcctgtgacagagtgagaacctgtctctaaaaaaagaaaacaataaataaattagtaataatgccagcatggtgtgatagtttagagaccacagaagcttgtgaattagaagggatctttgaatttttagccttgtaaatatactctttgtttttcatttattttcttttaaagaggaggttttgccatgttgcccagaatggttttgaactcctgagcttaggcaatccacgtgcctcagcctcccaaagtgctgggattacaggtgtgagccatcatgcccagcagtagtgttcctctcttggacctaataattttaaatttaaaacatgtttcttcttttccactgactgcaggaagtaacaagtggcaaaataacagtatcaacgagtcacagccttattaacattggagtttgttattgtatccctgatttcggtgttatcaccttttttttaggaattcattatttgcaagccacaacttaaatacaactttctgaataagttagcgttgctgattaatagactggttagagctgatacattttttagatctcgctatgttgcccaggcttgtctcccactcctgggctcaaacgatcctcccacctcagcctctcaattctaggcatgagccaccacacccggccagagctgataattaaaaaaataaacctttttctaatattttactaaaacaggcagaattatttcaaaaccatttctagaataaatgtttctttttcagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcaggtgaggcaccttccaaaaccccagctgagcgaggccagccctgactgtattctcgcattggaaagcaatggtgtttagaatgtttgtaattttctattttatatattttttcacccgtgagtgtattaaaactttaaaattgaaacatttggaaagtgctcagtggatcttatctgttctacatttaataggtaattggattctttccagtttgtggcattatgattaacgttgctaagacattcctgtgcatgttgctctgttcacatgtggatattttatatttctgttgggtacacacctaggagtggagtcgctggatcataggctctgcatgttactcacttttaacaggtaatgccaaacagttttccagagtggttggaccagttttcactcccatcaacagagagtttccatggctctacatcttaccaacacttctattatcagtcattttcctttaaccactctggagggtatatagtggtatctcatttaatttgcatttccctgatcactaatgggaaagagtactttttcaagtgtttttggcctttgaggtatcctcttttgtgaagtgccttatcaagcctgccttttttttttttttttttttttttttttttttggtttggtttgcctggcattttttttgagacagagtcttgctctgtcgcccaggctggagtacagtggtatgatctcggctcattgcaacctctgcctcctgggttcaggtgattctcctgcctcagcctcccatgtagctgggactacaggcatgtgccaacatgcctggctaatttttgtatttttagtagagatggggtttcatcatgttggccaggctggtctcaaactcctgacctcaggtgatccacccacttccacctcccagagtgctgggattacaggcatgagccactgcgcctggcctggctttttaaaaaaatgtaatgacttctatgtatactgcacatacaagtctttgtcagttatttttgccttttcactctcataaaggtgtcatttgaggaacaaaagttcttagttttaaggtagtccagtatatcagccttctcatttatgattagtactttttgtgtcctgtttaagaaacttttactaccccaaggccaggaagatattccctctgttgtcttctaaaaattttgttgtcttaccttttacattaagatgtacaaaccatttgaaattggcttttgtctatagaaaatgaggtaagggtaaatatttatttttttcctatatgaatatccagttaacttggcaccatttaagccatcttttcttaagtgctctgctgccctcttcatcataacttgtgtccatatatgcatgggtctgtttctggattttgttctattttattggtctactcatatatctttgctctggtaccatgctgtcttattataaagcatcaaactttttttttttttttgagacagagtctagctctgtcaccaggctggagtgcagtggcacgatcttggctcactgcaacctccacctcccgggttcaagccattctcctgcctcagcctcccaagtagctgggactacaggcgcacaccaccacgctcagctaatttttgtatttttagtagagatggagtttcaccacgttagccatggtggtctcgaacagttgacctcatgatctgcccagctgggattacgggcgtgagccaccgtgcctggccagcatccatttgaatggttggatcatgagttatgtccatttctcagttaaataaatattaccaagctgtcttctaaaactgcctaaccaaaattatactctcaccaaaagataagcactcctatgtcctcacattcttgcattattttaaaaaccttggccaatctgatgaacatattatttttaatataataatgtcagcagtttgttattccctacatacagactcattatcaccagctgcccagagtggctgacccctgtgaaatgcagccagttgactggtacttatagctttagttttatttctacaggaatagagcctcaacctgcattctcagctcttcagagcatttccttgcttgggtaactcatgtcattgggttcttccactctgctgcagtcatgtatttttgctcattctagacatagacatttgttttctcttgatggatactcttgctcattcattcaataacttcttttttgagcacttataatatgtcagagacggccgggcatggtggctcacacctgtaatcccagcactttgggaggccgaggcaggcagattacaaggtcaggagttcgagaccaacctggccaatatggtgaaaccccgtctctactaaaaatacaaaaattagccgagcgtgttggcgggcacctgtagtcccagctactcgggaggctgaggcaggagaattgcttgaacctgggaggcagaggttgcagtgagccgagatcatgccactgcactccagcctgggcgacagagcgagactctatctcaaaaaaaaaaaaaaaaaaaaaaaaaaaaatcagacactgttcttactgcctgggatataggaatagacaaaacaaagactctgttcttactggctcaaagctagtaagtgatagaagtgagactaaatcgaatttaagtcatactccaaaacacccatgatccactgccaacactgtgagatgctgaaccactcacattgataggatatcccttaaaatgatctctttattacatacttttaagtaactatagttttgatagaatatttagatttttaattttacttttaaaataatggataataatttatatctcagaaccagcaaatttgaaaagaaaaaactaattttaaacatagaaagtgaaaaattagtattaaattccaataccaatggaacagttcactcaattagctgacagcattaaatattaaaacactattaagtgttttgttatttataattttttaaaaattacttaatttttaaaatgttgtgttgttttgttttttgaggcagggtcacactcctgtcacccaggctggagtgcagtcgcatgatcgtggttcactgcaacctctatttcctgggctcaggtgatcctcgtgcctcagcctcccaagtagctggaactacaagtgcatgccatcatgcccagctgattttttgtatttttagtagcgatgaggttttgccatgttgcccaggctgtcagcaggaggcctcagttcctcaccatgtggtctcctccagagggatgcttgggtgccctcatgtcatggcagctggcttcccccaaagcaagtgatccaacagagagcaaggagtgacagcgtcttttgtgacctagtctcagaagccgtacaccatcacttctgcggcatcctggaggttacacaggttgggtttattcattgtgggagggaaacatacaaggtgatgaataccaagaggcaaggatcactggagccatcttaggaggctggtgaccacacatgggacatggattgaatatggaaaagttcatgggaaatggtcttctgtctggttcaaatgctgatttggccacttaacaaattttcccaagattaagagccgttaagtttgtaaaatgaggatagccattctttattctcaggatatttcataaggtaaaataagatagtaaatgtaaagcacccaacataggacctcacacatgtttggaatttaacaaatagcatctatttgtgatgattattcttttaaatttagcttaagaccagccttcataaatacacctggcagaatcaatttactatattaagtaatcatttactatattaagttgatcctgaattgtttattatctaaaagtccagataattttgctgaattaatggtacctacagtatttaaactacctatatcagtgcagttgcaggatttgtgttgtttaaagcacacacacaaacacagcttgtatctgctatcggaatgtacctggaaagtcatggtcattatactgttttctagcaggattgtgcatctgtgattcacaagggctattgaaggatacagcactacctcctcatcgcataaacactgtaagaatctgcattcatctaggtactaacttctgtatctttttttcctctaacagatggaagtttactgttctgattttcatgctgaaagagcagcgagagagaaaattcatgaggaaaaggagcaactggcattgcagctggcagttctgctgaaagagaatgatgctttcgaagacggaggcaggtaaggaaaagagagaggaggacccagagctcacatcagcatggccgtagaagaggtgcctgtccaaagacgttcctgatttgaactataagaatagctgtgttcgcgccactgcactcctgcctaggtgacagagcgagtcccctgtctgaaaaataaataataataataataattgcttcacttacacttcatgtgatcatgttcccaacacttagtttgtcttacaggaaagcttgacagagacttgtgggagcttgatcaagctccttgcttttagataagcaaggattttgatttgattttaaaatgttgtgttgttttgttttgttttttgaggcagggtctcactcctgtcacccaggctggagtgcagtggcatgatcatggttcactgcagcctcaacttcctgagctcaggtgatcctcgtgcctcagcctcccgagtagctggaactacaagtgcatgccaccatgcacttgtaacaataatgttacgtgtcccaatgacctatcttgccatcgtcacggatgaaatattcgtagcactttaaattcctgaatattctttaaaaaatactaacaaaaattacttctgacttttagaaatttattttgagaaagttttaaaacacagcaaaattcagagaataaaataacagactctactatgtactccttcctgcattgacaactgcttttatttgcttcaagtattttttttttattaaaggaaaaagggagttgtagttagaatcaaagtctcctttgttccccatccatcattatattcctttcttctttaccttgtgctgttaggaatttggtgggtagcttccccatctattttatacttttacatatcacatacacacttacctatatcatatctcaaaaccagataatattgatttctctgtgtttaagttacaaaatgatcactgtaggtattgttctgcagcttactttacataatattatgattttgagctctcttgatatgtgcggatgtaatttattatacttcattgctgtattttgatttataaatatgccacttctttctaatctgtttcctactgatgacagtttggttatttcctgattttttttaactgtaattatttactttcactagtctcctagtgccaatagtatttaaaactaaaattagtctggtttttatgaaccttggcagtgtagtttgagtcttttttcccctacttctgtggactgtctgctcagtgttgtcatgtttcggggttgtagaacatcacacagcgtgttgcttttcgtcctggcaggcagtccttgatggagatgcagagtcgtcatggggcgagaacaagtgactctgaccagcaggcttaccttgttcaaagaggtgagtcccgtgtgatcctggattttcaggaaatagctatcctatgaaaaagatgcttgaagaaaaattccacttcattctctacaatggattccaaatcaaggcaccaaaaatatagcacccgtcagtctcattaccacagcactcccatctccatccattacccaccgaatccagaccagacccttcaccctgccagaaggtgcctggcacggccacactttttcttttttttctttttttttgagacagaatttcgctgtgtcgtccaggctggagtgcagtggcgagatctcggctcactgcaacctccacttcctgtgttcaaacggttctccttccacagcctcccgagtggctggaattacaggcgtgcaccgccacacccagctaatttttgtatttttaatagagatggggtttcaccgtgttggccaggctggtctcgaactcctgacctcaactaacctgcctgtctcggtctcccaaagtaccgggattacaggcgcaagccactgtacccggcctacagccacacttttaaaccgtgtctccctctgttctcttacagacattagccagactgaatcatccccttgaacttgtcctaagcttgtatttgcttgtattatgcccttcacagagacgcccttttcttatgcatattcctgtcttcctccagtctctttccagtgacctcttaccccatctttaaaatctatttcaaactccatctccatgaactgtttccattttgaacttccaaagtactttattcctctatgttggtacctgtctgctattttataggtgtttgtgtgttctaggtagatacttaagtactttatttttatttctatttttattttgagacagggtcccactctcttgcccaggctggagtgcagtggcatgatcgtggatcactgcaacctccgcctcccgggttcaagcgattctcctggctcagcctcccaagtagctaggactacaggtgcacgccaccacgcctggctaatttattttagtagagacagggtttcaccatgttggccaggctggtctcaaattcctgaccccaggagatctgcccacgtcggcctcccaaagtgctgggattacaggcgtgagccaccacgcccggctaatttgtattttcagtagagacagcgtttcaccatgttggccaggctggtctcaaactcctgacctcaggtgacccacccacccaggagaggtctcctagaacagaatgtaccttctcagaagcagaggcaatggtctcatttattcagcagatacttttgagtcctccaggatgtgcaagaggtactgcttatgctgggagtcagagagcaaaggcagccccggatgctgacccctcttctctggcaatggggtgattagaaagctccctgctttggaaaccatgctttatgatcaatacatacctcttcagaaatgcttcttaaggctgctgaacacaatctcaaatattcatacatgttactacatccaagtaatgtacttagaagcaaataaaggttgcctccaaaaacaaacattttgagagccaaaccaaagtctaggctgagtccaaaaaaccaagacggtttgcattggtccagaaaggtcagaacagagagatgcccgggagccagaaataatttcttgcatccatcacagtttgtatggatccaggacctagctagggtcagggcttatttcgaacatgctctgattgaggcttgaacctcagtactactccaaaactgggggaaatgatagggtgcattcttccatcattcctccttgttaactttatgtagataactcctgttcattctcaaggcccctctcaagttaattggtcgcatctaggaggcctttctgcaccaagatctaaactaggggcccttccctttctcccatggacacccttgcacctaacactcacctggcctaacactcgtgcctaacacttttgccacgccactgctgccacatctctcacctggcttctccaccacgtctgtctcccggcacacagcagtgtctttatacatcccagaggattcaaaagtgtctgctgaacaaatgagatgattgcctctgcttctgggaaggtacactctgttctaggagacctctcctgggctctgtggatttctttctccacattcccttcctcttttgcttatatcctcattcatcccttttcttctcgcagatatcctagtatttactagaacatgcattgtcctgtacctagaactgaaaccatgtattttcgcataccttgaaactgggatacagtttaaatcctaaaagaatgcaccaatgcacctgtaatcccagtacatgggaggctgagatggaaggatcagttgagaccaggagttcagaaccagcctgggcaacatagtgagatcccatccctacaaaaattttaaaaactagccaggtgtggtggcacgtgcctgtagtcgcagctattcaggaggccgaggcaggagggtcactggatcccaggagttagaggctgcagtgagctatgattgtgccactgcactctagcctgggagaaagagcgagactccatctgtcaagaaaaaaaagtaaagaaaagaaaaaaatcctaaaacaggccaggcgcagtggctcatgccagtaatcctagcactttgggaggccaaggtgggcagatcatgaggtcaggagttcgagaccagtctggccaacatggcaaaaccacatctctactaaaaatacaaaaattagctgggcgtggtggcgcgcacctgtgatcccagctactcaggaggccaaagcaggaggatcacttgaacctgggaggcggaggttgcagtgagccaagatcgtgccactgccctccagcctgggtgacagcgagactccgtctcaaaaaaaaaaaaaaaaaaaaaaaatcctaaaataatagggaagcaggtatcacttggagagatttttctctatgtgcatcgtgatgacttcagttaaagaccaaacacctgtgctcatgtcccactacgtgttgaatacgaagttgaactgatgttaaaactcgccatctgttcttcaagtgaaacaaacacaactgcctgcaaaatggaactaatggaattatcatacttattcccaggagctgaggacagggactggcggcaacagcggaatattccgattcattcctgccccaagtgtggagaggttctgcctgacatagacacgttacagattcacgtgatggattgcatcatttaagtgttgatgtatcacctccccaaaactgttggtaaatgtcagattttttcctccaagagttgtgcttttgtgttatttgttttcactcaaatattttgcctcattattcttgttttaaaagaaagaaaacaggccgggcacagtggctcatgcctgtaatcccagcactttgggaggtcgaggtgggtggatcacttggggtcagggtttgagaccagcctggccaacatggcggaaccctgtctctaccaaaattacaaaaattagccgagcatggtggcgcatgcctgtagtcgcagctactcgcgaggttgaggcaggagaattgcttgaacccaggaagtggcagttgcagtgagccgagacgacaccactgcactccagcctgggtgacagagggagactctgtctcgaaagaaagaaagaaaaaaaggaaggaaggagaaggaaggaaggagaagaaaaggtacctgttctacgtagaacacctttggtggagttccatcaactcgcaaagtagaatccttacctactactcttctgataataattttaatattttttatgtttggttgatgcgagcagctgcactgctcatgcagttagctagcatgtgacatcatgtgacaaagttcatgtaattagatggaagaaacctcactgattaattttaagaaccttttagggatgcaggaacaatgaagtggccacagtatgtgctgtttttgaagcatttttaaaaacgaattgtagttgtttttcttcatttaaaatggatctgttggaggttatgtgtgtatgttgtagttttattgcagccacaataattttaccaaagttttcacataggcagttagcctttacttaatatcaagacaagtgaaaaaatattggcatcgatgaaaccgataacattggcctcattggatttctttacccattcacagtgtaaagaagttaccttcatgctttcattgtacctgcaggcctgtgggcttgtacagtagataattaatttctaaaaagaacagctgcctattttcttcctaggttaggttatatcttcataatcacaagaattagtgatggcaaaataaaattttgcttatgaatcttttacattgtttatatatgattaatatcatcatatatattttctgtattaagctcatttggcttcatttaagctgtatacttagtcatatatctttcattagttctatggatatgagcagatccctttactggagcccagtatgtgctgtgtgagttagaagtcattcttgctgagaaggtgaataggtagggatttgccttgttttgtaagtctacaatttgccaagagtaaataacactggaccagctgtaaaagtaaacagtgtgtttatgcattgagatactaaagcatttaagaaaaaattaaaagatctcttttgtttaaatttgtcttaagaatctctcctagacaaacttgactatacacacacacacttcaataagaatcaaaatagttcattatatcttcatgccaagagaacacatgatacaaattcaacagtaatttcaattaaacgagtcccctaccacacctcagggagaatatttgttaaagacatttctaaagtaactctgacatttttacttttctttgttatggtttctaatgtaaattttttcccttatcatcttatactggaggaaatatatttctatagtctcctaaacagagaaggaaaccaaatggtttcacatttaaagttattggacataatccttttactttgttcaagtaaaaatattcagcaaagccagactgaaataataacatgaatgggcttattaagcttgtgaccaaatcgtttgtttaatagttggaagaattacaaatgaaggccatttcacgtagtcagtttaaattaaggctgttgtttccagcttggtaaatagcttataaactctgtgcacatcttacctcatttatttattctaatatcccttcaaaggctctgttccatgtgaccagttaactatacctcctaggaattttgtgtctgtaaaaatacatagttgaagattgcactcttaacaggtttacgactatcctggatagtattgttaactgtgtgcacattgttgtagagcagatctctagaactttttcatcctgcgtgaatgaaactctacacccattcaactgcagcccccattctcccctccccagtccccggcaaccaccactctcctttctgcttctgagtttcacatacctcctataagtgggattacatagtatttttccttctgtaactggcttatttcatttaacatacctccctcattcaaaccataacttcctggatgaaggtttcaggcaaaagcacctgggaaaccaggcctttgctactcatttaccccccattccccaccccaccccccgccccagtcccgtgcctccacctgctgagcaccgttccatttgccgatgctctcatgttaccctgggagacataaggtagaagagccatggggactctgctcgagggtggtataaggcatttgaaactaagcctcttcattaacccaatccatatcacagagaggcagcatgggtgcccctagcccaatggccgggctatttgctactgcaataataggtttcttataaaccgtcagcctcctgagtagctggcaccacagggacaccacaggctattttttaaaaaaatttatgtagaaatggattctcactgtgtttctcaggctagtctcaaatttctaggctcaagcagtcctcccgcctcagcctcccaaagtgctaggattattggtgtgagaggaatccctttcaacccttgaaaataaatcaagcggtgatgaactccctgagcttatatttgcctaggaaggtctttatttctctttcatttttgaaggacagtttggctggacatagtatgtcttatttggcagtttgtccccttctgcactgtgaatatattatgtcagtcccttctggccatctggcaaggtttctgctatgaaatccactgttttatgaaggatctcttgttcatgacaagttgctttttgattttaaaatgactcaatgtggatgtctttgggttcattctagttggagtcctttggaattctcgaatctggatgtctattttctaccccagatttgggaaattttgagccattattttttttaaagaagctttctgtccctttttgtatttcttcttaaattcccatagtgtttatattggtccacttaatggtgtcccagaagttccttagactttcatcattttaaaattattttccctttttgatccactgactggataatttcaaataacctctgagttcattctttcttctgctttatcaaatctgctgttgaacccctctaataaatttttcaatttattttattcttcactgccaacatttctgtttggttcttttttatactctctctgttgatattctcattttgttcatacatcgttttcctgagctcactgaacatcttttgaattttttttcaggtaagtcatatacctctagttcttggggtcagtctctagagatttcatttgcctctttgggccatattttcatatgtttcttcatgtgtcttgtgattttgtgtcgggatccatgtatttgaaaaacaactctgtcttccagtctttaaggactggcttcatacagggaaagatcttttccaatcatcttggctagagattctgagatctcccagacctcttccatagatacatccccccagacctgtgcatgcaaatgttcaattagatttgctagtctcgtttttcacaatctgcagcctcttgttccttccagtgtctgcctgcagccctgcatattccctggagctgccacaagccatccagcactccttatcttattctcagcagcctctaggtctctctctagagcattctgggttctgtcaaaactctttaagttgggcgagatagaaccagttccctgggcagccccttaaaagccagaacatttgaaacacactccactagtctctttccctccacaagggagaggctggctgagctgtattaacctctgtatgctgcaccatgagtcctggagcagtagcaggtcaggtcacccagctctctctctttcccagtgtcctccaggcatctagagtatgctgtgttccatcagcactccaagacaaagcagaaacgagtccctcagacagcctcccaaaagggcagaatgttggacacactttgctcttctcttttcccctgtataggagaggccgccaagatgtattggcctctgttggactgcagatcctctacagcagcagcaagctgccatgcttttgttctcagtagtccccaggcatctatggtatatcaagcctttcaatgctcaagacaagagaaacagtcccctgggaagccccctgaaaaactggaaattggattcaggctccaactctctccctccccagggaaaggcaagagccaggtgttttctcccacttgtttcacactgagctggggcaatgggtatggtgacagagtacatgccaatcccagtctcctcttttgttctgagtggtccctagactgatacccttttcagtcagttcctaaattcaggcaagagagaaactaatccctcaggcagccccctgaaaagtctgcacattgggcataggtattagtcttctctttgcctccccagggagaggtcaggagctgggagctttctcctgattgtgccacactgagccacaggaggtactatggcaagggtatgccacaggttctcctacatggctggtttcatgcccatctggagtggaggagcctgttaactattttcttgacttctcacaaagggaattcatccatgtattgttgaaccagtgccttcccggcggggaaggagggcctggggcttcctgttctgccatctcccttttgtaaatgctgccatgctgtatacttttttttttttaaaggataaacatttctgtttaaaggataaacattcaacgttaactggaaatgaaaaggagacgatttttagtctgatacttataatgcaatattatttgcaattctgtataaatagatttcagaaacttcgatttcaaatcccaattaaattaaagagaggaaaattactgagaggaactgcagttccaaatttttcctttcagggcagtca

SEQ ID NO: 3 - DNA, 15 bp, Homo sapiens
  Putative OCTB/TST1.01 motif
cagcaattccacttc

SEQ ID NO: 4 - DNA, 22 bp, Homo sapiens
  Putative AP1F/TCF11MAFG.01 motif
atgatatgacccagcaattcca

SEQ ID NO: 5 - DNA, 11 bp, Homo sapiens
  Putative GATA/GATA.01 motif
tgatatgaccc

SEQ ID NO: 6 - DNA, 11 bp, Homo sapiens
  Putative EVI1/EVI1.05 motif
agttatgatat

SEQ ID NO: 7 - DNA, 16 bp, Homo sapiens
  Putative FKHD/FREAC2.01 motif
gaaagttaaacagaga

SEQ ID NO: 8 - DNA, 13 bp, Homo sapiens
  Putative IRFF/IRF1.01 motif
ggaaagttaaaca

SEQ ID NO: 9 - DNA, 11 bp, Homo sapiens
  Putative MYT1/MYT1.02 motif
ggaaagttaaa

SEQ ID NO: 10 - DNA, 18 bp, Homo sapiens
  Putative XBBF/M1F1.01 motif
gagttccttggaaagtta

SEQ ID NO: 11 - DNA, 12 bp, Homo sapiens
  Putative NFAT/NFAT.01 motif
ccttggaaagtt

SEQ ID NO: 12 - DNA, 13 bp, Homo sapiens
  Putative IKRS/IK3.01 motif
tcctcggaatatt

SEQ ID NO: 13 - DNA, 15 bp, Homo sapiens
  Putative OCTP/OCT1P.01 motif
ccaaatattccgagg

SEQ ID NO: 14 - DNA, 12 bp, Homo sapiens
  Putative PCAT/CAAT.01 motif
tggaaccagtga

SEQ ID NO: 15 - DNA, 9 bp, Homo sapiens
  Putative AP1F/AP1.01 motif
ttgattcag

SEQ ID NO: 16 - DNA, 15 bp, Homo sapiens
  Putative BARB/BARBIE.01 motif
aactaaagctgagac

SEQ ID NO: 17 - DNA, 20 bp, Homo sapiens
  Putative PERO/PPARA.01 motif
taaagctgagacaaagtcca

SEQ ID NO: 18 - DNA, 11 bp, Homo sapiens
  Putative  AP1F/NFE2.01 motif
ttgtctcagct

SEQ ID NO: 19 - DNA, 14 bp, Homo sapiens
  Putative HNF4/HNF4.01 motif
gagacaaagtccag

SEQ ID NO: 20 - DNA, 8 bp, Homo sapiens
  Putative SMAD/SMAD3.01 motif
gtctggac

SEQ ID NO: 21 - DNA, 13 bp, Homo sapiens
  Putative RORA/RORA1.01 motif
agaccaaggtcaa

SEQ ID NO: 22 - DNA, 9 bp, Homo sapiens
  Putative SF1F/SF1.01 motif
ccaaggtca

SEQ ID NO: 23 - DNA, 16 bp, Homo sapiens
  Putative AP4R/TAL1ALPHAE47.01 motif
tagggcagatgattca

SEQ ID NO: 24 - DNA, 18 bp, Homo sapiens
  Putative AP1F/AP1.01 motif
atgaatcatatgaatcat

SEQ ID NO: 25 - DNA, 10 bp, Homo sapiens
  Putative PIT1/PIT1.01 motif
attcatgcag

SEQ ID NO: 26 - DNA, 21 bp, Homo sapiens
  Putative MINI/MUSCLE_INI.03 motif
tgcagcgaccacaccagtggc

SEQ ID NO: 27 - DNA, 6 bp, Homo sapiens
  Putative HAML/AML1.01 motif
tgtggt

SEQ ID NO: 28 - DNA, 16 bp, Homo sapiens
  Putative OAZF/ROAZ.01 motif
ctgcagcaaagggtgt

SEQ ID NO: 29 - DNA, 8 bp, Homo sapiens
  Putative MZF1/MZF1.01 motif
gttgggga

SEQ ID NO: 30 - DNA, 15 bp, Homo sapiens
  Putative ETSF/ETS1.01 motif
ccaggaactggtttc

SEQ ID NO: 31 - DNA, 10 bp, Homo sapiens
  Putative RPOA/DTYPEPA.01 motif
tccatgaaac

SEQ ID NO: 32 - DNA, 9 bp, Homo sapiens
  Putative STAT/STAT.01 motif
ttcatggaa

SEQ ID NO: 33 - DNA, 12 bp, Homo sapiens
  Putative MYT1/MYT1.01 motif
aaaaattgtctt

SEQ ID NO: 34 - DNA, 12 bp, Homo sapiens
  Putative NFAT/NFAT.01 motif
ccatggaaaaat

SEQ ID NO: 35 - DNA, 15 bp, Homo sapiens
  Putative SRFF/SRF.03 motif
accatccatggaaaa

SEQ ID NO: 36 - DNA, 10 bp, Homo sapiens
  Putative CLOX/CDPCR3HD.01 motif
catggatggt

SEQ ID NO: 37 - DNA, 21 bp, Homo sapiens
  Putative MINI/MUSCLE_INI.03 motif
ccacccccccacccaccacca

SEQ ID NO: 38 - DNA, 14 bp, Homo sapiens
  Putative RREB/RREB1.01 motif
ccccacccaccacc

SEQ ID NO: 39 - DNA, 13 bp, Homo sapiens
  Putative SP1F/SP1.01 motif
ggtgggtgggggg

SEQ ID NO: 40 - DNA, 13 bp, Homo sapiens
  Putative EGRF/WT1.01 motif
gggtgggggggtg

SEQ ID NO: 41 - DNA, 14 bp, Homo sapiens
  Putative RREB/RREB1.01 motif
tcccaaaaccaccc

SEQ ID NO: 42 - DNA, 19 bp, Homo sapiens
  Putative SEF1/SEF1.01 motif
tgcctgatgatctgaggtg

SEQ ID NO: 43 - DNA, 21 bp, Homo sapiens
  Putative PAX6/PAX6.01 motif
gatcatcaggcattagagtct

SEQ ID NO: 44 - DNA, 19 bp, Homo sapiens
  Putative PDX1/PDX1.01 motif
atgagactctaatgcctga

SEQ ID NO: 45 - DNA, 16 bp, Homo sapiens
  Putative AHRR/AHRARNT.01 motif
tctaggttgcgtgctt

SEQ ID NO: 46 - DNA, 14 bp, Homo sapiens
  Putative FKHD/XFD3.01 motif
attgtcaacagaac

SEQ ID NO: 47 - DNA, 14 bp, Homo sapiens
  Putative SORY/SOX9.01 motif
tgttgacaataggg

SEQ ID NO: 48 - DNA, 15 bp, Homo sapiens
  Putative CREB/TAXCREB.01 motif
tagggttcacgctcc

SEQ ID NO: 49 - DNA, 21 bp, Homo sapiens
  Putative PAX6/PAX6.01 motif
agggttcacgctcctatgaaa

SEQ ID NO: 50 - DNA, 13 bp, Homo sapiens
  Putative E2FF/E2F.03 motif
gagcgtgaaccct

SEQ ID NO: 51 - DNA, 16 bp, Homo sapiens
  Putative AHRR/AHRARNT.01 motif
tcataggagcgtgaac

SEQ ID NO: 52 - DNA, 14 bp, Homo sapiens
  Putative OCT1/OCT1.05 motif
ctgcattagatttt

SEQ ID NO: 53 - DNA, 18 bp, Homo sapiens
  Putative AP4R/AP4.03 motif
taatgcagctgctgatct

SEQ ID NO: 54 - DNA, 12 bp, Homo sapiens
  Putative MYOD/MYF5.01 motif
atgcagctgctg

SEQ ID NO: 55 - DNA, 14 bp, Homo sapiens
  Putative SP1F/GC.01 motif
aagaggcggagctt

SEQ ID NO: 56 - DNA, 13 bp, Homo sapiens
  Putative EGRF/WT1.01 motif
gggtgggtgagca

SEQ ID NO: 57 - DNA, 9 bp, Homo sapiens
  Putative VMYB/VMYB.02 motif
agcaacggg

SEQ ID NO: 58 - DNA, 20 bp, Homo sapiens
  Putative PERO/PPARA.01 motif
tcctgagaggccacaggcca

SEQ ID NO: 59 - DNA, 14 bp, Homo sapiens
  Putative HNF4/HNF4.01 motif
aggccacaggccag

SEQ ID NO: 60 - DNA, 16 bp, Homo sapiens
  Putative B2TF/E2.01 motif
aaaccccgggtggtga

SEQ ID NO: 61 - DNA, 14 bp, Homo sapiens
  Putative RREB/RREB1.01 motif
ccccaaaccccggg

SEQ ID NO: 62 - DNA, 14 bp, Homo sapiens
  Putative GKLF/GKLF.01 motif
caataaagcagggg

SEQ ID NO: 63 - DNA, 12 bp, Homo sapiens
  Putative CLOX/CDP.01 motif
ccaataaagcag

SEQ ID NO: 64 - DNA, 8 bp, Homo sapiens
  Putative RPOA/LPOLYA.01 motif
caataaag

SEQ ID NO: 65 - DNA, 30 bp, Homo sapiens
  Putative HOXF/HOX1-3.01 motif
tttattggacataattattaggtcgtgttc

SEQ ID NO: 66 - DNA, 11 bp, Homo sapiens
  Putative ECAT/NFY.02 motif
tgtccaataaa

SEQ ID NO: 67 - DNA, 12 bp, Homo sapiens
  Putative PCAT/CAAT.01 motif
tatgtccaataa

SEQ ID NO: 68 - DNA, 16 bp, Homo sapiens
  Putative HMYO/S8.01 motif
tggacataattattag

SEQ ID NO: 69 - DNA, 8 bp, Homo sapiens
  Putative NKHX/NKX25.02 motif
cataatta

SEQ ID NO: 70 - DNA, 26 bp, Homo sapiens
  Putative GREF/PRE.01 motif
atattattaggtcgtgttctttttgg

SEQ ID NO: 71 - DNA, 16 bp, Homo sapiens
  Putative MEF2/MEF2.01 motif
caccaaaaagaacacg

SEQ ID NO: 72 - DNA, 8 bp, Homo sapiens
  Putative EBOX/USF.02 motif
ccacatgc

SEQ ID NO: 73 - DNA, 19 bp, Homo sapiens
  Putative CDXF/CDX2.01 motif
ggtgaattttatggcatgt

SEQ ID NO: 74 - DNA, 18 bp, Homo sapiens
  Putative MEF2/AMEF2.01 motif
tgccataaaattcacccc

SEQ ID NO: 75 - DNA, 10 bp, Homo sapiens
  Putative RPOA/DTYPEPA.01 motif
gccataaaat

SEQ ID NO: 76 - DNA, 10 bp, Homo sapiens
  Putative TBPF/TATA.02 motif
gccataaaat

SEQ ID NO: 77 - DNA, 11 bp, Homo sapiens
  Putative EBOX/SREBP1.02 motif
attcaccccat

SEQ ID NO: 78 - DNA, 10 bp, Homo sapiens
  Putative PIT1/PIT1.01 motif
aatcatacat

SEQ ID NO: 79 - DNA, 9 bp, Homo sapiens
  Putative AP1F/AP1.01 motif
atgaatcat

SEQ ID NO: 80 - DNA, 16 bp, Homo sapiens
  Putative HMYO/S8.01 motif
ggctttcaattacact

SEQ ID NO: 81 - DNA, 15 bp, Homo sapiens
  Putative OCTB/TST1.01 motif
tttcaattacactta

SEQ ID NO: 82 - DNA, 13 bp, Homo sapiens
  Putative NKXH/NKX31.01 motif
ttttaagtgtaat

SEQ ID NO: 83 - DNA, 10 bp, Homo sapiens
  Putative TBPF/ATATA.01 motif
ctttttaagt

SEQ ID NO: 84 - DNA, 11 bp, Homo sapiens
  Putative MYT1/MYT1.01 motif
aaaaagttgta

SEQ ID NO: 85 - DNA, 19 bp, Homo sapiens
  Putative CDXF/CDX2.01 motif
tgatggttttacaactttt

SEQ ID NO: 86 - DNA, 30 bp, Homo sapiens
  Putative HOXF/HOX1-3.01 motif
ttgtaaaaccatcattacaattcaaattta

SEQ ID NO: 87 - DNA, 19 bp, Homo sapiens
  Putative PDX1/PDX1.01 motif
gtaaaaccatcattacaat

SEQ ID NO: 88 - DNA, 10 bp, Homo sapiens
  Putative SORY/SOX5.01 motif
attacaattc

SEQ ID NO: 89 - DNA, 15 bp, Homo sapiens
  Putative RPOA/APOLYA.01 motif
actaaatttgaattg

SEQ ID NO: 90 - DNA, 12 bp, Homo sapiens
  Putative MYT1/MYT1.01 motif
taaatttgaatt

SEQ ID NO: 91 - DNA, 10 bp, Homo sapiens
  Putative OCT1/OCT1.02 motif
gatggaaata

SEQ ID NO: 92 - DNA, 14 bp, Homo sapiens
  Putative RREB/RREB1.01 motif
ccccaaaaatcccc

SEQ ID NO: 93 - DNA, 8 bp, Homo sapiens
  Putative MZF1/MZF1.01 motif
cgagggga

SEQ ID NO: 94 - DNA, 9 bp, Homo sapiens
  Putative PCAT/ACAAT.01 motif
cccccaatt

SEQ ID NO: 95 - DNA, 21 bp, Homo sapiens
  Putative STAT/STAT3.01 motif
cccaatttcaggcaactactg

SEQ ID NO: 96 - DNA, 24 bp, Homo sapiens
  Putative GFI1/GFI1.01 motif
aagacagaaatcagaccagtagtt

SEQ ID NO: 97 - DNA, 15 bp, Homo sapiens
  Putative 1RFF/ISRE.01 motif
cagaaaaggaaagta

SEQ ID NO: 98 - DNA, 12 bp, Homo sapiens
  Putative NFAT/NFAT.01 motif
aaaaggaaagta

SEQ ID NO: 99 - DNA, 14 bp, Homo sapiens
  Putative SRFF/SRF.02 motif
gtccagaaaaggaa

SEQ ID NO: 100 - DNA, 10 bp, Homo sapiens
  Putative RPOA/DTYPEPA.01 motif
tacattaaat

SEQ ID NO: 101 - DNA, 15 bp, Homo sapiens
  Putative OCTP/OCT1P.01 motif
ctccatatacattaa

SEQ ID NO: 102 - DNA, 22 bp, Homo sapiens
  Putative XSEC/STAF.01 motif
gctaccccagatgccaaagact

SEQ ID NO: 103 - DNA, 16 bp, Homo sapiens
  Putative LYMF/TH1E47.01 motif
tttggcatctggggta

SEQ ID NO: 104 - DNA, 30 bp, Homo sapiens
  Putative HOXF/HOX1-3.01 motif
agcaagtacgaatattagtctaccacctca

SEQ ID NO: 105 - DNA, 15 bp, Homo sapiens
  Putative OCTP/OCT1P.01 motif
actaatattcgtact

SEQ ID NO: 106 - DNA, 19 bp, Homo sapiens
  Putative SEF1/SEF1.01 motif
tttatgtgcatctgaggtg

SEQ ID NO: 107 - DNA, 19 bp, Homo sapiens
  Putative CDXF/CDX2.01 motif
taatatttttatgtgcatc

SEQ ID NO: 108 - DNA, 14 bp, Homo sapiens
  Putative OCT1/OCT1.05 motif
aatatttttatgtg

SEQ ID NO: 109 - DNA, 14 bp, Homo sapiens
  Putative OCT1/OCT1.05 motif
aaatattacatatc

SEQ ID NO: 110 - DNA, 12 bp, Homo sapiens
  Putative CREB/E4BP4.01 motif
agatatgtaata

SEQ ID NO: 111 - DNA, 14 bp, Homo sapiens
  Putative GATA/GATA.01 motif
agatatgtaataat

SEQ ID NO: 112 - DNA, 10 bp, Homo sapiens
  Putative VBPF/VBP.01 motif
attacatatc

SEQ ID NO: 113 - DNA, 15 bp, Homo sapiens
  Putative EVI1/EVI1.03 motif
agaaaagaaaagata

SEQ ID NO: 114 - DNA, 12 bp, Homo sapiens
  Putative NFAT/NFAT.01 motif
ggaaggaaaaga

SEQ ID NO: 115 - DNA, 15 bp, Homo sapiens
  Putative ETSF/ETS1.01 motif
gaaggaagtagagag

SEQ ID NO: 116 - DNA, 20 bp, Homo sapiens
  Putative YY1F/YY1.01 motif
gtggcaccatcttggctcag

SEQ ID NO: 117 - DNA, 18 bp, Homo sapiens
  Putative MYOF/NF1.01 motif
tcttggctcagcgcaacc

SEQ ID NO: 118 - DNA, 17 bp, Homo sapiens
  Putative XBBF/RFX1.01 motif
ttggctcagcgcaacct

SEQ ID NO: 119 - DNA, 11 bp, Homo sapiens
  Putative AP1F/NFE2.01 motif
ttggctcagcg

SEQ ID NO: 120 - DNA, 24 bp, Homo sapiens
  Putative BRAC/BRACH.01 motif
agcctctcaagtagctgagattac

SEQ ID NO: 121 - DNA, 14 bp, Homo sapiens
  Putative TTFF/TTF1.01 motif
cctctcaagtagct

SEQ ID NO: 122 - DNA, 28 bp, Homo sapiens
  Putative AP1F/BEL1.01 motif
tggtgcgtgcctgtaatctcagctactt

SEQ ID NO: 123 - DNA, 10 bp, Homo sapiens
  Putative GATA/GATA3.01 motif
tgagattaca

SEQ ID NO: 124 - DNA, 16 bp, Homo sapiens
  Putative AHRR/AHRARNT.01 motif
gtagtggtgcgtgcct

SEQ ID NO: 125 - DNA, 16 bp, Homo sapiens
  Putative MEF2/HMEF2.01 motif
atataaaaattagcca

SEQ ID NO: 126 - DNA, 17 bp, Homo sapiens
  Putative HNF1/HNF1.02 motif
ggctaatttttatattt

SEQ ID NO: 127 - DNA, 15 bp, Homo sapiens
  Putative TBPF/TATA.01 motif
atataaaaattagcc

SEQ ID NO: 128 - DNA, 14 bp, Homo sapiens
  Putative FKHD/XFD2.01 motif
aatataaaaattag

SEQ ID NO: 129 - DNA, 14 bp, Homo sapiens
  Putative OCT1/OCT1.05 motif
ctaatttttatatt

SEQ ID NO: 130 - DNA, 17 bp, Homo sapiens
  Putative MEF2/RSRFC4.02 motif
ctactaaaaatataaaa

SEQ ID NO: 131 - DNA, 9 bp, Homo sapiens
  Putative GATA/LMO2COM.02 motif
gagataggg

SEQ ID NO: 132 - DNA, 9 bp, Homo sapiens
  Putative AREB/AREB6.04 motif
gggtttcac

SEQ ID NO: 133 - DNA, 10 bp, Homo sapiens
  Putative CREB/HLF.01 motif
gtttcaccat

SEQ ID NO: 134 - DNA, 16 bp, Homo sapiens
  Putative ARP1/ARP1.01 motif
tgaactcctgacctca

SEQ ID NO: 135 - DNA, 16 bp, Homo sapiens
  Putative T3RH/T3R.01 motif
gtttgaggtcaggagt

SEQ ID NO: 136 - DNA, 10 bp, Homo sapiens
  Putative RARF/RAR.01 motif
aggtcaggag

SEQ ID NO: 137 - DNA, 13 bp, Homo sapiens
  Putative RORA/RORA1.01 motif
cgtttgaggtcag

SEQ ID NO: 138 - DNA, 8 bp, Homo sapiens
  Putative CREB/CREBP1CJUN.01 motif
tgacctca

SEQ ID NO: 139 - DNA, 9 bp, Homo sapiens
  Putative LYMF/LYF1.01 motif
tttgggagg

SEQ ID NO: 140 - DNA, 32 bp, Homo sapiens
  Putative HOBO/HOGNESS.01 motif
ggcggtggctcacgcctgtaatcccagcactt

SEQ ID NO: 141 - DNA, 12 bp, Homo sapiens
  Putative IKRS/IK2.01 motif
tgctgggattac

SEQ ID NO: 142 - DNA, 15 bp, Homo sapiens
  Putative CREB/TAXCREB.01 motif
ggtggctcacgcctg

SEQ ID NO: 143 - DNA, 13 bp, Homo sapiens
  Putative SP1F/SP1.01 motif
ccagggcggtggc

SEQ ID NO: 144 - DNA, 16 bp, Homo sapiens
  Putative FKHD/FREAC2.01 motif
agaaagtaaagaggcc

SEQ ID NO: 145 - DNA, 17 bp, Homo sapiens
  Putative TBPF/MTATA.01 motif
ttctttaaacccagttc

SEQ ID NO: 146 - DNA, 10 bp, Homo sapiens
  Putative MEF2/MEF2.05 motif
ggtttaaaga

SEQ ID NO: 147 - DNA, 18 bp, Homo sapiens
  Putative XBBF/MIFI.01 motif
ggggtgtacggaaaccta

SEQ ID NO: 148 - DNA, 9 bp, Homo sapiens
  Putative AREB/AREB6.04 motif
aggtttccg

SEQ ID NO: 149 - DNA, 8 bp, Homo sapiens
  Putative E2FF/E2F.02 motif
gcccgaaa

SEQ ID NO: 150 - DNA, 16 bp, Homo sapiens
  Putative LYMF/TH1E47.01 motif
actggggtctggagag

SEQ ID NO: 151 - DNA, 8 bp, Homo sapiens
  Putative MZF1/MFZF1.01 motif
agagggga

SEQ ID NO: 152 - DNA, 10 bp, Homo sapiens
  Putative OCT1/OCT1.02 motif
catgcaaaac

SEQ ID NO: 153 - DNA, 24 bp, Homo sapiens
  Putative PAX5/PAX9.01 motif
ggtacccattgaagtaagggccat

SEQ ID NO: 154 - DNA, 10 bp, Homo sapiens
  Putative RPOA/DTYPEPA.01 motif
cccattgaag

SEQ ID NO: 155 - DNA, 10 bp, Homo sapiens
  Putative VBPF/VBP.01 motif
cttacttcaa

SEQ ID NO: 156 - DNA, 8 bp, Homo sapiens
  Putative CREB/CREBP1.01 motif
ttacttca

SEQ ID NO: 157 - DNA, 8 bp, Homo sapiens
  Putative RPOA/LPOLYA.01 motif
aaataaat

SEQ ID NO: 158 - DNA, 17 bp, Homo sapiens
  Putative XBBF/RFX1.01 motif
tttcagcccagcaacat

SEQ ID NO: 159 - DNA, 30 bp, Homo sapiens
  Putative HOXF/HOX1-3.01 motif
cactgataccctcattatcaaatggttctt

SEQ ID NO: 160 - DNA, 13 bp, Homo sapiens
  Putative GATA/GATA1.03 motif
atttgataatgag

SEQ ID NO: 161 - DNA, 13 bp, Homo sapiens
  Putative IKRS/IK3.01 motif
tctagggaacagt

SEQ ID NO: 162 - DNA, 12 bp, Homo sapiens
  Putative NFAT/NFAT.01 motif
cattggaaacag

SEQ ID NO: 163 - DNA, 9 bp, Homo sapiens
  Putative AREB/AREB6.04 motif
ctgtttcca

SEQ ID NO: 164 - DNA, 11 bp, Homo sapiens
  Putative ECAT/NFY.02 motif
tttccaatgac

SEQ ID NO: 165 - DNA, 18 bp, Homo sapiens
  Putative CEBP/CEBP.02 motif
ggactttgggaacctccc

SEQ ID NO: 166 - DNA, 10 bp, Homo sapiens
  Putative NFKB/CREL.01 motif
gggaggttcc

SEQ ID NO: 167 - DNA, 12 bp, Homo sapiens
  Putative IKRS/IK2.01 motif
ctttgggaacct

SEQ ID NO: 168 - DNA, 22 bp, Homo sapiens
  Putative XSEC/STAF.01 motif
ggttcccaaagtccagtaggtg

SEQ ID NO: 169 - DNA, 8 bp, Homo sapiens
  Putative SMAD/SMAD3.01 motif
gtctgggt

SEQ ID NO: 170 - DNA, 11 bp, Homo sapiens
  Putative CP2F/CP2.01 motif
gcagcacccag

SEQ ID NO: 171 - DNA, 21 bp, Homo sapiens
  Putative PAX6/PAX6.01 motif
aggactcaagcctcagtccct

SEQ ID NO: 172 - DNA, 16 bp, Homo sapiens
  Putative ARP1/ARP1.01 motif
tgagtccttgatgctc

SEQ ID NO: 173 - DNA, 9 bp, Homo sapiens
  Putative RPAD/PADS.01 motif
ggtggtctt

SEQ ID NO: 174 - DNA, 16 bp, Homo sapiens
  Putative ECAT/NFY.01 motif
tcctcccaatctgggg

SEQ ID NO: 175 - DNA, 14 bp, Homo sapiens
  Putative SRFF/SRF.02 motif
ccccagattgggag

SEQ ID NO: 176 - DNA, 13 bp, Homo sapiens
  Putative SP1F/SP1.01 motif
tgggggcggggga

SEQ ID NO: 177 - DNA, 12 bp, Homo sapiens
  Putative EGRF/EGR1.01 motif
gggcgggggagt

SEQ ID NO: 178 - DNA, 11 bp, Homo sapiens
  Putative AP1F/AP1.03 motif
agtgactcccc

SEQ ID NO: 179 - DNA, 18 bp, Homo sapiens
  Putative CMYB/CMYB.01 motif
tttcacaacagttggagg

SEQ ID NO: 180 - DNA, 9 bp, Homo sapiens
  Putative VMYB/VMYB.02 motif
tccaactgt

SEQ ID NO: 181 - DNA, 14 bp, Homo sapiens
  Putative CEBP/CEBPB.01 motif
ctgttgtgaaagcc

SEQ ID NO: 182 - DNA, 21 bp, Homo sapiens
  Putative MINI/MUSCLE_INI.02 motif
cctccaccccacccagctctg

SEQ ID NO: 183 - DNA, 11 bp, Homo sapiens
  Putative EBOX/SREBP1.02 motif
ctccaccccac

SEQ ID NO: 184 - DNA, 24 bp, Homo sapiens
  Putative PAX5/PAX9.01 motif
aagagccagagctgggtggggtgg

SEQ ID NO: 185 - DNA, 14 bp, Homo sapiens
  Putative SP1F/GC.01 motif
gctgggtggggtgg

SEQ ID NO: 186 - DNA, 10 bp, Homo sapiens
  Putative NFKB/CREL.01 motif
tggctcttcc

SEQ ID NO: 187 - DNA, 12 bp, Homo sapiens
  Putative ETSF/GABP.01 motif
ggaggaagagcc

SEQ ID NO: 188 - DNA, 19 bp, Homo sapiens
  Putative SEF1/SEF1.01 motif
ctccaggacatctggggta

SEQ ID NO: 189 - DNA, 16 bp, Homo sapiens
  Putative AP4R/TALIALPHAE47.01 motif
taccccagatgtcctg

SEQ ID NO: 190 - DNA, 18 bp, Homo sapiens
  Putative REOA/POLYA.01 motif
caatacatccatgatcta

SEQ ID NO: 191 - DNA, 11 bp, Homo sapiens
  Putative EVI1/EVI1.02 motif
agacaagaaga

SEQ ID NO: 192 - DNA, 18 bp, Homo sapiens
  Putative CMYB/CMYB.01 motif
tctaagagctgttgccag

SEQ ID NO: 193 - DNA, 17 bp, Homo sapiens
  Putative XBBF/RFX1.01 motif
tggactcctggcaacag

SEQ ID NO: 194 - DNA, 18 bp, Homo sapiens
  Putative MYOF/NF1.01 motif
cgttggctggactcctgg

SEQ ID NO: 195 - DNA, 12 bp, Homo sapiens
  Putative EGRF/EGR3.01 motif
gagcgttggctg

SEQ ID NO: 196 - DNA, 22 bp, Homo sapiens
  Putative NOLF/OLF1.01 motif
aacgagtccctttgggcttcct

SEQ ID NO: 197 - DNA, 9 bp, Homo sapiens
  Putative AREB/AREB6.04 motif
ctgtttgga

SEQ ID NO: 198 - DNA, 27 bp, Homo sapiens
  Putative GREF/ARE.01 motif
gtttgatgttccttgtgttccctttcc

SEQ ID NO: 199 - DNA, 13 bp, Homo sapiens
  Putative IRFF/IRF2.01 motif
ggaaagggaacac

SEQ ID NO: 200 - DNA, 16 bp, Homo sapiens
  Putative LDPS/LDSPOLYA.01 motif
tccttgtgttcccttt

SEQ ID NO: 201 - DNA, 18 bp, Homo sapiens
  Putative XBBF/RFX1.02 motif
agggaacacaaggaacat

SEQ ID NO: 202 - DNA, 10 bp, Homo sapiens
  Putative RPOA/DTYPEPA.01 motif
aacatcaaac

SEQ ID NO: 203 - DNA, 13 bp, Homo sapiens
  Putative IKRS/IK1.01 motif
gtgtgggaaggtt

SEQ ID NO: 204 - DNA, 21 bp, Homo sapiens
  Putative XSEC/STAF.02 motif
ccttcccacactgctctacat

SEQ ID NO: 205 - DNA, 10 bp, Homo sapiens
  Putative RPOA/DTYPEPA.01 motif
accacaaaac

SEQ ID NO: 206 - DNA, 6 bp, Homo sapiens
  Putative HAML/AML1.01 motif
tgtggt

SEQ ID NO: 207 - DNA, 6 bp, Homo sapiens
  Putative HAML/AML1.01 motif
tgtggt

SEQ ID NO: 208 - DNA, 14 bp, Homo sapiens
  Putative ECAT/NFY.03 motif
atcaacaaatcagc

SEQ ID NO: 209 - DNA, 10 bp, Homo sapiens
  Putative TBPF/ATATA.01 motif
ttatttcagt

SEQ ID NO: 210 - DNA, 13 bp, Homo sapiens
  Putative IRFF/IRF1.01 motif
aaaaactgaaata

SEQ ID NO: 211 - DNA, 10 bp, Homo sapiens
  Putative VMYB/VMYB.01 motif
aaaaactgaa

SEQ ID NO: 212 - DNA, 21 bp, Homo sapiens
  Putative PAX6/PAX6.01 motif
agttttttcgctgcatttaga

SEQ ID NO: 213 - DNA, 8 bp, Homo sapiens
  Putative E2FF/E2F.02 motif
gcgaaaaa

SEQ ID NO: 214 - DNA, 23 bp, Homo sapiens
  Putative PAX5/PAX9.01 motif
tctacccatggaagtgtcaggaa

SEQ ID NO: 215 - DNA, 15 bp, Homo sapiens
  Putative MTF1/MTF-1.01 motif
tcctgcacacttcca

SEQ ID NO: 216 - DNA, 14 bp, Homo sapiens
  Putative ETSF/ETS2.01 motif
tgcaggaagatgga

SEQ ID NO: 217 - DNA, 13 bp, Homo sapiens
  Putative ZFIA/ZID.01 motif
tgactccatcttc

SEQ ID NO: 218 - DNA, 11 bp, Homo sapiens
  Putative AP1F/AP1FJ.01 motif
ggtgactccat

SEQ ID NO: 219 - DNA, 9 bp, Homo sapiens
  Putative VMYB/VMYB.02 motif
ccaaacggg

SEQ ID NO: 220 - DNA, 16 bp, Homo sapiens
  Putative ETSF/ELK1.01 motif
caaacgggatgatcca

SEQ ID NO: 221 - DNA, 12 bp, Homo sapiens
  Putative NFKB/NFKAPPAB.02 motif
cgggatgatcca

SEQ ID NO: 222 - DNA, 9 bp, Homo sapiens
  Putative AREB/AREB6.04 motif
ctgtttctt

SEQ ID NO: 223 - DNA, 13 bp, Homo sapiens
  Putative ZFI1A/ZID.01 motif
cggctctaacaca

SEQ ID NO: 224 - DNA, 18 bp, Homo sapiens
  Putative XBBF/RFX1.02 motif
ctctaacacaagcaacag

SEQ ID NO: 225 - DNA, 18 bp, Homo sapiens
  Putative CMYB/CMYB.01 motif
gtttgttgctgttgcttg

SEQ ID NO: 226 - DNA, 15 bp, Homo sapiens
  Putative CREB/TAXCREB.02 motif
gaggaaatacgtctt

SEQ ID NO: 227 - DNA, 14 bp, Homo sapiens
  Putative ETSF/ETS2.01 motif
aagaggaaatacgt

SEQ ID NO: 228 - DNA, 12 bp, Homo sapiens
  Putative NFAT/NFAT.01 motif
aagaggaaatac

SEQ ID NO: 229 - DNA, 11 bp, Homo sapiens
  Putative EVI1/EVI1.02 motif
tgagaagatta

SEQ ID NO: 230 - DNA, 16 bp, Homo sapiens
  Putative OAZF/ROAZ.01 motif
cagcatccttaggtga

SEQ ID NO: 231 - DNA, 11 bp, Homo sapiens
  Putative EBOR/DELTAEF1.01 motif
cctcacctaag

SEQ ID NO: 232 - DNA, 8 bp, Homo sapiens
  Putative CREB/CREBP1.01 motif
tcacctaa

SEQ ID NO: 233 - DNA, 15 bp, Homo sapiens
  Putative HNF4/HNF4.02 motif
tgggtccagaggcct

SEQ ID NO: 234 - DNA, 11 bp, Homo sapiens
  Putative GATA/GATA.01 motif
agataaggcct

SEQ ID NO: 235 - DNA, 12 bp, Homo sapiens
  Putative CREB/E4BP4.01 motif
ccttatctaaaa

SEQ ID NO: 236 - DNA, 10 bp, Homo sapiens
  Putative TBPF/ATATA.01 motif
ttagataagg

SEQ ID NO: 237 - DNA, 18 bp, Homo sapiens
  Putative XBBF/MIF1.01 motif
acggtgcccagccaccca

SEQ ID NO: 238 - DNA, 8 bp, Homo sapiens
  Putative EBOX/USF.02 motif
acacatgt

SEQ ID NO: 239 - DNA, 10 bp, Homo sapiens
  Putative VBPF/VBP.01 motif
attacatgtg

SEQ ID NO: 240 - DNA, 12 bp, Homo sapiens
  Putative IKRS/IK2.01 motif
tgctgggattac

SEQ ID NO: 241 - DNA, 21 bp, Homo sapiens
  Putative NRSF/NRSF.01 motif
cccagcactttggaaggccga

SEQ ID NO: 242 - DNA, 19 bp, Homo sapiens
  Putative TANT/TANTIGEN.01 motif
ggaaggccgaggcaggtgg

SEQ ID NO: 243 - DNA, 13 bp, Homo sapiens
  Putative AREB/AREB6.01 motif
gatccacctgcct

SEQ ID NO: 244 - DNA, 10 bp, Homo sapiens
  Putative MYOD/MYOD.02 motif
tccacctgcc

SEQ ID NO: 245 - DNA, 11 bp, Homo sapiens
  Putative EBOX/SREBP1.02 motif
gatcacccgag

SEQ ID NO: 246 - DNA, 10 bp, Homo sapiens
  Putative RARF/RAR.01 motif
aggtcaggag

SEQ ID NO: 247 - DNA, 10 bp, Homo sapiens
  Putative CREB/HLF.01 motif
gtttcgccat

SEQ ID NO: 248 - DNA, 10 bp, Homo sapiens
  Putative CLOX/CDPCR3HD.01 motif
tattgatgag

SEQ ID NO: 249 - DNA, 10 bp, Homo sapiens
  Putative OCT1/OCT1.02 motif
aatgcaaaaa

SEQ ID NO: 250 - DNA, 12 bp, Homo sapiens
  Putative MYT1/MYT1.01 motif
aaaaattagctt

SEQ ID NO: 251 - DNA, 6 bp, Homo sapiens
  Putative HAML/AML1.01 motif
tgtggt

SEQ ID NO: 252 - DNA, 12 bp, Homo sapiens
  Putative IKRS/IK2.01 motif
ggctgggattac

SEQ ID NO: 253 - DNA, 19 bp, Homo sapiens
  Putative AHRR/AHRARNT.02 motif
tgggtttgagtgattctcc

SEQ ID NO: 254 - DNA, 13 bp, Homo sapiens
  Putative CHOP/CHOP.01 motif
cactgcaatctcc

SEQ ID NO: 255 - DNA, 19 bp, Homo sapiens
  Putative  OCT1/OCT1.01motif
gagattatgccactgcact

SEQ ID NO: 256 - DNA, 16 bp, Homo sapiens
  Putative MEF2/MEF2.01 motif
ctcaaaaaataaaata

SEQ ID NO: 257 - DNA, 19 bp, Homo sapiens
  Putative CDXF/CDX2.01 motif
caaaggttttattttattt

SEQ ID NO: 258 - DNA, 11 bp, Homo sapiens
  Putative EVI1/EVI1.03 motif
aaataaaataa

SEQ ID NO: 259 - DNA, 18 bp, Homo sapiens
  Putative RPOA/POLYA.01 motif
aaataaaacctttggggc

SEQ ID NO: 260 - DNA, 8 bp, Homo sapiens
  Putative E2FF/E2F.02 motif
gccccaaa

SEQ ID NO: 261 - DNA, 22 bp, Homo sapiens
  Putative XSEC/STAF.01 motif
aatccccagaattctggactct

SEQ ID NO: 262 - DNA, 12 bp, Homo sapiens
  Putative NFKB/NFKAPPAB.02 motif
ggggattttcaa

SEQ ID NO: 263 - DNA, 17 bp, Homo sapiens
  Putative HNF1/HNF1.02 motif
ggctattcaataaatgg

SEQ ID NO: 264 - DNA, 8 bp, Homo sapiens
  Putative RPOA/LPOLYA.01 motif
caataaat

SEQ ID NO: 265 - DNA, 15 bp, Homo sapiens
  Putative TBPF/TATA.01 motif
atataaatcccattt

SEQ ID NO: 266 - DNA, 9 bp, Homo sapiens
  Putative HMTB/MTBF.01 motif
tgggattta

SEQ ID NO: 267 - DNA, 10 bp, Homo sapiens
  Putative CREB/HLF.01 motif
gttatgtgat

SEQ ID NO: 268 - DNA, 10 bp, Homo sapiens
  Putative VBPF/VBP.01 motif
gttatgtgat

SEQ ID NO: 269 - DNA, 12 bp, Homo sapiens
  Putative CREB/CREB.03 motif
tctgacgcagtt

SEQ ID NO: 270 - DNA, 14 bp, Homo sapiens
  Putative GATA/GATA1.01 motif
tagttgataggaga

SEQ ID NO: 271 - DNA, 15 bp, Homo sapiens
  Putative CLOX/CLOX.01 motif
aaaatcgaatagttg

SEQ ID NO: 272 - DNA, 12 bp, Homo sapiens
  Putative NFAT/NFAT.01 motif
tgaaggaaaatc

SEQ ID NO: 273 - DNA, 24 bp, Homo sapiens
  Putative GFI1/GFI1.01 motif
aatttaaaaatcacatcaagggat

SEQ ID NO: 274 - DNA, 10 bp, Homo sapiens
  Putative MEF2/MEF2.05 motif
aatttaaaaa

SEQ ID NO: 275 - DNA, 10 bp, Homo sapiens
  Putative GATA/GATA3.02 motif
agggatctaa

SEQ ID NO: 276 - DNA, 16 bp, Homo sapiens
  Putative FKHD/FREAC3.01 motif
gggatctaaataaaga

SEQ ID NO: 277 - DNA, 10 bp, Homo sapiens
  Putative MEF2/MEF2.05 motif
gatctaaata

SEQ ID NO: 278 - DNA, 8 bp, Homo sapiens
  Putative RPOA/LPOLYA.01 motif
aaataaag

SEQ ID NO: 279 - DNA, 9 bp, Homo sapiens
  Putative HMTB/MTBF.01 motif
agctattta

SEQ ID NO: 280 - DNA, 9 bp, Homo sapiens
  Putative VMYB/VMYB.02 motif
cccaactga

SEQ ID NO: 281 - DNA, 8 bp, Homo sapiens
  Putative SMAD/SMAD3.01 motif
gtctggtc

SEQ ID NO: 282 - DNA, 15 bp, Homo sapiens
  Putative HNF4/HNF4.02 motif
aaggaccaaacctct

SEQ ID NO: 283 - DNA, 11 bp, Homo sapiens
  Putative MYT1/MYT1.02 motif
agaaagttcta

SEQ ID NO: 284 - DNA, 10 bp, Homo sapiens
  Putative HEAT/HSF1.01 motif
agaaagttct

SEQ ID NO: 285 - DNA, 8 bp, Homo sapiens
  Putative MZF1/MZF1.01 motif
aatgggga

SEQ ID NO: 286 - DNA, 10 bp, Homo sapiens
  Putative TBPF/TATA.02 motif
tctgtaaaat

SEQ ID NO: 287 - DNA, 13 bp, Homo sapiens
  Putative GATA/GATA1.03 motif
tacagataaaggg

SEQ ID NO: 288 - DNA, 16 bp, Homo sapiens
  Putative ETSF/PU1.01 motif
gaatgaggaagggtaa

SEQ ID NO: 289 - DNA, 10 bp, Homo sapiens
  Putative CREB/HLF.01 motif
gttacttcat

SEQ ID NO: 290 - DNA, 10 bp, Homo sapiens
  Putative VBPF/VBP.01 motif
gttacttcat

SEQ ID NO: 291 - DNA, 13 bp, Homo sapiens
  Putative RORA/RORA2.01 motif
gtaacttggtcaa

SEQ ID NO: 292 - DNA, 16 bp, Homo sapiens
  Putative LDPS/LDSPOLYA.01 motif
ggagtgtgtgtgcatg

SEQ ID NO: 293 - DNA, 8 bp, Homo sapiens
  Putative EBOX/USF.02 motif
acacatgc

SEQ ID NO: 294 - DNA, 10 bp, Homo sapiens
  Putative NFKB/NFKAPPAB.01 motif
gggggtgccc

SEQ ID NO: 295 - DNA, 21 bp, Homo sapiens
  Putative MINI/MUSCLE_INI.03 motif
ggcaccccccaccccgacccc

SEQ ID NO: 296 - DNA, 21 bp, Homo sapiens
  Putative REBV/EBVR.01 motif
ggggtcggggtggggggtgcc

SEQ ID NO: 297 - DNA, 13 bp, Homo sapiens
  Putative EGRF/WT1.01 motif
gggtggggggtgc

SEQ ID NO: 298 - DNA, 14 bp, Homo sapiens
  Putative SP1F/GC.01 motif
tcggggtggggggt

SEQ ID NO: 299 - DNA, 14 bp, Homo sapiens
  Putative RREB/RREB1.01 motif
ccccaccccgaccc

SEQ ID NO: 300 - DNA, 9 bp, Homo sapiens
  Putative PCAT/ACAAT.01 motif
ccaccactg

SEQ ID NO: 301 - DNA, 16 bp, Homo sapiens
  Putative ARP1/ARP1.01 motif
tgattccttgctctca

SEQ ID NO: 302 - DNA, 11 bp, Homo sapiens
  Putative MYT1/MYT1.02 motif
tcaaagttgtt

SEQ ID NO: 303 - DNA, 15 bp, Homo sapiens
  Putative IRFF/ISRE.01 motif
ctgtaccagaaactc

SEQ ID NO: 304 - DNA, 13 bp, Homo sapiens
  Putative EGRF/WT1.01 motif
gtgtgggaggctc

SEQ ID NO: 305 - DNA, 10 bp, Homo sapiens
  Putative RARF/RAR.01 motif
aggtcaccca

SEQ ID NO: 306 - DNA, 13 bp, Homo sapiens
  Putative RORA/RORA1.01 motif
agaagaaggtcac

SEQ ID NO: 307 - DNA, 16 bp, Homo sapiens
  Putative EVI1/EVI1.01 motif
agccaagagaagaagg

SEQ ID NO: 308 - DNA, 14 bp, Homo sapiens
  Putative OCT1/OCT1.05 motif
ctcattttaattca

SEQ ID NO: 309 - DNA, 15 bp, Homo sapiens
  Putative OCTB/TST1.01 motif
agtgaattaaaatga

SEQ ID NO: 310 - DNA, 13 bp, Homo sapiens
  Putative RBIT/BRIGHT.01 motif
agtgaattaaaat

SEQ ID NO: 311 - DNA, 8 bp, Homo sapiens
  Putative NKXH/NKX25.02 motif
tttaattc

SEQ ID NO: 312 - DNA, 27 bp, Homo sapiens
  Putative GREF/PRE.01 motif
ttcatagtgttgttttgttctcgtttt

SEQ ID NO: 313 - DNA, 18 bp, Homo sapiens
  Putative RPOA/POLYA.01 motif
gaacaaaacaacactatg

SEQ ID NO: 314 - DNA, 18 bp, Homo sapiens
  Putative AHRR/AHR.01 motif
actccagcttgggtgaga

SEQ ID NO: 315 - DNA, 24 bp, Homo sapiens
  Putative GFI1/GFI1.01 motif
agtgctgcaatcacagctcattgc

SEQ ID NO: 316 - DNA, 9 bp, Homo sapiens
  Putative LYMF/LYF1.01 motif
tttgggagg

SEQ ID NO: 317 - DNA, 32 bp, Homo sapiens
  Putative HOBO/HOGNESS.01 motif
cacggtggctcacacctgtaatcccagcactt

SEQ ID NO: 318 - DNA, 12 bp, Homo sapiens
  Putative IKRS/IK2.01 motif
tgctgggattac

SEQ ID NO: 319 - DNA, 16 bp, Homo sapiens
  Putative MYOD/E47.02 motif
gattacaggtgtgagc

SEQ ID NO: 320 - DNA, 12 bp, Homo sapiens
  Putative AREB/AREB6.02 motif
tcacacctgtaa

SEQ ID NO: 321 - DNA, 12 bp, Homo sapiens
  Putative BRAC/TBX5.01 motif
acaggtgtgagc

SEQ ID NO: 322 - DNA, 17 bp, Homo sapiens
  Putative TBPF/MTATA.01 motif
ctgtttaaaaccctata

SEQ ID NO: 323 - DNA, 16 bp, Homo sapiens
  Putative FKHD/FREAC2.01 motif
gggttttaaacagtaa

SEQ ID NO: 324 - DNA, 10 bp, Homo sapiens
  Putative MEF2/MEF2.05 motif
gttttaaaca

SEQ ID NO: 325 - DNA, 18 bp, Homo sapiens
  Putative CEBP/CEBP.02 motif
tgcctgcggtaagtcgta

SEQ ID NO: 326 - DNA, 22 bp, Homo sapiens
  Putative NOLF/OLF1.01 motif
aaagggtccccccggggcctgt

SEQ ID NO: 327 - DNA, 12 bp, Homo sapiens
  Putative AP2F/AP2.01 motif
gtccccccgggg

SEQ ID NO: 328 - DNA, 8 bp, Homo sapiens
  Putative MZF1/MZF1.01 motif
cgggggga

SEQ ID NO: 329 - DNA, 22 bp, Homo sapiens
  Putative HEN1/HEN1.01 motif
ccagggtacagctgtgacaccg

SEQ ID NO: 330 - DNA, 10 bp, Homo sapiens
  Putative AP4R/AP4.01 motif
cacagctgta

SEQ ID NO: 331 - DNA, 14 bp, Homo sapiens
  Putative GATA/GATA1.02 motif
actgggataatcca

SEQ ID NO: 332 - DNA, 12 bp, Homo sapiens
  Putative NFKB/NFKAPPAB.02 motif
tgggataatcca

SEQ ID NO: 333 - DNA, 13 bp, Homo sapiens
  Putative FKHD/HFH8.01 motif
tagataaacaaaa

SEQ ID NO: 334 - DNA, 11 bp, Homo sapiens
  Putative GATA/GATA.01 motif
agtaaaacaaa

SEQ ID NO: 335 - DNA, 12 bp, Homo sapiens
  Putative SORY/SRY.01 motif
ataaacaaaaat

SEQ ID NO: 336 - DNA, 12 bp, Homo sapiens
  Putative CREB/CREB.02 motif
ggaatgacgatc

SEQ ID NO: 337 - DNA, 13 bp, Homo sapiens
  Putative PAX3/PAX3.01 motif
tcgtcattccatt

SEQ ID NO: 338 - DNA, 12 bp, Homo sapiens
  Putative TEAF/TEF1.01 motif
gtcattccattt

SEQ ID NO: 339 - DNA, 18 bp, Homo sapiens
  Putative PAX1/PAX1.01 motif
ccatttctctctgtatat

SEQ ID NO: 340 - DNA, 12 bp, Homo sapiens
  Putative NFAT/NFAT.01 motif
gcttggaaaaat

SEQ ID NO: 341 - DNA, 15 bp, Homo sapiens
  Putative BARB/BARBIE.01 motif
atgaaaagggcttgg

SEQ ID NO: 342 - DNA, 10 bp, Homo sapiens
  Putative OCT1/OCT1.02 motif
catgaaaagg

SEQ ID NO: 343 - DNA, 22 bp, Homo sapiens
  Putative AP1F/TCF11MAFG.01 motif
ttttcatgaatgatcagttatt

SEQ ID NO: 344 - DNA, 10 bp, Homo sapiens
  Putative PITI1/PIT1.01 motif
gatcattcat

SEQ ID NO: 345 - DNA, 10 bp, Homo sapiens
  Putative VMYB/VMYB.01 motif
aataactgat

SEQ ID NO: 346 - DNA, 14 bp, Homo sapiens
  Putative ETSF/ETS2.01 motif
tgcaggaaataact

SEQ ID NO: 347 - DNA, 24 bp, Homo sapiens
  Putative GFI1/GFI1.01 motif
aaaaaaaaaatcagtgcaggaaat

SEQ ID NO: 348 - DNA, 11 bp, Homo sapiens
  Putative AP1F/AP1FJ.01 motif
ggtgacagagt

SEQ ID NO: 349 - DNA, 11 bp, Homo sapiens
  Putative EBOX/SREBP1.02 motif
gatcatgccac

SEQ ID NO: 350 - DNA, 13 bp, Homo sapiens
  Putative PAX3/PAX3.01 motif
tcggctcgctgca

SEQ ID NO: 351 - DNA, 10 bp, Homo sapiens
  Putative HEAT/HSF1.01 motif
agaagaatcg

SEQ ID NO: 352 - DNA, 21 bp, Homo sapiens
  Putative XSEC/STAF.02 motif
gagtaccatcatgcccggcta

SEQ ID NO: 353 - DNA, 20 bp, Homo sapiens
  Putative P53F/P53.01 motif
catcatgcccggctaatttt

SEQ ID NO: 354 - DNA, 17 bp, Homo sapiens
  Putative MEF2/RSRFC4.02 motif
ctactaaaaatacaaaa

SEQ ID NO: 355 - DNA, 18 bp, Homo sapiens
  Putative SRFF/SRF.01 motif
ttcaccatattggccagg

SEQ ID NO: 356 - DNA, 11 bp, Homo sapiens
  Putative ECAT/NFY.02 motif
tggccaatatg

SEQ ID NO: 357 - DNA, 15 bp, Homo sapiens
  Putative HNF4/HNF4.02 motif
cagatcgcaaggtcc

SEQ ID NO: 358 - DNA, 9 bp, Homo sapiens
  Putative LYMF/LYF1.01 motif
tttgggagg

SEQ ID NO: 359 - DNA, 32 bp, Homo sapiens
  Putative HOBO/HOGNESS.01 motif
cgcggtggctcacgcctgtaatcccagcactt

SEQ ID NO: 360 - DNA, 12 bp, Homo sapiens
  Putative IKRS/IK2.01 motif
tgctgggattac

SEQ ID NO: 361 - DNA, 15 bp, Homo sapiens
  Putative CREB/TAXCREB.01 motif
ggtggctcacgcctg

SEQ ID NO: 362 - DNA, 10 bp, Homo sapiens
  Putative EBOX/MYCMAX.03 motif
gccaggcgcg

SEQ ID NO: 363 - DNA, 10 bp, Homo sapiens
  Putative GATA/GATA3.02 motif
actgatataa

SEQ ID NO: 364 - DNA, 15 bp, Homo sapiens
  Putative EVI1/EVI1.04 motif
tgatataaaaagaat

SEQ ID NO: 365 - DNA, 10 bp, Homo sapiens
  Putative MEF2/MEF2.05 motif
gatataaaaa

SEQ ID NO: 366 - DNA, 15 bp, Homo sapiens
  Putative TBPF/TATA.01 motif
atataaaaagaattt

SEQ ID NO: 367 - DNA, 15 bp, Homo sapiens
  Putative RPOA/APOLYA.01 motif
aaaaaaattcttttt

SEQ ID NO: 368 - DNA, 10 bp, Homo sapiens
  Putative MEF2/MEF2.05 motif
aatttaaaaa

SEQ ID NO: 369 - DNA, 11 bp, Homo sapiens
  Putative EBOX/SREBP1.02 motif
tttctccccac

SEQ ID NO: 370 - DNA, 8 bp, Homo sapiens
  Putative MZF1/MZF1.01 motif
agtgggga

SEQ ID NO: 371 - DNA, 21 bp, Homo sapiens
  Putative MINI/MUSCLE_INI.03 motif
ccccactcccacccccaggct

SEQ ID NO: 372 - DNA, 14 bp, Homo sapiens
  Putative RREB/RREB1.01 motif
ccccactcccaccc

SEQ ID NO: 373 - DNA, 13 bp, Homo sapiens
  Putative EGRF/WT1.01 motif
gggtgggagtggg

SEQ ID NO: 374 - DNA, 12 bp, Homo sapiens
  Putative AP2F/AP2.01 motif
cacccccaggct

SEQ ID NO: 375 - DNA, 17 bp, Homo sapiens
  Putative TBPF/MTATA.01 motif
ccttataaagcagcctc

SEQ ID NO: 376 - DNA, 6 bp, Homo sapiens
  Putative HAML/AMLI.01 motif
tgtggt

SEQ ID NO: 377 - DNA, 14 bp, Homo sapiens
  Putative ETSF/ELK1.02 motif
gggcccggaattgg

SEQ ID NO: 378 - DNA, 16 bp, Homo sapiens
  Putative LYMF/THIE47.01 motif
aattgggtctggggca

SEQ ID NO: 379 - DNA, 28 bp, Homo sapiens
  Putative PAX5/PAX5.01 motif
cccaagagcagggcagagaagcaagcaa

SEQ ID NO: 380 - DNA, 23 bp, Homo sapiens
  Putative LTUP/TAACC.01 motif
tgcccctgaggctaaccccaaga

SEQ ID NO: 381 - DNA, 28 bp, Homo sapiens
  Putative PAX5/PAX5.01 motif
ctcaggggcagggttgagagtcaggctt

SEQ ID NO: 382 - DNA, 25 bp, Homo sapiens
  Putative PCAT/CLTR_CAAT.01 motif
gccaagcctgactctcaaccctgcc

SEQ ID NO: 383 - DNA, 12 bp, Homo sapiens
  Putative MYOD/MYF5.01 motif
aggcagcaggag

SEQ ID NO: 384 - DNA, 16 bp, Homo sapiens
  Putative ETSF/ELK1.01 motif
gcagcaggaggtccag

SEQ ID NO: 385 - DNA, 8 bp, Homo sapiens
  Putative SMAD/SMAD3.01 motif
gtctggac

SEQ ID NO: 386 - DNA, 10 bp, Homo sapiens
  Putative GATA/GATA2.02 motif
ggagatacca

SEQ ID NO: 387 - DNA, 9 bp, Homo sapiens
  Putative HMTB/MTBF.01 motif
tggtatctc

SEQ ID NO: 388 - DNA, 13 bp, Homo sapiens
  Putative EGRF/WT1.01 motif
gagagggcgcatc

SEQ ID NO: 389 - DNA, 20 bp, Homo sapiens
  Putative PERO/PPARA.01 motif
ctgaaacaggaaaaaggcag

SEQ ID NO: 390 - DNA, 14 bp, Homo sapiens
  Putative GKLF/GKLF.01 motif
aaacaggaaaaagg

SEQ ID NO: 391 - DNA, 12 bp, Homo sapiens
  Putative NFAT/NFAT.01 motif
aacaggaaaaag

SEQ ID NO: 392 - DNA, 9 bp, Homo sapiens
  Putative AREB/AREB6.04 motif
ctgtttcag

SEQ ID NO: 393 - DNA, 12 bp, Homo sapiens
  Putative SORY/SRY.01 motif
aaaaacaaaaca

SEQ ID NO: 394 - DNA, 12 bp, Homo sapiens
  Putative FKHD/HFH2.01 motif
aaaaaaacaaaa

SEQ ID NO: 395 - DNA, 13 bp, Homo sapiens
  Putative EGRF/WT1.01 motif
gagagggagggag

SEQ ID NO: 396 - DNA, 13 bp, Homo sapiens
  Putative EGRF/WT1.01 motif
gagagggagggag

SEQ ID NO: 397 - DNA, 14 bp, Homo sapiens
  Putative GKLF/GKLF.01 motif
agagagagagaggg

SEQ ID NO: 398 - DNA, 13 bp, Homo sapiens
  Putative SP1F/SP1.01 motif
ggagggaggggga

SEQ ID NO: 399 - DNA, 14 bp, Homo sapiens
  Putative GKLF/GKLF.01 motif
gaaggagggagggg

SEQ ID NO: 400 - DNA, 10 bp, Homo sapiens
  Putative OCT1/OCT1.02 motif
gatgcacata

SEQ ID NO: 401 - DNA, 9 bp, Homo sapiens
  Putative EVI1/EVI1.06 motif
acaaggtag

SEQ ID NO: 402 - DNA, 13 bp, Homo sapiens
  Putative TCFF/TCF11.01 motif
gtcatcctgctgt

SEQ ID NO: 403 - DNA, 21 bp, Homo sapiens
  Putative MINI/MUSCLE_INI.01 motif
tccctcctccacaccagcaga

SEQ ID NO: 404 - DNA, 21 bp, Homo sapiens
  Putative NRSF/NRSF.01 motif
ttcagcaacaagaatagccga

SEQ ID NO: 405 - DNA, 15 bp, Homo sapiens
  Putative CLOX/CDPCR3.01 motif
cagcaacaagaatag

SEQ ID NO: 406 - DNA, 25 bp, Homo sapiens
  Putative PCAT/CLTR_CAAT.01 motif
cccaagaagcatcctgcaggctttc

SEQ ID NO: 407 - DNA, 15 bp, Homo sapiens
  Putative BARB/BARBIE.01 motif
tcaaaaagcagaaag

SEQ ID NO: 408 - DNA, 16 bp, Homo sapiens
  Putative MEF2/MMEF2.01 motif
tgctttaaaatacact

SEQ ID NO: 409 - DNA, 10 bp, Homo sapiens
  Putative TBPF/TATA.02 motif
gctttaaaat

SEQ ID NO: 410 - DNA, 10 bp, Homo sapiens
  Putative TBPF/ATATA.01 motif
ctatgtatgc

SEQ ID NO: 411 - DNA, 12 bp, Homo sapiens
  Putative MYT1/MYT1.01 motif
catagttaactg

SEQ ID NO: 412 - DNA, 10 bp, Homo sapiens
  Putative GATA/GATA3.02 motif
ctagatgtta

SEQ ID NO: 413 - DNA, 14 bp, Homo sapiens
  Putative FKHD/XFD3.01 motif
aaggttaacatcta

SEQ ID NO: 414 - DNA, 12 bp, Homo sapiens
  Putative MYT1/MYT1.01 motif
aaaggttaacat

SEQ ID NO: 415 - DNA, 16 bp, Homo sapiens
  Putative AP4R/TAL1BETA-E47.01 motif
aaacacagatggaggc

SEQ ID NO: 416 - DNA, 12 bp, Homo sapiens
  Putative EGRF/EGR1.01 motif
ttctgtgggcgg

SEQ ID NO: 417 - DNA, 13 bp, Homo sapiens
  Putative ZFIA/ZID.01 motif
cggctccagcctc

SEQ ID NO: 418 - DNA, 15 bp, Homo sapiens
  Putative CREB/TAXCREB.02 motif
cgggatctgcgggaa

SEQ ID NO: 419 - DNA, 18 bp, Homo sapiens
  Putative CEBP/CEBP.02 motif
gatctgcgggaagacacg

SEQ ID NO: 420 - DNA, 15 bp, Homo sapiens
  Putative E2FF/E2F.01 motif
tctgcgggaagacac

SEQ ID NO: 421 - DNA, 12 bp, Homo sapiens
  Putative EBOX/NMYC.01 motif
ttccccgtgtct

SEQ ID NO: 422 - DNA, 12 bp, Homo sapiens
  Putative CLOX/CDP.01 motif
tcattaatcaaa

SEQ ID NO: 423 - DNA, 15 bp, Homo sapiens
  Putative HNF1/HNF1.01 motif
gattaatgatttatt

SEQ ID NO: 424 - DNA, 18 bp, Homo sapiens
  Putative CART/CART1.01 motif
gatttattttgattaacg

SEQ ID NO: 425 - DNA, 8 bp, Homo sapiens
  Putative RPOA/LPOLYA.01 motif
aaataaat

SEQ ID NO: 426 - DNA, 15 bp, Homo sapiens
  Putative HNF1/HNF1.01 motif
cgttaatcaaaataa

SEQ ID NO: 427 - DNA, 24 bp, Homo sapiens
  Putative COMP/COMP1.01 motif
tattttgattaacgccgtcacagt

SEQ ID NO: 428 - DNA, 14 bp, Homo sapiens
  Putative CREB/ATF.01 motif
ctgtgacggcgtta

SEQ ID NO: 429 - DNA, 28 bp, Homo sapiens
  Putative PAX5/PAX5.02 motif
agggactgctctaaggcgtcactgtgac

SEQ ID NO: 430 - DNA, 21 bp, Homo sapiens
  Putative PAX6/PAX6.01 motif
cacagtgacgccttagagcag

SEQ ID NO: 431 - DNA, 14 bp, Homo sapiens
  Putative CREB/ATF.01 motif
cagtgacgccttag

SEQ ID NO: 432 - DNA, 11 bp, Homo sapiens
  Putative WHZF/WHN.01 motif
agtgacgcctt

SEQ ID NO: 433 - DNA, 16 bp, Homo sapiens
  Putative FKHD/FREAC4.01 motif
cccgggtgaacaggga

SEQ ID NO: 434 - DNA, 12 bp, Homo sapiens
  Putative EGRF/NGFIC.01 motif
cagcgagggtgg

SEQ ID NO: 435 - DNA, 13 bp, Homo sapiens
  Putative SP1F/SP1.01 motif
tgggggcggacgc

SEQ ID NO: 436 - DNA, 14 bp, Homo sapiens
  Putative GKLF/GKLF.01 motif
ggaaagaggagggg

SEQ ID NO: 437 - DNA, 25 bp, Homo sapiens
  Putative PCAT/CLTR_CAAT.01 motif
accaaggccccgcccctcctctttc

SEQ ID NO: 438 - DNA, 13 bp, Homo sapiens
  Putative SP1F/SP1.01 motif
gaggggcggggcc

SEQ ID NO: 439 - DNA, 14 bp, Homo sapiens
  Putative RREB/RREB1.01 motif
ccccacccgaccaa

SEQ ID NO: 440 - DNA, 12 bp, Homo sapiens
  Putative TEAF/TEF1.01 motif
cccattccatac

SEQ ID NO: 441 - DNA, 24 bp, Homo sapiens
  Putative PAX5/PAX9.01 motif
aatgggcagggtgggggggatggg

SEQ ID NO: 442 - DNA, 14 bp, Homo sapiens
  Putative RREB/RREB1.01 motif
ccccaccctgccca

SEQ ID NO: 443 - DNA, 13 bp, Homo sapiens
  Putative EGRF/WT1.01 motif
gggtgggggggat

SEQ ID NO: 444 - DNA, 14 bp, Homo sapiens
  Putative RREB/RREB1.01 motif
gcccatccccccca

SEQ ID NO: 445 - DNA, 8 bp, Homo sapiens
  Putative MZF1/MZF1.01 motif
ggggggga

SEQ ID NO: 446 - DNA, 13 bp, Homo sapiens
  Putative SP1F/SP1.01 motif
gatgggcggggta

SEQ ID NO: 447 - DNA, 13 bp, Homo sapiens
  Putative SP1F/SP1.01 motif
gatgggcggggcc

SEQ ID NO: 448 - DNA, 13 bp, Homo sapiens
  Putative E2FF/E2F.03 motif
gcccgggaaattc

SEQ ID NO: 449 - DNA, 22 bp, Homo sapiens
  Putative NOLF/OLF1.01 motif
ggaaattccccggcgcgggcag

SEQ ID NO: 450 - DNA, 10 bp, Homo sapiens
  Putative NFKB/NFKAPPAB.01 motif
gggaatttcc

SEQ ID NO: 451 - DNA, 13 bp, Homo sapiens
  Putative IKRS/IK1.01 motif
gccggggaatttc

SEQ ID NO: 452 - DNA, 22 bp, Homo sapiens
  Putative HEN1/HEN1.01 motif
ctggctgtcagctgagccgcgc

SEQ ID NO: 453 - DNA, 10 bp, Homo sapiens
  Putative AP4R/AP4.01 motif
ctcagctgac

SEQ ID NO: 454 - DNA, 13 bp, Homo sapiens
  Putative SP1F/SP1.01 motif
gctgggcggggtc

SEQ ID NO: 455 - DNA, 12 bp, Homo sapiens
  Putative EGRF/NGFIC.01 motif
tggcggaggggg

SEQ ID NO: 456 - DNA, 12 bp, Homo sapiens
  Putative EGRF/NGFIC.01 motif
cggcggtggcgg

SEQ ID NO: 457 - DNA, 12 bp, Homo sapiens
  Putative EGRF/NGFIC.01 motif
gggcggcggcgg

SEQ ID NO: 458 - DNA, 13 bp, Homo sapiens
  Putative SPIF/SP1.01 motif
ggcgggcggcggc

SEQ ID NO: 459 - DNA, 12 bp, Homo sapiens
  Putative AP2F/AP2.01 motif
cgcccgccggca

SEQ ID NO: 460 - DNA, 281 bp, Homo sapiens
  repeat element
cagctctattgaggtataatccacatgccataaaattcaccccatttgtaaatgtatgattcatggctttcaattacacttaaaaagttgtaaaaccatcattacaattcaaatttagtatatttccatcatcccccaaaaatcccctcgagttcctttgcagttcaaagccacccccaatttcaggcaactactggtctgatttctgtctttttctactttccttttctggacatttaatgtatatggagtcatagcatatgtagtctttggcatctggg

SEQ ID NO: 461 - DNA, 20 bp, Homo sapiens
  Short repeat element
ttcttttcttttccttcctt

SEQ ID NO: 462 - DNA, 328 bp, Homo sapiens
  ALU repeat element
tccttcttacctttcttccttctctctctctctctctttctttttggacagagtctcactccatggcccaggctggagtgcagtggcaccatcttggctcagcgcaacctttgactcccaggctcaagcaattctcctgcctcagcctctcaagtagctgagattacaggcacgcaccactactgcctggctaatttttatatttttagtagagatagggtttcaccatgttagccaggctggtcttgaactcctgacctcaaacgatcctcccaaagtgctgggattacaggcgtgagccaccgccctgggcctctttactttcttt

SEQ ID NO: 463 - DNA, 300 bp, Homo sapiens
  ALU repeat element
ctgggcaccgtggctcacacatgtaatcccagcactttggaaggccgaggcaggtggatcacccgaggtcaggagttcaataccaggctggtcaacatggcgaaacctcatcaatacgaaaaatgcaaaaattagcttggtgtggtggcacacgcctgtaatcccagccacttgggaggctgaggcaggagaatcactcaaacccaggaggtggagattgcagtgagctgagattatgccactgcactccagcctgggcaacagagtgagactccacctcaaaaaataaaataaaacctt